(19) United States
(12) Patent Application Publication
(10) Pub. No.: US 2026/0159839 A1
(43) Pub. Date: Jun. 11, 2026

(54) PHARMACEUTICAL COMPOSITION FOR TREATING SPINOCEREBELLAR ATAXIA

(71) Applicant: NATIONAL UNIVERSITY CORPORATION TOKYO MEDICAL AND DENTAL UNIVERSITY, Tokyo (JP)

(72) Inventors: Takanori YOKOTA, Tokyo (JP); Kinya ISHIKAWA, Tokyo (JP); Tetsuya NAGATA, Tokyo (JP); Miwa HIGASHI, Tokyo (JP); Kotaro YOSHIOKA, Tokyo (JP); Makoto KOIZUMI, Tokyo (JP); Yoshiyuki ONISHI, Tokyo (JP); Takao SHOJI, Tokyo (JP)

(73) Assignee: NATIONAL UNIVERSITY CORPORATION TOKYO MEDICAL AND DENTAL UNIVERSITY, Tokyo (JP)

(21) Appl. No.: 18/706,530

(22) PCT Filed: Nov. 7, 2022
(86) PCT No.: PCT/JP2022/041282

§ 371 Date: May 1, 2024

(30) Foreign Application Priority Data
Nov. 8, 2021 (JP) 2021-181571

(51) Int. Cl. C12N 15/113

(52) U.S. Cl. CPC C12N 15/1137; C12N 2310/11; C12N 2310/321; C12N 2310/341; C12N 2310/3515; C12Y 207/01021

(57) ABSTRACT

A nucleic acid molecule is provided having an antisense effect on the transcriptional product of a mutant gene having an abnormal repeat region. The abnormal repeat region has a 5′ region, a core repeat region, and a 3′ region. The core repeat region has a base sequence having multiple repeats of the base sequence TGGAA or a base sequence complementary thereto. The nucleic acid molecule includes a base sequence complementary to four or more consecutive bases in the abnormal repeat region in the transcriptional product and has a target binding region that is capable of hybridizing to the transcriptional product.

### TECHNICAL FIELD

The present invention relates to: a nucleic acid molecule having an antisense effect on the transcriptional product of a mutant gene comprising an abnormal repeat region; and a pharmaceutical composition for treating spinocerebellar ataxia.

### BACKGROUND ART

Spinocerebellar ataxia type 31 (SCA31) is a dominantly inherited spinocerebellar degeneration. It is known that SCA31 involves the impairment of superior vermis of the cerebellum, and in particular, degeneration and loss of cerebellar Purkinje cells.

The age of the onset of SCA31 is 45 to 77 years of age (mean age at the onset is 60 years). The onset of SCA31 occurs with swaying, and a nonambulatory condition develops over time, and though there are variations among individuals, a cane or wheelchair becomes necessary around 10 years after the onset, and thus, SCA31 is a disease which is slowly exacerbated. SCA31 is one of the most common spinocerebellar degenerations in Japanese people. The estimated number of patients with SCA31 is 2000 to 3000 in Japan.

In 2000, SCA31 was first found in a family in which SCA31 was linked to the long arm of chromosome 16 (Non Patent Literature 1). Further, in 2009, the onset of SCA31 was reported to be caused by an abnormal repeat region comprising a 5-base repeat elongated to have a length of around 2.5 to 3.8 kb in an intron shared by two genes BEAN1 (Brain Expressed Associated with NEDD4 1) and TK2 (Thymidine Kinase 2), which exist in the opposite directions to each other in the same region (Non Patent Literature 2). The 5-base repeat is expressed as the UGGAA repeat sequence in the transcriptional product of the BEAN1 gene or as the UUCCA repeat sequence in the transcriptional product of the TK2 gene. In patients with SCA31, RNA molecules containing these transcribed repeat sequences aggregate in the nucleus. The onset of spinocerebellar ataxia is considered to be caused by these RNA aggregates (RNA foci).

Examples of spinocerebellar ataxia having similar molecular pathology include spinocerebellar ataxia type 10 (SCA10). Four kinds of alleles are known as mutant forms of Ataxin 10 (ataxin-10; ATXN10) gene in SCA10. In allele B among them, abnormal repeat regions comprising AUUCC repeat sequences are transcribed to form RNA aggregates (RNA foci) (Non Patent Literature 3). The AUUCC repeat sequences in SCA10 become identical to the UUCCA repeat sequences in the TK2 transcriptional product in SCA31 by shifting the repeat units by one base.

However, effective methods for treating the SCA31 and SCA10 described above have been unknown.

### CITATION LIST

### Non Patent Literature

Non Patent Literature 1: Nagaoka U., Takashima M., Ishikawa K., et al., Neurology, 2000, 54:1971-1975.
Non Patent Literature 2: Sato N., Amino T., Kobayashi K., et al., Am J Hum Genet, 2009, 85:544-557.
Non Patent Literature 3: McFarland K. N., et al., PLoS One, 2015, 10(8): e0135906.

### SUMMARY OF INVENTION

### Technical Problem

An object of the present invention is to provide a nucleic acid molecule having an antisense effect on the transcriptional product of a mutant gene comprising an abnormal repeat region.

### Solution to Problem

The present inventors conducted diligent studies in order to solve the problems described above, and searched for a nucleic acid molecule having an antisense effect on the transcriptional product of a mutant gene comprising an SCA31 abnormal repeat region. As a result, a group of nucleic acid molecules that can effectively suppress the expression of the transcriptional product was found. The present invention is based on the above-described findings and provides the following.

(1) A nucleic acid molecule having an antisense effect on the transcriptional product of a mutant gene comprising an abnormal repeat region, wherein said abnormal repeat region consists of a 5′ region, a core repeat region, and a 3′ region, said core repeat region consists of a base sequence consisting of multiple repeats of the base sequence (5′-TGGAA-3′) of SEQ ID NO: 1 or a base sequence complementary thereto, and said nucleic acid molecule comprises a base sequence complementary to four or more consecutive bases in said abnormal repeat region in said transcriptional product, and comprises a target binding region that is capable of hybridizing to said transcriptional product.

(2) The nucleic acid molecule of (1), wherein said mutant gene is a mutant form of the Brain Expressed Associated With NEDD4 1 (BEAN1) gene, said core repeat region consists of a base sequence consisting of multiple repeats of the base sequence (5′-TGGAA-3′) of SEQ ID NO: 1, and said 5′ region comprises the base sequence (5′-TCAC-3′) of SEQ ID NO: 2.

(3) The nucleic acid molecule of (2), wherein said 5′ region consists of any base sequence selected from the group consisting of the following (a1) to (a4):
(a1) the base sequence of SEQ ID NO: 3;
(a2) the base sequence of SEQ ID NO: 4;
(a3) the base sequence (5′-TCAC-3′) of SEQ ID NO: 2; and
(a4) a base sequence formed by linking the base sequence (5′-TCAC-3′) of SEQ ID NO: 2 and a base sequence consisting of multiple repeats of the base sequence (5′-TAGAA-3′) of SEQ ID NO: 5 from 5′ side in this order.

(4) The nucleic acid molecule of any of (1) to (3), wherein said mutant gene is a mutant form of the Brain Expressed Associated With NEDD4 1 (BEAN1) gene, said core repeat region consists of a base sequence consisting of multiple repeats of the base sequence (5′-TGGAA-3′) of SEQ ID NO: 1, and said 3′ region comprises a sequence consisting of multiple repeats of the base sequence (5′-TAGAA-3′) of SEQ ID NO: 5 and a sequence consisting of multiple repeats of the base sequence of SEQ ID NO: 6.

(5) The nucleic acid molecule of (4), wherein said 3′ region consists of any base sequence selected from the group consisting of the following (b1) to (b5):
(b1) a base sequence formed by linking the base sequence of SEQ ID NO: 7, a base sequence consisting of multiple repeats of the base sequence (5′-TAGAA-3′) of SEQ ID NO: 5, and a base sequence consisting of multiple repeats of the base sequence of SEQ ID NO: 6, from 5′ side in this order;
(b2) a base sequence formed by linking the base sequence of SEQ ID NO: 9, a base sequence consisting of multiple repeats of the base sequence (5′-TAGAA-3′) of SEQ ID NO: 5, and a base sequence consisting of multiple repeats of the base sequence of SEQ ID NO: 6, from 5′ side in this order;
(b3) a base sequence formed by linking the base sequence of SEQ ID NO: 11, a base sequence consisting of multiple repeats of the base sequence (5′-TAGAA-3′) of SEQ ID NO: 5, and a base sequence consisting of multiple repeats of the base sequence of SEQ ID NO: 6, from 5′ side in this order;
(b4) a base sequence formed by linking the base sequence of SEQ ID NO: 12, a base sequence consisting of multiple repeats of the base sequence (5′-TAGAA-3′) of SEQ ID NO: 5, a base sequence consisting of multiple repeats of the base sequence of SEQ ID NO: 6, and a base sequence consisting of multiple repeats of the base sequence of SEQ ID NO: 13, from 5′ side in this order; and
(b5) a base sequence formed by linking the base sequence of SEQ ID NO: 14, a base sequence consisting of multiple repeats of the base sequence (5′-TAGAA-3′) of SEQ ID NO: 5, and a base sequence consisting of multiple repeats of the base sequence of SEQ ID NO: 6, from 5′ side in this order.

(6) The nucleic acid molecule of any of (2) to (5), wherein said target binding region comprises each of the bases which are complementary to the terminal bases of the two neighboring regions in the nonmutant region neighboring to 5′ side of said 5′ region, said 5′ region, said core repeat region, said 3′ region, and the nonmutant region neighboring to the 3′ side of said 3′ region.

(7) The nucleic acid molecule of any of (2) to (5), wherein the full-length of said target binding region is comprised in the base sequence which is complementary to any of said core repeat region or said 3′ region.

(8) The nucleic acid molecule of any of (2) to (5), wherein the full-length of said target binding region is comprised in the base sequence which is complementary to said core repeat region.

(9) The nucleic acid molecule of any of (2) to (5), which consists of any one base sequence selected from the group consisting of SEQ ID NOs: 156 to 189 and 207 to 209, provided that “u” in the sequence may be “t”, and “t” may be “u”.

(10) The nucleic acid molecule of (1), wherein said mutant gene is a mutant form of the Thymidine Kinase 2 (TK2) gene, said core repeat region consists of the base sequence which is complementary to a base sequence consisting of multiple repeats of the base sequence (5′-TGGAA-3′) of SEQ ID NO: 1, and said 5′ region comprises a sequence consisting of multiple repeats of the base sequence of SEQ ID NO: 16 and a sequence consisting of multiple repeats of the base sequence (5′-TTCTA-3′) of SEQ ID NO: 17.

(11) The nucleic acid molecule of (10), wherein said 5′ region consists of any base sequence selected from the group consisting of the following (c1) to (c5):
(c1) a base sequence formed by linking a base sequence consisting of multiple repeats of the base sequence of SEQ ID NO: 16, a base sequence consisting of multiple repeats of the base sequence (5′-TTCTA-3′) of SEQ ID NO: 17, and the base sequence of SEQ ID NO: 19 from 5′ side in this order;
(c2) a base sequence formed by linking a base sequence consisting of multiple repeats of the base sequence of SEQ ID NO: 16, a base sequence consisting of multiple repeats of the base sequence (5′-TTCTA-3′) of SEQ ID NO: 17, and the base sequence of SEQ ID NO: 21 from 5′ side in this order;
(c3) a base sequence formed by linking a base sequence consisting of multiple repeats of the base sequence of SEQ ID NO: 16, a base sequence consisting of multiple repeats of the base sequence (5′-TTCTA-3′) of SEQ ID NO: 17, and the base sequence of SEQ ID NO: 22 from 5′ side in this order;
(c4) a base sequence formed by linking the base sequence of SEQ ID NO: 23, a base sequence consisting of multiple repeats of the base sequence of SEQ ID NO: 16, a base sequence consisting of multiple repeats of the base sequence (5′-TTCTA-3′) of SEQ ID NO: 17, and the base sequence of SEQ ID NO: 24 from 5′ side in this order; and
(c5) a base sequence formed by linking a base sequence consisting of multiple repeats of the base sequence of SEQ ID NO: 16, a base sequence consisting of multiple repeats of the base sequence (5′-TTCTA-3′) of SEQ ID NO: 17, and the base sequence of SEQ ID NO: 29 from 5′ side in this order.

(12) The nucleic acid molecule of any of (1), (10), and (11), wherein said mutant gene is a mutant form of the Thymidine Kinase 2 (TK2) gene, said core repeat region consists of the base sequence which is complementary to a base sequence consisting of multiple repeats of the base sequence (5′-TGGAA-3′) of SEQ ID NO: 1, and said 3′ region comprises the base sequence (5′-GTGA-3′) of SEQ ID NO: 26.

(13) The nucleic acid molecule of (12), wherein said 3′ region consists of any base sequence selected from the group consisting of the following (d1) to (d4):
(d1) the base sequence of SEQ ID NO: 27;
(d2) the base sequence of SEQ ID NO: 28;
(d3) the base sequence (5′-GTGA-3′) of SEQ ID NO: 26; and
(d4) the base sequence formed by linking a base sequence consisting of multiple repeats of the base sequence (5′-TTCTA-3′) of SEQ ID NO: 17 and the base sequence (5′-GTGA-3′) of SEQ ID NO: 26 from 5′ side in this order.

(14) The nucleic acid molecule of any of (10) to (13), wherein said target binding region comprises each of the bases which are complementary to the terminal bases of the two neighboring regions in said 5′ region, said core repeat region, said 3′ region, and the nonmutant region neighboring to the 3′ side of said 3′ region.

(15) The nucleic acid molecule of any of (10) to (13), wherein the full-length of said target binding region is comprised in the base sequence which is complementary to any of said 5′ region or said core repeat region.

(16) The nucleic acid molecule of any of (10) to (13), wherein the full-length of said target binding region is comprised in the base sequence which is complementary to said core repeat region.

(17) The nucleic acid molecule of any of (10) to (13), which consists of any one base sequence selected from the group consisting of SEQ ID NOs: 33 to 53, 58 to 139, 141 to 150, 152 to 155, and 204 to 206, provided that “u” in the sequence may be “t”, and “t” may be “u”.

(18) The nucleic acid molecule of (1), wherein said mutant gene is a mutant form of the Ataxin 10 (ATXN10) gene, said core repeat region consists of the base sequence which is complementary to a base sequence consisting of multiple repeats of the base sequence (5′-TGGAA-3′) of SEQ ID NO: 1, and the full-length of said target binding region is comprised in the base sequence complementary to said core repeat region.

(19) The nucleic acid molecule of (18), which consists of any one base sequence selected from the group consisting of SEQ ID NOs: 33 to 37, 58 to 61, 64 to 67, 70 to 139, 141 to 150, 152 to 155, and 204 to 206, provided that “u” in the sequence may be “t”, and “t” may be “u”.

(20) The nucleic acid molecule of any of (1) to (19), which is 12 to 30 bases in length.

(21) The nucleic acid molecule of any of (1) to (20), which is a mixmer.

(22) The nucleic acid molecule of any of (1) to (20), which is a gapmer.

(23) The nucleic acid molecule of (22), comprising: [1] a central region which comprises at least two consecutive deoxyribonucleosides; [2] a 5′-wing region which comprises a non-natural nucleoside, positioned on the 5′ end side of said central region; and [3] a 3′-wing region which comprises a non-natural nucleoside, positioned on the 3′ end side of said central region.

(24) The nucleic acid molecule of (23), wherein said 5′-wing region and said 3′-wing region comprise a bridged nucleoside and/or a 2′-modified nucleoside.

(25) The nucleic acid molecule of (24), wherein said bridged nucleoside is LNA nucleoside or ENA nucleoside.

(26) The nucleic acid molecule of (24) or (25), wherein the 2-modification group of said 2′-modified nucleoside is 2′-O-methyl group or 2′-O-methoxyethyl group.

(27) The nucleic acid molecule of any of (1) to (20), comprising or consisting of morpholino nucleic acids.

(28) The nucleic acid molecule of any of (1) to (27), wherein all or some of the internucleoside linkages in said nucleic acid molecule are modified internucleoside linkages.

(29) The nucleic acid molecule of (28), wherein said modified internucleoside linkages are phosphorothioate linkages.

(30) The nucleic acid molecule of any of (1) to (29), comprising a modified nucleobase.

(31) The nucleic acid molecule of any of (1) to (30), wherein said antisense effect is reduction of the level of said transcriptional product (preferably the reduction caused by the decomposition of said transcriptional product).

(32) The nucleic acid molecule of any of (1) to (30), wherein said antisense effect is steric blocking.

(33) A double-stranded nucleic acid complex comprising a first nucleic acid strand consisting of the nucleic acid molecule of any of (1) to (32) and a second nucleic acid strand comprising a base sequence complementary to said first nucleic acid strand.

(34) The double-stranded nucleic acid complex of (33), wherein said second nucleic acid strand comprises a ribonucleoside, deoxyribonucleoside, and/or modified nucleoside.

(35) The double-stranded nucleic acid complex of (33) or (34), wherein in said second nucleic acid strand, all of the nucleosides in the region consisting of the base sequence complementary to the central region in said first nucleic acid strand are: (a) deoxyribonucleosides; (b) deoxyribonucleosides and ribonucleosides; (c) deoxyribonucleosides and 2′-modified nucleosides; (d) ribonucleosides and 2′-modified nucleosides; or (e) deoxyribonucleosides, ribonucleosides, and 2′-modified nucleosides.

(36) A double-stranded nucleic acid complex comprising a first nucleic acid strand consisting of the nucleic acid molecule of any of (23) to (26) and a second nucleic acid strand comprising a base sequence complementary to said first nucleic acid strand, wherein said second nucleic acid strand comprises a region comprising at least two consecutive ribonucleosides and/or deoxyribonucleosides which are complementary to at least two consecutive deoxyribonucleosides in said central region in said first nucleic acid strand.

(37) The double-stranded nucleic acid complex of (36), wherein said second nucleic acid strand comprises a modified internucleoside linkage in the region consisting of the base sequence complementary to the 5′-wing region and/or 3′-wing region of said first nucleic acid strand.

(38) The double-stranded nucleic acid complex of (37), wherein said modified internucleoside linkage is phosphorothioate linkage.

(39) The double-stranded nucleic acid complex of any of (36) to (38), wherein said second nucleic acid strand comprises a bridged nucleoside and/or 2′-modified nucleoside in the region consisting of the base sequence complementary to the 5′-wing region and/or 3′-wing region of said first nucleic acid strand.

(40) The double-stranded nucleic acid complex of (39), wherein said bridged nucleoside in said second nucleic acid strand is LNA nucleoside, ENA nucleoside, or BNA^{NC} nucleoside.

(41) The double-stranded nucleic acid complex of (39) or (40), wherein the 2′-modification group in said 2′-modified nucleoside in said second nucleic acid strand is 2′-O-methyl group or 2′-O-methoxyethyl group.

(42) The double-stranded nucleic acid complex of any of (33) to (41), wherein said second nucleic acid strand comprises one or more 2′-O-methoxyethyl-modified nucleosides.

(43) The double-stranded nucleic acid complex of (42), wherein at least 20% of the total number of nucleosides in said second nucleic acid strand are 2′-O-methoxyethyl-modified nucleosides.

(44) The double-stranded nucleic acid complex of any of (33) to (43), wherein said second nucleic acid strand comprises one or two or more consecutive 2′-O-methoxyethyl-modified nucleosides positioned at the 5′-end and/or one or two or more consecutive 2′-O-methoxyethyl-modified nucleosides positioned at the 3′-end.

(45) The double-stranded nucleic acid complex of any of (33) to (44), wherein said second nucleic acid strand comprises one to seven 2′-O-methoxyethyl-modified nucleosides at the positions other than the 5′-end and 3′-end.

(46) The double-stranded nucleic acid complex of any of (33) to (45), wherein all of the nucleosides other than 2′-O-methoxyethyl-modified nucleosides in said second nucleic acid strand are deoxyribonucleosides.

(47) The double-stranded nucleic acid complex of any of (33) to (45), wherein all of the nucleosides in said second nucleic acid strand are 2′-O-methoxyethyl-modified nucleosides.

(48) The double-stranded nucleic acid complex of any of (33) to (47), wherein said second nucleic acid strand comprises a modified nucleobase.

(49) The double-stranded nucleic acid complex of any of (33) to (48), wherein said second nucleic acid strand is bound to tocopherol or cholesterol or an analog thereof.

(50) The double-stranded nucleic acid complex of any one of claims 33 to 49, wherein the base sequence of said first nucleic acid strand consists of the base sequence of SEQ ID NO: 65, provided that “t” in the sequence may be “u”, and the base sequence of said second nucleic acid strand consists of any base sequence selected from the group consisting of SEQ ID NO: 140, SEQ ID NO: 200, SEQ ID NO: 201, SEQ ID NO: 202, and SEQ ID NO: 203, provided that “t” in the sequence may be “u”.

(51) The double-stranded nucleic acid complex of claim 50, wherein said first nucleic acid strand consists of the nucleic acid strand of SEQ ID NO: 65, and said second nucleic acid strand consists of any nucleic acid strand selected from the group consisting of SEQ ID NO: 140, SEQ ID NO: 200, SEQ ID NO: 201, SEQ ID NO: 202, and SEQ ID NO: 203.

(52) A pharmaceutical composition for treating spinocerebellar ataxia type 31 (SCA31), comprising: the nucleic acid molecule of any of (2) to (9) and (20) to (32) which refer thereto, or the double-stranded nucleic acid complex of any of (33) to (49) which refer thereto, and/or the nucleic acid molecule of any of (10) to (19) and (20) to (32) which refer thereto, or the double-stranded nucleic acid complex of any of (33) to (51) which refer thereto.

(53) A pharmaceutical composition for treating spinocerebellar ataxia type 10 (SCA10), comprising: the nucleic acid molecule of any of (18) or (19) and (20) to (32) which refer thereto, or the double-stranded nucleic acid complex of any of (33) to (51) which refer thereto.

(54) The pharmaceutical composition of (52) or (53), which is intraventricularly administered or intrathecally administered.

(55) The pharmaceutical composition of any of (52) to (54), wherein a single dose of said nucleic acid molecule is 0.1 mg/kg or more.

(56) A method for treating spinocerebellar ataxia type 31 (SCA31), comprising administering to a patient an effective amount of the nucleic acid molecule of any of (2) to (9) and (20) to (32) which refer thereto, or the double-stranded nucleic acid complex of any of (33) to (49) which refer thereto, and/or the nucleic acid molecule of any of (10) to (19) and (20) to (32) which refer thereto, or the double-stranded nucleic acid complex of any of (33) to (51) which refer thereto.

(57) A method for treating spinocerebellar ataxia type 10 (SCA10), comprising administering to a patient an effective amount of the nucleic acid molecule of any of (18) or (19) and (20) to (32) which refer thereto, or the double-stranded nucleic acid complex of any of (33) to (51) which refer thereto.

(58) The nucleic acid molecule of any of (2) to (9) and (20) to (32) which refer thereto, or the double-stranded nucleic acid complex of any of (33) to (49) which refer thereto, and/or the nucleic acid molecule of any of (10) to (19) and (20) to (32) which refer thereto, or the double-stranded nucleic acid complex of any of (33) to (51) which refer thereto, for use in the treatment of spinocerebellar ataxia type 31 (SCA31).

(59) The nucleic acid molecule of any of (18) or (19) and (20) to (32) which refer thereto, or the double-stranded nucleic acid complex of any of (33) to (51) which refer thereto, for use in the treatment of spinocerebellar ataxia type 10 (SCA10).

(60) Use of: the nucleic acid molecule of any of (2) to (9) and (20) to (32) which refer thereto, or the double-stranded nucleic acid complex of any of (33) to (49) which refer thereto, and/or the nucleic acid molecule of any of (10) to (19) and (20) to (32) which refer thereto, or the double-stranded nucleic acid complex of any of (33) to (51) which refer thereto, in the production of a therapeutic agent for spinocerebellar ataxia type 31 (SCA31).

(61) Use of the nucleic acid molecule of any of (18) or (19) and (20) to (32) which refer thereto, or the double-stranded nucleic acid complex of any of (33) to (51) which refer thereto in the production of a therapeutic agent for spinocerebellar ataxia type 10 (SCA10).

The contents of the disclosures in Japanese Patent Application No. 2021-181571 which form the basis for priority of the present application are incorporated herein.

### Advantageous Effects of Invention

A nucleic acid molecule having an antisense effect on the transcriptional product of a mutant gene comprising an abnormal repeat region can be provided.

### BRIEF DESCRIPTION OF DRAWING

FIG. 1 shows the structures of various natural nucleotides and non-natural nucleotides.

FIG. 2 shows the structures of various bridged nucleic acids.

FIG. 3 is a schematic view showing examples of specific embodiments of a nucleic acid molecule used in the present invention.

FIG. 4 is a schematic view showing examples of specific embodiments of a nucleic acid complex used in the present invention.

FIG. 5 shows the structure of a mutant TK2 gene comprising an abnormal repeat region. FIG. 5A shows the position of the abnormal repeat region in the mutant form of the TK2 gene. A nonmutant sequence neighboring to an SCA31 abnormal repeat region comprises a sequence having polymorphism (5′-(TAAAA)ₘ-3′; m is an integer 1 or more, for example, about 8 to 20). FIG. 5B shows sequences known as 5′ regions, a core repeat region, and 3′ regions.

FIG. 6 is a schematic view showing the structures of antisense oligonucleotides (ASOs) used in Example 1 and the positions thereof in a mutant TK2 gene. In the indication of each ASO, the capital letters indicate DNAs, and the underlined capital letters indicate LNAs (underlined C indicates 5-methylcytosine LNA).

FIG. 7 shows the effect of reducing the expression of SCA31 mutant repeats (TK2) by ASO (2 nM) in Example 2. The error bars indicate standard errors.

FIG. 8 shows the results of FISH detecting the expression of SCA31 mutant repeats (TK2) in Example 2. T-r1 (2 nM) was used as ASO for SCA31 mutant repeats (TK2). Cells in which the expression of SCA31 mutant repeats (TK2) was detected are indicated by the arrows.

FIG. 9 shows the effect of reducing the expression of SCA31 mutant repeats (TK2) by ASO (0.1 nM, 0.5 nM, or 2 nM) in Example 4. The error bars indicate standard errors.

FIG. 10 shows the effect of reducing the expression of SCA31 mutant repeats (TK2) by ASO (0.1 nM, 0.5 nM, 1 nM, or 2 nM) in Example 5. The error bars indicate standard errors.

FIG. 11 shows the effect of reducing the expression of SCA31 mutant repeats (TK2) by ASO (0.1 nM) in Example 6. The error bars indicate standard errors.

FIG. 12 shows the structures of ASOs targeting the interior part of core repeat regions. In the indication of each ASO, the capital letters indicate DNAs, the underlined capital letters indicate ENAs, and the italicized lower-case letters indicate 2′-OMe RNA.

FIG. 13 shows the effect of reducing the expression of SCA31 mutant repeats (TK2) by ASO (0.1 nM) targeting the interior part of core repeat regions in Example 7. The error bars indicate standard errors.

FIG. 14 shows the structures of ASOs targeting the interior part of core repeat regions. In the indication of each ASO, the capital letters indicate DNAs, the underlined capital letters indicate LNAs, and the italicized lower-case letters indicate 2′-O-MOE RNA.

FIG. 15 shows the effect of reducing the expression of SCA31 mutant repeats (TK2) by ASO (0.5 nM or 2 nM) targeting the interior part of core repeat regions in Example 8.

FIG. 16 shows the effect of reducing the expression of SCA31 mutant repeats (TK2) by ASO (0.5 nM) in Example 9. The error bars indicate standard errors.

FIG. 17 shows the effect of reducing the expression of SCA31 mutant repeats (TK2) by ASO (0.5 nM) in Example 10. The error bars indicate standard errors.

FIG. 18 shows the effect of reducing the expression of SCA31 mutant repeats (TK2) by ASO (0.5 nM) in Example 11. The error bars indicate standard errors.

FIG. 19 shows the effect of reducing the expression of SCA31 mutant repeats (TK2) by ASO (0.5 nM) in Example 12. The error bars indicate standard errors.

FIG. 20 shows the effect of reducing the expression of SCA31 mutant repeats (TK2) by heteroduplex nucleic acid (0.5 nM) in Example 14. The error bars indicate standard errors.

FIG. 21 shows the effect of reducing the expression of SCA31 mutant repeats (TK2) by ASOs in Example 15. LNA/DNA gapmer (LDG), LNA/RNA gapmer (LRG), LNA/DNA mixmer (LDM), and LNA/RNA mixmer (LRM) were used as the ASOs. The error bars indicate standard errors.

FIG. 22 shows the structure of a mutant BEAN1 gene comprising an abnormal repeat region. FIG. 22A shows the position of the abnormal repeat region in the mutant form of the BEAN1 gene. A nonmutant sequence neighboring to an SCA31 abnormal repeat region comprises a sequence having polymorphism (5′-(TAAAA)ₘ-3′; m is an integer 1 or more, for example, about 8 to 20). FIG. 22B shows sequences known as 5′ regions, a core repeat region, and 3′ regions.

FIG. 23 is a schematic view showing the structures of ASOs used in Example 16 and the positions thereof in a mutant BEAN1 gene. In the indication of each ASO, the capital letters indicate DNAs, and the underlined capital letters indicate LNAs (underlined C indicates 5-methylcytosine LNA).

FIG. 24 shows the effect of reducing the expression of SCA31 mutant repeats (BEAN1) by ASO (2 nM) in Example 16. The error bars indicate standard errors.

FIG. 25 shows the effect of reducing the expression of SCA31 mutant repeats (BEAN1) by ASO (0.1 nM) in Example 18. The error bars indicate standard errors.

FIG. 26 shows the structures of ASOs targeting the interior part of core repeat regions. In the indication of each ASO, the capital letters indicate DNAs, and the italicized lower-case letters indicate 2′-O-MOE RNA.

FIG. 27 shows the effect of reducing the expression of SCA31 mutant repeats (BEAN1) by ASO (0.1 nM) targeting the interior part of core repeat regions in Example 19.

FIG. 28 shows the effect of reducing the expression of SCA31 mutant repeats (BEAN1) by ASOs in Example 20. LNA/DNA gapmer (LDG), LNA/RNA gapmer (LRG), LNA/DNA mixmer (LDM), and LNA/RNA mixmer (LRM) were used as the ASOs. The error bars indicate standard errors.

FIG. 29 shows the structures of transgene constructs used in the production of TK2-Tg and BEAN1-BAC-Tg mice. “SCA31-1” and “EX2-3” indicate positions detected by qRT-PCR.

FIG. 30 shows the position of injection into the cerebral ventricle of a mouse in Example 21. “+” indicates the bregma, and “o” indicates the injection position.

FIG. 31 shows the effect of reducing the expression of SCA31 mutant repeats (TK2) by ASO or HDO in TK2-Tg mice in Example 21. FIG. 31A shows the results of qRT-PCR showing the effect of reducing the expression of the SCA31 mutant repeats (TK2) by T-re10a ASO. Tests were conducted in the TK2-Tg mice, and the mean value thereof is shown. FIG. 31B shows the results of qRT-PCR showing the effect of reducing the expression of the SCA31 mutant repeats (TK2) by T-re10a-HDO. The error bars indicate standard errors. FIG. 31C shows the results of Northern blot showing the effect of reducing the expression of the SCA31 mutant repeats (TK2) by T-re10a-HDO. The positions of bands corresponding to the SCA31 mutant repeats (TK2) are indicated by the arrow.

FIG. 32 shows the effect of reducing the expression of SCA31 mutant repeats by ASO in Example 21. FIG. 32A shows the results of qRT-PCR showing the effect of the expression of reducing SCA31 mutant repeats (TK2) by T-r2′_1 ASO in TK2-Tg mice.

FIG. 32B shows the results of qRT-PCR showing the effect of reducing the expression of SCA31 mutant repeats (BEAN1) by B-r2 ASO in BEAN-BAC-Tg mice. The error bars indicate standard errors.

FIG. 33 is a schematic view showing the structures of HDOs used in Example 22. FIG. 33A shows the structure of T-re10a HDO (Default). FIG. 33B shows the structure of T-re10a HDO (cMOE/DNA). FIG. 33C shows the structure of T-re10a HDO (Full cMOE). FIG. 33D shows the structure of T-re10a HDO (cMOE/DNA 2). FIG. 33E shows the structure of T-re10a HDO (cMOE/DNA_3).

FIG. 34 shows the effect of reducing the expression of SCA31 mutant repeats (TK2) by HDO in Example 22. The error bars indicate standard errors.

FIG. 35 shows the effect of reducing the expression of SCA31 mutant repeats (TK2) by HDO in Example 22. The error bars indicate standard errors.

FIG. 36 shows the structures of pkSCX-IRES-EGFP vectors comprising SCA31 insertion sequences in the TK2 direction or the BEAN1 direction. FIG. 36A shows the pkSCX-IRES-EGFP vector comprising the SCA31 insertion sequence in the TK2 direction.

FIG. 36B shows the pkSCX-IRES-EGFP vector comprising the SCA31 insertion sequence in the BEAN1 direction. “SCA31-0” indicates a position detected by qRT-PCR.

FIG. 37 shows the effect of reducing the expression of SCA31 mutant repeats (TK2) by HDO in Example 22. The diagram shows the mean values (n=3 to 9) of the results in the left cerebellum, and the error bars indicate standard errors.

### DESCRIPTION OF EMBODIMENTS

### <Nucleic Acid Molecule>

In one aspect, the present invention relates to a nucleic acid molecule. The nucleic acid molecule of the present invention has an antisense effect on the transcriptional product of a mutant gene comprising an abnormal repeat region. The nucleic acid molecule of the present invention comprises a base sequence complementary to four or more consecutive bases in the abnormal repeat region, and comprises a target binding region that is capable of hybridizing to the transcriptional product of the mutant gene comprising the abnormal repeat region.

An “abnormal repeat region” herein is a region consisting of an abnormal base sequence found in the mutant gene (a mutant BEAN1 gene, a mutant TK2 gene, or a mutant ATXN10 gene) of a patient with spinocerebellar ataxia type 31 (SCA31) or type 10 (SCA10). The abnormal repeat region is absent in the genes of a normal individual, means the whole region of an abnormal sequence present only in SCA31 or SCA10, and is used herein as a term that can encompass both the parts of a repeat sequence and a nonrepetitive sequence. The abnormal repeat region is any of a region consisting of an abnormal base sequence comprised in the intron of a mutant BEAN1 gene in a patient with spinocerebellar ataxia type 31 (SCA31), a region consisting of an abnormal base sequence comprised in the intron of a mutant TK2 gene in a patient with spinocerebellar ataxia type 31 (SCA31), or a region consisting of an abnormal base sequence comprised in the intron of a mutant ATXN10 gene in a patient having the allele B of spinocerebellar ataxia type 10 (SCA10). In particular, the abnormal repeat regions in SCA31 and SCA10 are referred to herein as “SCA31 abnormal repeat region” and “SCA10 abnormal repeat region”, respectively. The specific position of the SCA31 abnormal repeat region can be specified, for example, between the positions 68486 and 68487 in NCBI Reference Sequence: NG_021403.2.

A “target gene” herein refers to a gene to which the first nucleic acid strand of the nucleic acid molecule or double-stranded nucleic acid complex of the present invention can bind. Specifically, such a target gene corresponds to any of a mutant BEAN1 gene comprising an abnormal repeat region, a mutant TK2 gene comprising an abnormal repeat region, or a mutant ATXN10 gene comprising an abnormal repeat region.

A “target transcriptional product” means herein any RNA that is synthesized by an RNA polymerase and is a direct target of the nucleic acid molecule or nucleic acid complex of the present invention. Specifically, such a target transcriptional product is RNA that is transcribed from the target mutant gene of the present invention and comprises an RNA sequence corresponding to an abnormal repeat region. Such a target transcriptional product may be any of mature mRNA, an mRNA precursor, an RNA fragment derived from an mRNA precursor (for example, an intron moiety spliced out of an mRNA precursor), and the like. However, such a target transcriptional product is preferably an mRNA precursor. Examples of the target transcriptional product include RNA that is the transcriptional product of a mutant BEAN1 gene, RNA that is the transcriptional product of a mutant TK2 gene, and RNA that is the transcriptional product of a mutant ATXN10 gene.

An “antisense oligonucleotide (ASO)” or “antisense nucleic acid” herein means a single-stranded oligonucleotide that comprises a complementary base sequence capable of hybridizing to all or part, such as any target region, of a target transcriptional product, and can regulate the expression of a transcriptional product of the target gene or the level of the target transcriptional product by an antisense effect.

An “antisense effect” herein means an effect of regulating expression or editing of a target transcriptional product by hybridization of ASO to the target transcriptional product. The phrase “regulating expression or editing of a target transcriptional product” includes suppression or reduction of the expression of a target gene or the expression amount of a target transcriptional product (“expression amount of a target transcriptional product” is herein often referred to as “expression level of a target transcriptional product”), inhibition of translation, RNA editing, splicing function modification effect (e.g., splicing switching, exon inclusion, and exon skipping), or degradation of a transcriptional product. For example, in the case of post-transcriptional inhibition of a target gene, when an RNA oligonucleotide is introduced into a cell as ASO, the ASO forms a partial double strand by annealing to mRNA which is a transcriptional product of a target gene. This partial double strand serves as a cover to prevent translation by ribosomes, so as to inhibit the expression of the target protein encoded by the target gene at the translation level (steric blocking). On the other hand, when an oligonucleotide comprising DNA is introduced into a cell as ASO, a partial DNA-RNA heteroduplex is formed. This hetero double-stranded structure is recognized by RNase H, and as a result mRNA of the target gene is degraded and the expression of the protein encoded by the target gene is inhibited at the expression level. In addition, an antisense effect can also be produced for an intron in an mRNA precursor as a target. Further, an antisense effect can also be produced for non-coding RNA such as miRNA as a target (steric blocking). In this case, as a result of functional inhibition of the non-coding RNA, the expression or function of a gene targeted by the non-coding RNA may be influenced. For example, in the case of the functional inhibition of miRNA, the expression of a gene of which the expression is typically controlled by the miRNA can be increased. In one embodiment, the antisense effect is a decrease in the level of a transcriptional product and/or steric blocking. Commonly, when ASO comprising DNA is introduced into a cell, the ASO hybridizes to target gene mRNA having a sequence complementary to the ASO, and a DNA-RNA heteroduplex is formed. RNase H recognizes the structure of the heteroduplex and decomposes the target gene mRNA. Accordingly, the above-described decrease in the level of the transcriptional product is also preferably caused by the decomposition of a transcriptional product.

The term “nucleic acid” or “nucleic acid molecule” used herein means a nucleoside or a nucleotide in the case of a monomer, an oligonucleotide in the case of an oligomer, and a polynucleotide in the case of a polymer.

A “nucleoside” generally means a molecule consisting of a combination of a base and a sugar. The sugar moiety of a nucleoside is usually, but not limited to, composed of pentofuranosyl sugar, and specific examples thereof include ribose and deoxyribose. The base moiety of nucleoside (nucleobase) is usually a heterocyclic base moiety. Without limitation, examples thereof include adenine, cytosine, guanine, thymine, or uracil as well as other modified nucleobases (modified bases).

A “nucleotide” refers to a molecule in which a phosphate group is covalently bonded to the sugar moiety of the nucleoside. In the case of a nucleotide comprising pentofuranosyl sugar, a phosphate group is usually linked to a hydroxyl group at the 2′, 3′, or 5′ position of the sugar.

An “oligonucleotide” refers to a linear oligomer formed by linking several to dozens of neighboring nucleotides through a covalent bond between a hydroxyl group and a phosphate group in the sugar moiety. Further, a “polynucleotide” refers to a linear polymer formed by linking with covalent bonds dozens or more, preferably hundreds or more of nucleotides, namely more nucleotides than in an oligonucleotide. The nucleosides of an oligonucleotide or a polynucleotide are linked by a phosphodiester linkage.

A “nucleic acid strand” or simply “strand” herein means an oligonucleotide or a polynucleotide. A full-length strand or a partial length strand of a nucleic acid strand can be produced by a chemical synthesis using an automated synthesizer, or by an enzymatic step using a polymerase, a ligase, or a restricted enzyme reaction. A nucleic acid strand may comprise a natural nucleotide and/or a non-natural nucleotide.

A “natural nucleoside” refers herein to a nucleoside that exists in nature. Examples thereof include a ribonucleoside consisting of a ribose and the aforementioned base such as adenine, cytosine, guanine, or uracil, or a deoxyribonucleoside consisting of a deoxyribose and the aforementioned base such as adenine, cytosine, guanine, or thymine. In this regard, a ribonucleoside found in RNA, and a deoxyribonucleoside found in DNA are herein often referred to as “DNA nucleoside” and “RNA nucleoside”, respectively.

A “natural nucleotide” means herein a nucleotide that exists in nature, namely a molecule in which a phosphate group is covalently bound to the sugar moiety of the aforementioned natural nucleoside. Examples thereof include a ribonucleotide which is known as a constituent of RNA, and in which a phosphate group is bound to a ribonucleoside, and a deoxyribonucleotide, which is known as a constituent of DNA, and in which a phosphate group is bound to a deoxyribonucleoside.

A “non-natural nucleoside” means herein any nucleoside other than a natural nucleoside. For example, it comprises a modified nucleoside and a nucleoside mimic. A “modified nucleoside” means herein a nucleoside having a modified sugar moiety and/or a modified nucleobase. A nucleic acid strand comprising a non-natural oligonucleotide is in many cases more preferable than a natural type owing to desirable properties, such as enhanced cellular uptake, enhanced affinity for a target nucleic acid, increased stability in the presence of a nuclease, or increase in inhibitory activity.

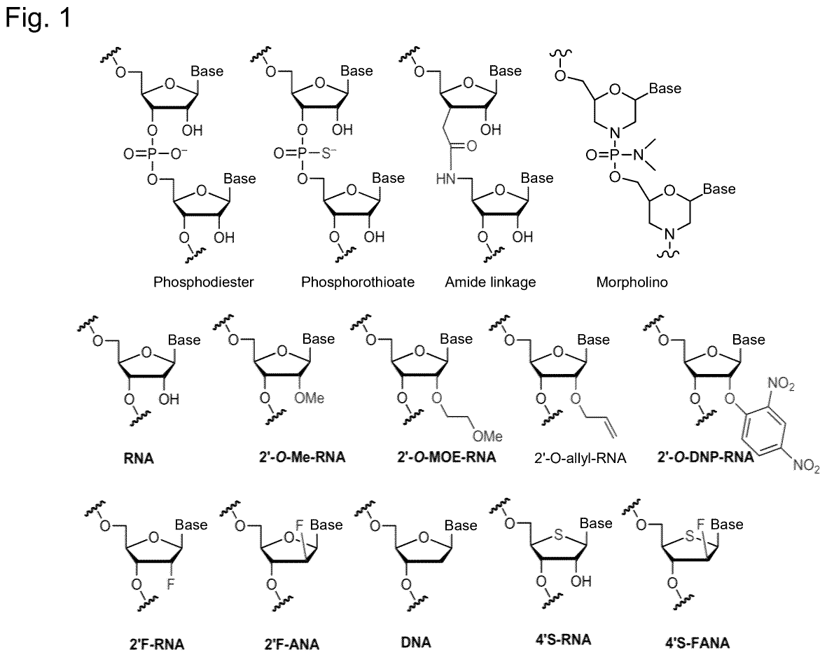

A “mimic” refers herein to a functional group that substitutes a sugar, a nucleobase, and/or an internucleoside linkage. In general, a mimic is used in place of a sugar or a combination of a sugar-internucleoside linkage, and a nucleobase is maintained for hybridization to a target to be selected. The term “nucleoside mimic” used herein comprises a structure to be used for substituting a sugar at one or more positions of an oligomer compound, or substituting a sugar and a base, or substituting a bond between monomer subunits constituting an oligomer compound. An “oligomer compound” means a polymer composed of linked monomer subunits that can at least hybridize to a region of a nucleic acid molecule. Examples of a nucleoside mimic comprise a morpholino, cyclohexenyl, cyclohexyl, tetrahydropyranyl, bicyclic or tricyclic sugar mimic, such as a nucleoside mimic having a non-furanose sugar unit. FIG. 1 shows structures of various natural or non-natural nucleotides.

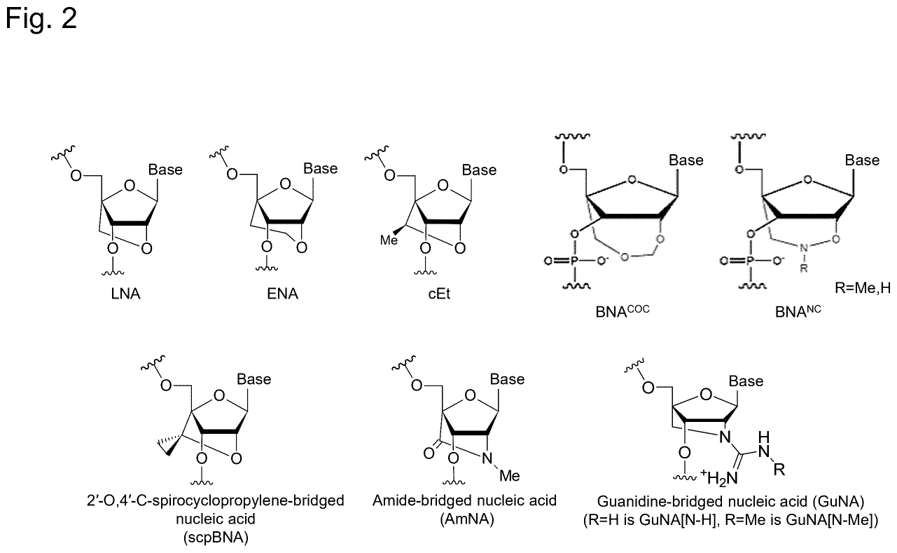

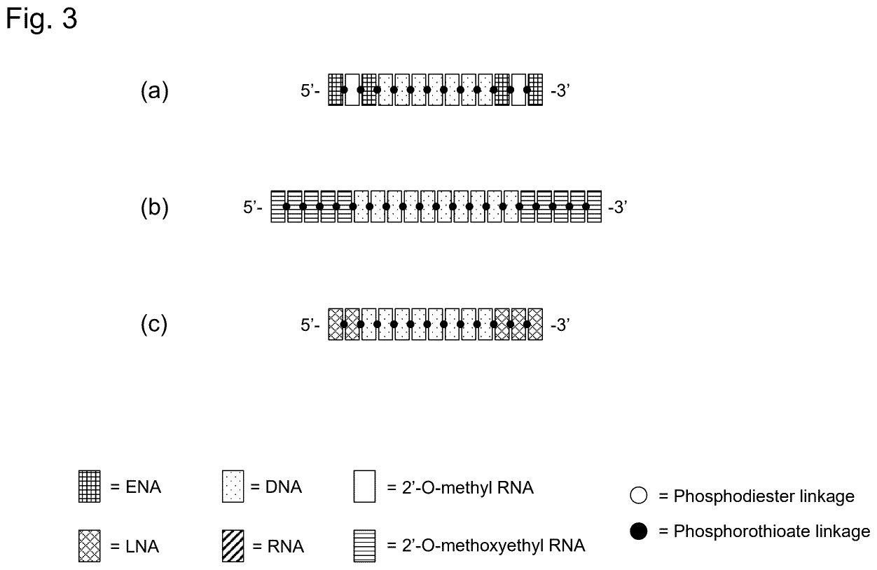

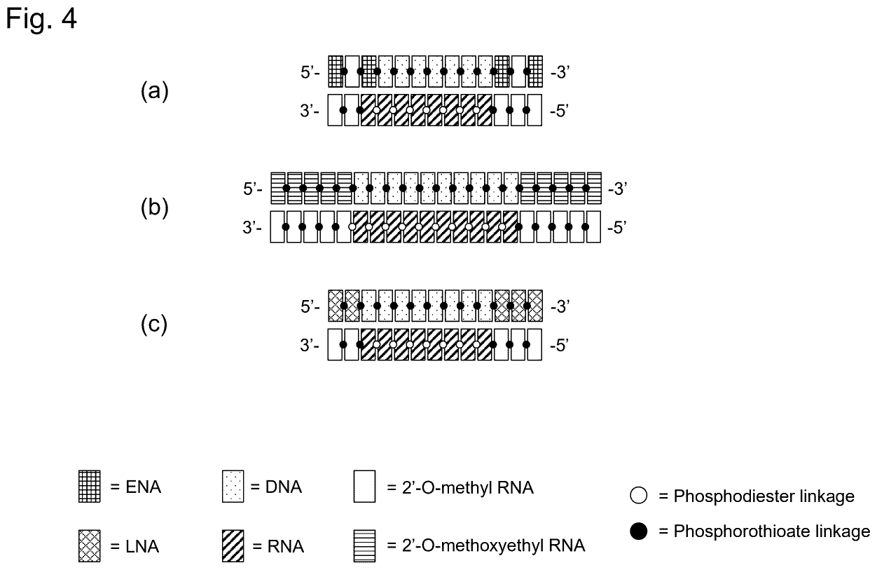

A “bicyclic nucleoside” herein means a modified nucleoside comprising a bicyclic sugar moiety. A nucleic acid comprising a bicyclic sugar moiety is commonly referred to as bridged nucleic acid (BNA). A nucleoside comprising a bicyclic sugar moiety is herein sometimes referred to as “bridged nucleoside.” Some examples of a bridged nucleic acid are shown in FIG. 2.

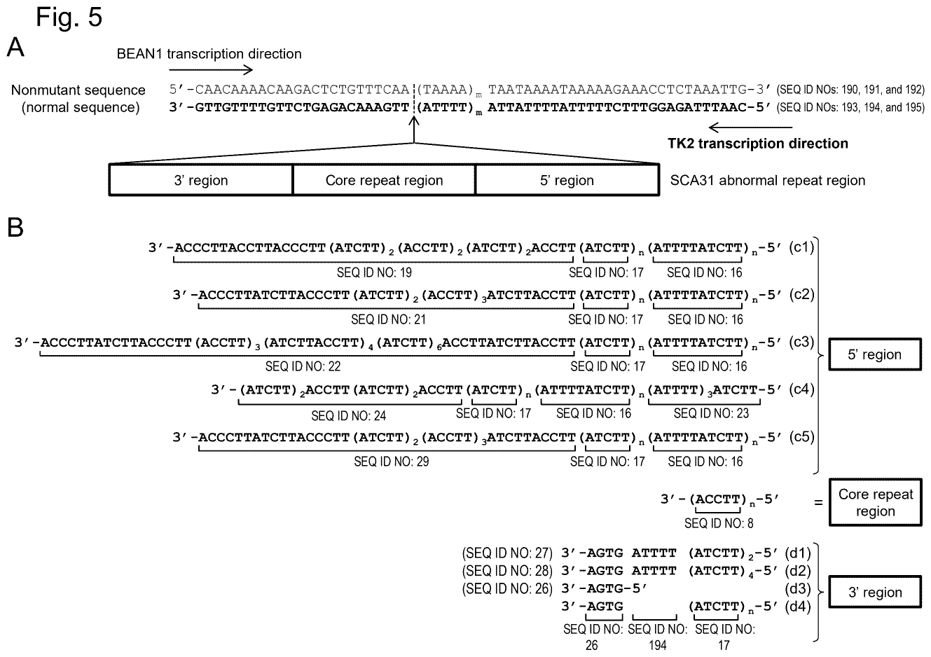

A bicyclic sugar may be a sugar in which the carbon atom at the 2′ position and the carbon atom at the 4′ position are bridged with two or more atoms. Examples of a bicyclic sugar are known to those skilled in the art. A subgroup of nucleic acids comprising a bicyclic sugar (BNA) may be so described that they have a carbon atom at the 2′ position and a carbon atom at the 4′ position which are bridged with 4′-(CH₂)ₚ—O-2′, 4′-(CH₂)ₚ—CH₂-2′, 4′-(CH₂)ₚ—S-2′, 4′-(CH₂)ₚ—OCO-2′, 4′-(CH₂)ₙ—N(R₃)—O—(CH₂)ₘ-2′ [wherein p, m, and n represent integers from 1 to 4, from 0 to 2, and from 1 to 3, respectively; and R₃ represents a hydrogen atom, an alkyl group, an alkenyl group, a cycloalkyl group, an aryl group, an aralkyl group, an acyl group, a sulfonyl group, and a unit substituent (e.g., a fluorescently or chemiluminescently labeled molecule, a functional group with nucleic acid cleavage activity, and an intracellular or intranuclear localization signal peptide)]. Furthermore, with respect to a BNA in a certain embodiment, in the OR₂ substituent of the carbon atom at the 3′ position and the OR₁ substituent of the carbon atom at the 5′ position, R₁ and R₂ are typically hydrogen atoms, but may be the same or different from each other, or may also be a protecting group for a hydroxyl group for nucleic acid synthesis, an alkyl group, an alkenyl group, a cycloalkyl group, an aryl group, an aralkyl group, an acyl group, a sulfonyl group, a silyl group, a phosphate group, a phosphate group protected by a protecting group for nucleic acid synthesis, or P(R₄)R₅ [wherein R₄ and R₅, may be the same or different from each other, and respectively represent a hydroxyl group, a hydroxyl group protected by a protecting group for nucleic acid synthesis, a mercapto group, a mercapto group protected by a protecting group for nucleic acid synthesis, an amino group, a C₁-C₅ alkoxy group, a C₁-C₅ alkylthio group, a C₁-C₆ cyanoalkoxy group, or an amino group substituted with a C₁-C₅ alkyl group]. Non-limiting examples of such BNA comprise methyleneoxy(4′-CH₂—O-2′) BNA (LNA (Locked Nucleic Acid (registered trademark), also known as 2′,4′-BNA), e.g., α-L-methyleneoxy(4′-CH₂—O-2′) BNA or β-D-methyleneoxy(4′-CH₂—O-2′) BNA, ethyleneoxy(4′-(CH₂)₂—O-2′) BNA having an ethylene bridge between the 2 position and the 4 position (“2′-O,4′-C-ethylene nucleoside”, also known as “ENA”; see Japanese Patent Laid-Open No. 2000-297097 and WO2019/009299A1), β-D-thio(4′-CH₂—S-2′) BNA, aminooxy(4′-CH₂—O—N(R₃)-2′) BNA, oxyamino(4′-CH₂—N(R)—O-2′) BNA (also known as 2′,4′-BNA^{NC}; R=H is 2′,4′-BNA^{NC}[N—H], R=Me is 2′,4′-BNA^{NC}[N-Me]), 2′,4′-BNA^{coc}, 3′-amino-2′,4′-BNA, 5′-methyl BNA, (4′-CH(CH₃)—O-2′) BNA (also known as cEt BNA), (4′-CH(CH₂OCH₃)—O-2′) BNA (also known as cMOE BNA), amido BNA (4′-C(O)—N(R)-2′) BNA (R=H, or Me) (also known as AmNA; R=H is AmNA[N—H], R=Me is AmNA[N-Me])), guanidine BNA (also known as GuNA (e.g., R=H is GuNA[N—H], R=Me is GuNA[N-Me]) in FIG. 5), amine BNA (also known as 2′-Amino-LNA), 2′-0,4′-C-spirocyclopropylene-bridged nucleic acid (also known as scpBNA), and other BNA known to those skilled in the art. A bicyclic nucleoside having a methyleneoxy(4′-CH₂—O-2′) bridge is also referred to as LNA nucleoside.

A “non-natural nucleotide” means herein any nucleotide other than a natural nucleotide. For example, it comprises a modified nucleotide and a nucleotide mimic. A “modified nucleotide” means herein a nucleotide having any one or more of a modified sugar moiety, a modified internucleoside linkage, and a modified nucleobase. The term “nucleotide mimic” herein comprises a structure used to substitute a nucleoside and a bond (linkage) at one or more positions in an oligomer compound. Examples of the nucleotide mimic comprise a peptide nucleic acid, and a morpholino nucleic acid. The peptide nucleic acid (PNA) is a nucleotide mimic having a main chain in which N-(2-aminoethyl)glycine in place of a sugar is linked with an amide linkage. The morpholino nucleic acid (MNA) is a nucleotide mimic having a main chain in which a morpholine ring is linked with a phosphorodiamidate linkage. A nucleic acid strand comprising a non-natural oligonucleotide herein has in many cases preferable properties, such as enhanced cellular uptake, enhanced affinity for a target nucleic acid, improved stability in the presence of a nuclease, and improvement in inhibitory activity. Therefore, a non-natural nucleotide is preferable to a natural nucleotide in the case of using a nucleotide as ASO.

A “modified internucleoside linkage” means herein an internucleoside linkage that has a substitution or any change from a naturally occurring internucleoside linkage (i.e., phosphodiester linkage). A modified internucleoside linkage comprises a phosphorus-containing internucleoside linkage that comprises a phosphorus atom, and a phosphorus-free internucleoside linkage that does not comprise a phosphorus atom. Typical phosphorus-containing internucleoside linkages comprise, but not limited to, a phosphorothioate linkage, a phosphorodithioate linkage, a phosphotriester linkage, an alkylphosphonate linkage, an alkylthiophosphonate linkage, and a phosphorodiamidate. A phosphorothioate linkage is an internucleoside linkage in which an unbridged oxygen atom in a phosphodiester linkage is substituted with a sulfur atom. A method for preparing a phosphorus-containing and phosphorus-free linkage is well known. It is preferable that a modified internucleoside linkage is a linkage having a higher resistance to a nuclease than a naturally occurring internucleoside linkage.

When an internucleoside linkage has a chiral center, the internucleoside linkage may be chirally controlled. The term “chirally controlled” means that a single diastereomer is present with respect to the chiral center, for example, a chiral phosphorus atom. An internucleoside linkage chirally controlled may have completely single stereochemistry, or may have a high isomeric purity, for example, an isomeric purity of 90% de, 95% de, 98% de, 99% de, 99.5% de, 99.8% de, 99.9% de, or more. An “isomeric purity” herein refers to the proportion of one diastereomer in a diastereomer mixture, is represented as the diastereomeric excess rate (% de), and is defined as (diastereomer of interest−Other diastereomers)/(Total diastereomer)×100(%).

An internucleoside linkage may be, for example, a phosphorothioate linkage chirally controlled to an Rp configuration or Sp configuration. A method for preparing an internucleoside linkage chirally controlled is publicly known, and a phosphorothioate linkage chirally controlled to an Rp configuration or Sp configuration can be synthesized according to, for example, a method described in Naoki Iwamoto et al., Angew. Chem. Int. Ed. Engl. 2009, 48(3), 496-9, Natsuhisa Oka et al., J. Am. Chem. Soc. 2003, 125, 8307-8317, Natsuhisa Oka et al., J. Am. Chem. Soc. 2008, 130, 16031-16037, Yohei Nukaga et al., J. Org. Chem. 2016, 81, 2753-2762, or Yohei Nukaga et al., J. Org. Chem. 2012, 77, 7913-7922. A phosphorothioate linkage chirally controlled to an Rp configuration or Sp configuration is also publicly known, and is known to produce an effect described in, for example, Naoki Iwamoto et al., Nat. Biotechnol., 2017, 35(9), 845-851, or Anastasia Khvorova et al., Nat. Biotechnol., 2017, 35(3), 238-248. For example, in one embodiment, a chirally controlled phosphorothioate linkage in the Sp configuration is more stable than those in the Rp configuration and/or chiral controlled ASOs in the Sp configuration promote target RNA cleavage by RNase H1, resulting in a more sustained response in vivo.

A “modified nucleobase” or a “modified base” means herein any nucleobase other than adenine, cytosine, guanine, thymine, or uracil. Examples of a modified nucleobase comprise, but not limited to, 5-methylcytosine, 5-fluorocytosine, 5-bromocytosine, 5-iodocytosine, N4-methylcytosine, N6-methyladenine, 8-bromoadenine, N2-methylguanine, or 8-bromoguanine. A preferred modified nucleobase is 5-methylcytosine. In addition, 5-methyluracil and thymine have the same structure, and may be expressed based on uracil (e.g., “5meU”) or may be expressed based on thymine (e.g., “T”).

The term “unmodified nucleobase” or “unmodified base” is synonymous with a natural nucleobase, and means adenine (A) and guanine (G), which are purine bases, and thymine (T), cytosine (C), and uracil (U), which are pyrimidine bases.

A “modified sugar” refers herein to a sugar in which a natural sugar moiety (i.e., sugar moiety found in DNA(2′-H) or RNA(2′-OH)) has undergone a substitution and/or any change. A nucleic acid strand herein may comprise in some cases one or more modified nucleosides comprising a modified sugar. A sugar-modified nucleoside can confer a beneficial biological property such as improved nuclease stability, increased binding affinity, or the like to a nucleic acid strand. A nucleoside may comprise a chemically modified ribofuranose ring moiety. Examples of a chemically modified ribofuranose ring comprise, but not limited to, addition of a substituent (comprising 5′ and 2′ substituents), formation of a bicyclic nucleic acid (bridged nucleic acid, BNA) through bridge formation in a ribofuranose ring, substitution of a ribosyl ring oxygen atom with S, N(R), or C(R1)(R2) (wherein R, R1, and R2 independently represent H, a C₁-C₁₂ alkyl, or a protecting group), and a combination thereof. Examples of a nucleoside having a modified sugar moiety herein comprise, but not limited to, a nucleoside comprising a substituent such as 5′-vinyl, 5′-methyl (R or S), 4′-S, 2′-F (2′-fluoro group), 2′-OCH₃ (2′-OMe group or 2′-O-methyl group), and 2′-O(CH₂)₂OCH₃ group (2′-O-MOE group or 2′-O-methoxyethyl group).

A “2′-modified sugar” means herein a furanosyl sugar modified at the 2′ position. Substitution of a 2′-hydroxyl group can be selected from C₁-C₁₀ alkyl, allyl, amino, azide, thio-O-allyl, —O—C₁-C₁₀ alkyl, —OCF₃, —O(CH₂)₂SCH₃, —O(CH₂)₂OCH₃, —O(CH₂)₂—O—N(Rm)(Rn), O—CH₂—C(═O)—N(Rm)(Rn), —O—(CH₂)₂—C(═O)—N(Rm)(Rn), and the like, wherein Rm and Rn are each independently H or a substituted or unsubstituted C₁-C₁₀ alkyl. Herein, a nucleoside comprising 2′-modified sugar is referred to as a “2′-modified nucleoside”. For example, a 2′-modified nucleoside in which a 2′-modified group is a 2′-O-methoxyethyl group is referred to as a 2′-O-methoxyethyl-modified nucleoside.

In general, a modification can be performed such that nucleotides in the same strand can independently undergo different modifications. In addition, to provide resistance to enzymatic cleavage, the same nucleotide can have a modified internucleoside linkage (e.g., phosphorothioate linkage) and also a modified sugar (e.g., a 2′-O-methyl modified sugar or a bicyclic sugar). The same nucleotide can also have a modified nucleobase (e.g., 5-methylcytosine) and can also have a modified sugar (e.g., a 2′-O-methyl modified sugar, or a bicyclic sugar).

The number, kind, and position of a non-natural nucleotide in a nucleic acid strand may influence the antisense effect and the like provided by the nucleic acid complex of the present invention. The choice of a modification may vary depending on the sequence of a target gene or the like, but those skilled in the art can select the modification as appropriate by referring to the descriptions in the literature related to the description herein and the antisense method (e.g., WO2007/143315, WO2008/043753, and WO2008/049085). The choice is not limited to the aspects of Examples. Furthermore, when the antisense effect of a nucleic acid complex after the modification is measured and the obtained measured value is not significantly lower than the measured value of the nucleic acid complex before the modification (e.g., when the measured value obtained after the modification is 70% or more, 80% or more, or 90% or more of the measured value of the nucleic acid complex before the modification), a relevant modification may be evaluated.

Herein, A^{t}, G^{t}, 5meC^{t}, C^{t}, T^{t}, U^{t}, A^{p}, G^{p}, 5meC^{p}, C^{p}, T^{p}, U^{p}, A^{s}, G^{s}, 5meC^{s}, C^{s}, T^{s}, U^{s}, A^{rt}, G^{rt}, C^{rt}, U^{rt}, A^{rp}, G^{rp}, C^{rs}, U^{rs}, A^{m1t}, G^{m1t}, C^{m1t}, 5meC^{m1t}, U^{m1t}, A^{m1p}, G^{m1p}, C^{m1p}, 5meC^{m1p}, U^{m1p}, A^{m1s}, G^{m1s}, C^{m1s}, 5meC^{m1s}, U^{m1s}, A^{2t}, G^{2t}, C^{2t}, T^{2t}, A^{e2p}, G^{e2p}, Ce²p, T^{e2p}, A^{e2s}, G^{e2s}, C^{e2s}, and T^{e2s} are groups having structures shown below.

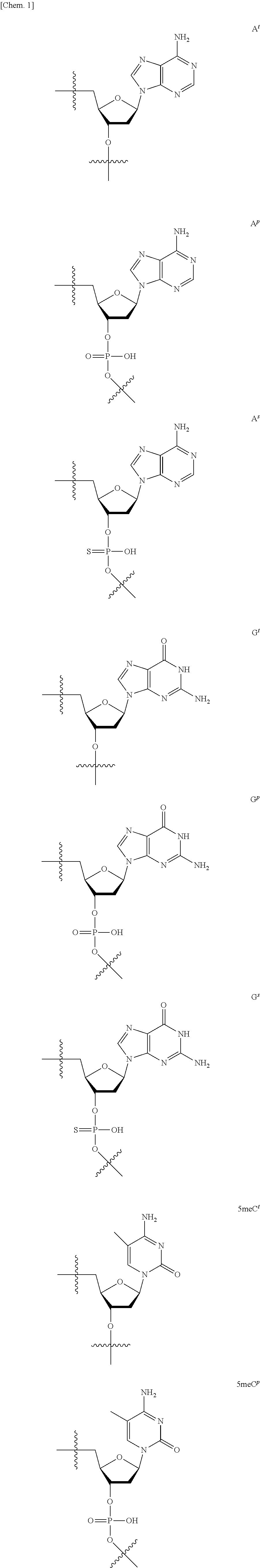

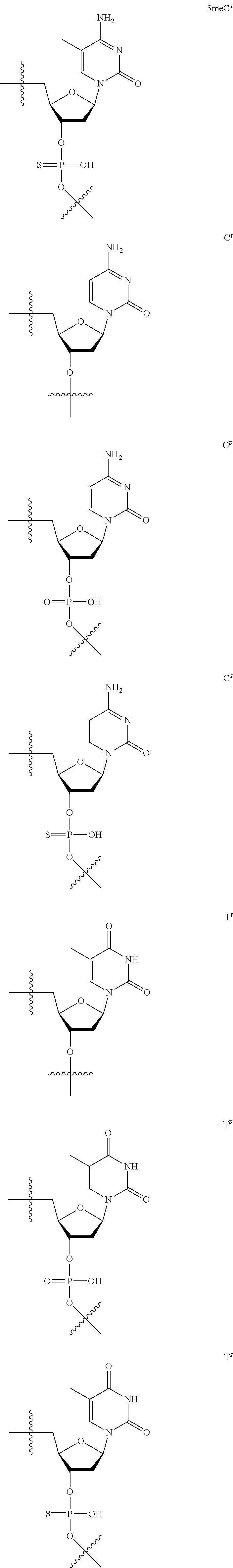

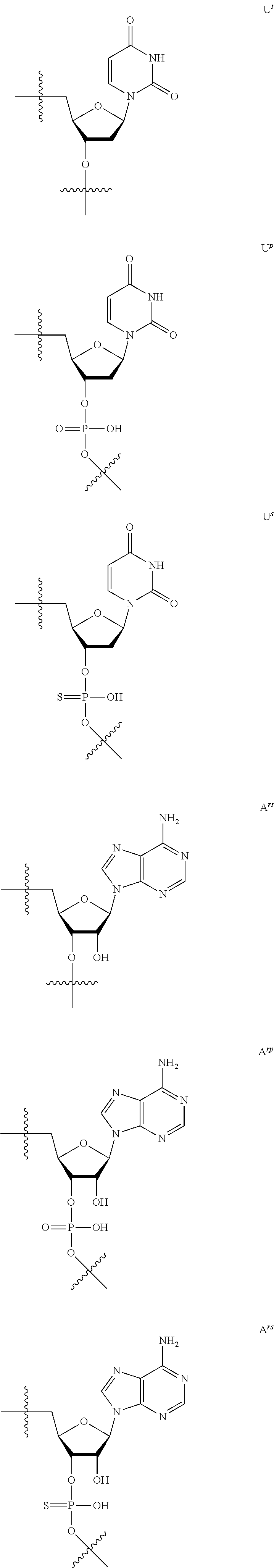

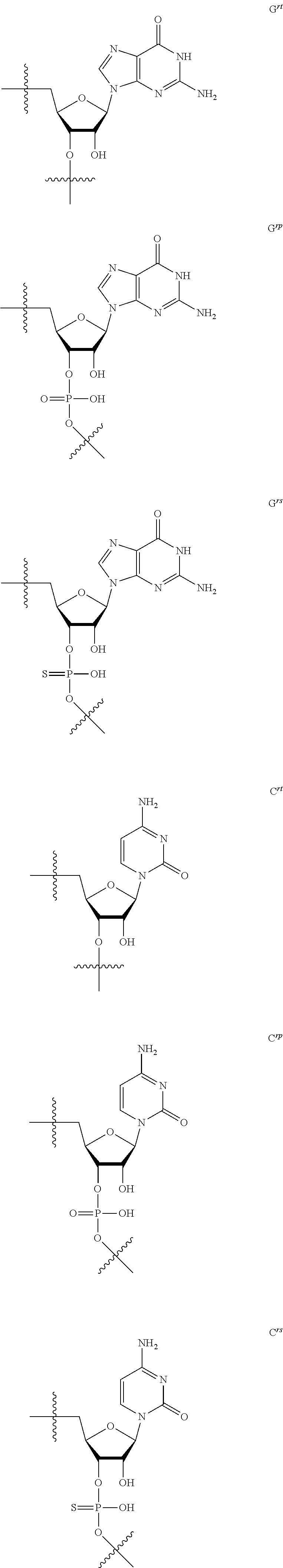

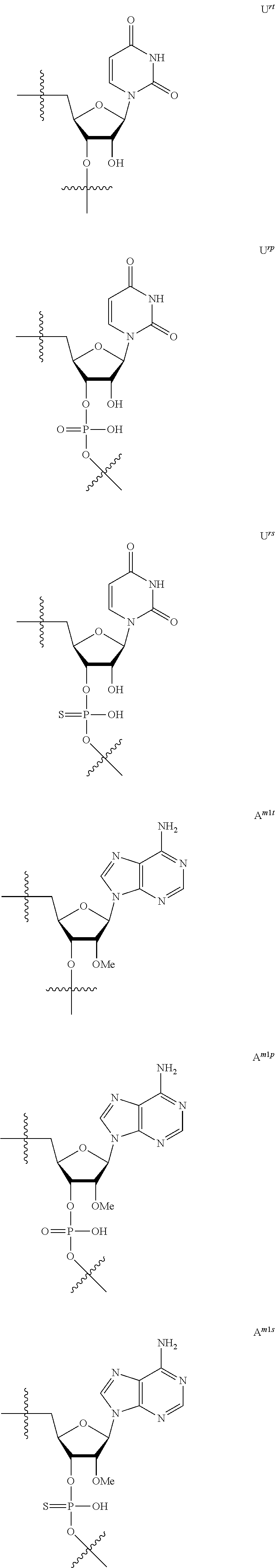

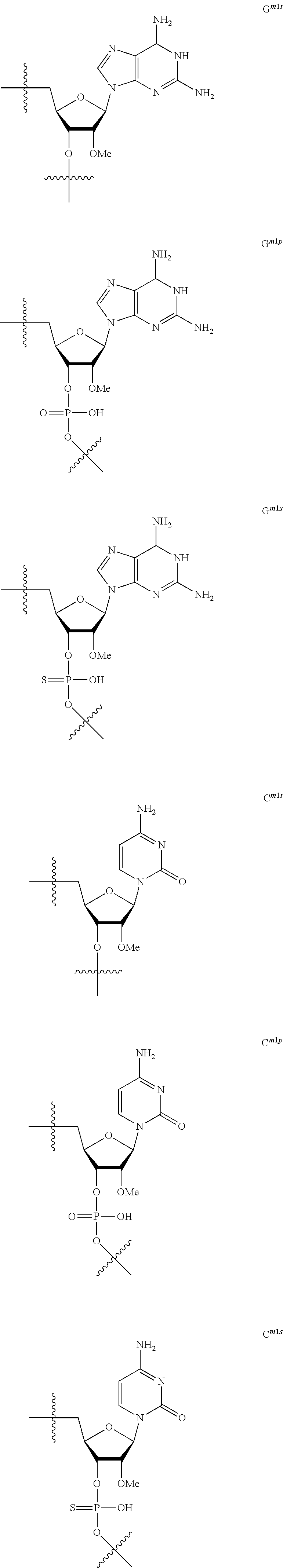

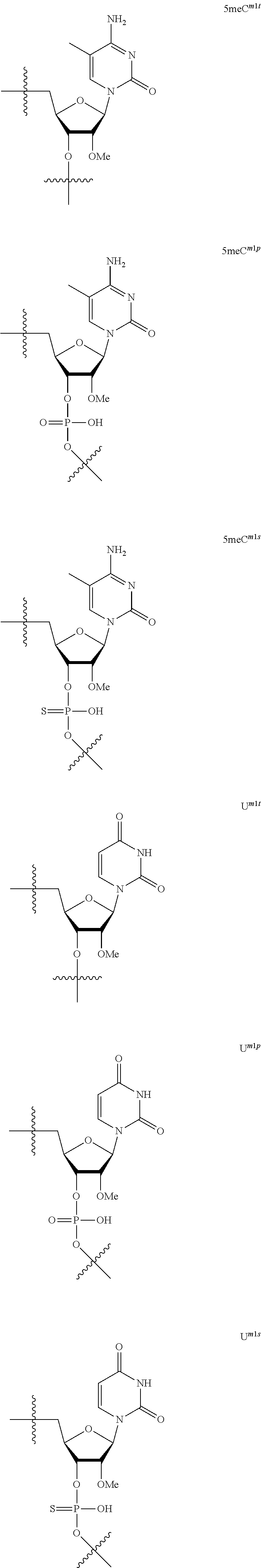

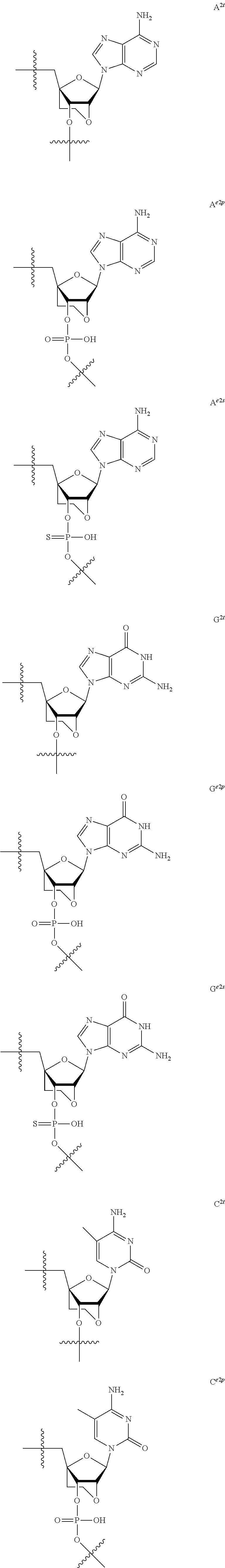

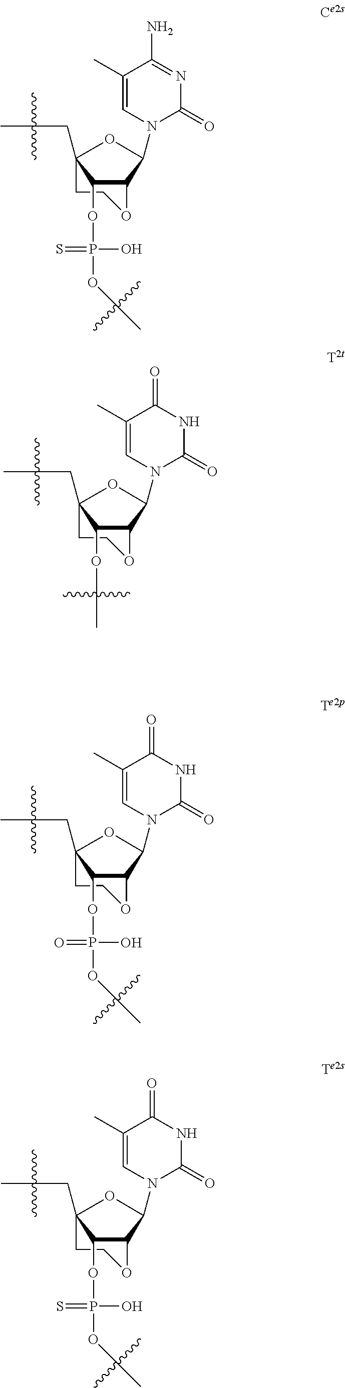 [SMILES: *C[C@@]12CCOC([C@H](n3cc(C)c(N)nc3=O)O1)[C@H]2OP(O)(=S)O* ; *C[C@@]12CCOC([C@H](n3cc(C)c(=O)[nH]c3=O)O1)[C@H]2O* ; *C[C@@]12CCOC([C@H](n3cc(C)c(=O)[nH]c3=O)O1)[C@H]2OP(=O)(O)O* ; *C[C@@]12CCOC([C@H](n3cc(C)c(=O)[nH]c3=O)O1)[C@H]2OP(O)(=S)O*]

The term “complementary” as used herein refers to the relationship that nucleobases can form via hydrogen bonds so-called Watson-Crick base pairs (natural base pairs) or Wobble base pairs (guanine-thymine or guanine-uracil). In the present invention, the nucleic acid molecule or the first nucleic acid strand is not necessarily required to be completely complementary to all or part of a target transcriptional product (e.g., the transcriptional product of a target gene), and it is acceptable if the base sequence has at least 70%, preferably at least 80%, and further preferably at least 90% (e.g., 95%, 96%, 97%, 98%, or 99% or more) in the complementarity. Similarly, the nucleic acid strand second in the double-stranded nucleic acid complex is not necessarily required to be completely complementary to all or part of the first nucleic acid strand, and it is acceptable if the base sequence has a complementarity of at least 70%, preferably at least 80%, and further preferably at least 90% (e.g., 95%, 96%, 97%, 98%, or 99% or more). Sequence complementarity can be determined by using a BLAST program or the like. The first nucleic acid strand can “hybridize” to a target transcriptional product when the sequence thereof is complementary (typically when the sequence thereof is complementary to the sequence of at least a part of the target transcriptional product). The first nucleic acid strand can “anneal” to the complementary region of the second nucleic acid strand when the sequence thereof is complementary. Those skilled in the art can easily determine the conditions (temperature, salt concentration, and the like) under which two strands can anneal or hybridize, taking into account the degree of complementarity between the strands. Such conditions may be typically physiological conditions. Furthermore, those skilled in the art can easily design an antisense nucleic acid complementary to a target transcriptional product, for example, based on information about the base sequence of a target gene.

Hybridization conditions may be variously stringent, for example, low-stringent and high-stringent conditions. Low-stringent conditions may be, e.g., 30° C., 2×SSC, 0.1% SDS. High-stringent conditions may be, e.g., 65° C., 0.1×SSC, 0.1% SDS. The stringency of hybridization can be adjusted by changing conditions such as temperature and salt concentration. Here, 1×SSC contains 150 mM sodium chloride and 15 mM sodium citrate.

“Tocopherol” is herein a methylated derivative of tocol which is a liposoluble vitamin (vitamin E) having a cyclic structure called chroman. Tocol has a strong antioxidant effect, and therefore functions in vivo as an antioxidant substance to scavenge free radicals produced by metabolism and protect cells from damage.

A plurality of different types of tocopherol are known based on the position of the methyl group bound to chroman consisting of α-tocopherol, β-tocopherol, γ-tocopherol, and δ-tocopherol. A tocopherol herein may be any types of tocopherol. In addition, examples of the analog of tocopherol comprise various unsaturated analogs of tocopherol, such as α-tocotrienol, β-tocotrienol, γ-tocotrienol, and δ-tocotrienol. Preferably, tocopherol is α-tocopherol.

“Cholesterol” is herein a kind of sterol, also called steroid alcohol, which is especially abundant in animals. Cholesterol exerts an important function in the metabolic process in vivo, and in animal cells, it is also a major constituent of the membrane system of a cell, together with phospholipid. In addition, the cholesterol analog refers to various cholesterol metabolism products and their analogs, which are alcohols having a sterol backbone. Examples thereof include, but not limited to, cholestanol, lanosterol, cerebrosterol, dehydrocholesterol, and coprostanol.

An “analog” herein refers to a compound having a similar structure and property having the same or a similar basic backbone. The analog comprises, for example, a biosynthetic intermediate, a metabolism product, and a compound having a substituent. Those skilled in the art can determine whether or not a compound is an analog of another compound based on common general technical knowledge.

A “subject” herein refers to the object to which the nucleic acid molecule, double-stranded nucleic acid complex, or pharmaceutical composition of the present invention is applied. A subject comprises an individual as well as an organ, a tissue, and a cell. When the subject is an individual, any animal including a human may be applicable. For example, in addition to a human, a variety of domestic animals, domestic fowls, pets, and laboratory animals are included. Without limitation, a subject may be an individual who needs reduction of the expression level of a target transcriptional product, preferably an individual suffering from SCA31 or SCA10, or an individual expected to suffer from SCA31 or SCA10 (e.g., an individual belonging to a family with SCA31 or SCA10 and/or an individual expected to suffer from SCA31 or SCA10 in consideration of a genotype or the like).

Unless otherwise specified, a base sequence herein is described in the order from the 5′ side to the 3′ side. Base sequences shown by the SEQ ID NOs being skipped in the sequence listing are described below. All the shown base sequences are DNA sequences derived from a human.

SEQ ID NO 1:
5′-TGGAA-3′
SEQ ID NO 2:
5′-TCAC-3′
SEQ ID NO 5:
5′-TAGAA-3′
SEQ ID NO 8:
5′-TTCCA-3′
SEQ ID NO 17:
5′-TTCTA-3′
SEQ ID NO 26:
5′-GTGA-3′
SEQ ID NO 191:
5′-TAAAA-3′
SEQ ID NO 194:
5′-TTTTA-3′
SEQ ID NO 196:
5′-TTGAAACAG-3′
SEQ ID NO 198:
5′-CTGTTTCAA-3′

The nucleic acid molecule of the present aspect is a nucleic acid molecule comprising a target binding region that is capable of hybridizing to the transcriptional product of a mutant gene comprising an abnormal repeat region, and having an antisense effect on the transcriptional product. The target binding region comprises a base sequence complementary to four or more consecutive bases in said abnormal repeat region in the transcriptional product.

The abnormal repeat region comprised by the mutant gene that encodes the transcriptional product that is targeted by the nucleic acid molecule of the present aspect consists of a 5′ region, a core repeat region, and a 3′ region.

Herein, a “core repeat region” is any of a base sequence (5′-(TGGAA)ₙ-3′; n is an integer 2 or more) consisting of multiple repeats of the base sequence (5′-TGGAA-3′) of SEQ ID NO: 1 or a base sequence complementary thereto. Here, a “base sequence complementary thereto” is a base sequence (5′-(TTCCA)ₙ-3′; n is an integer 2 or more) consisting of multiple repeats of the base sequence (5′-TTCCA-3′) of SEQ ID NO: 8. Here, “n” varies depending on a case, and is not limited to the following range. Examples of “n” include an integer from 2 to 500, from 100 to 400, or from 200 to 400.

Herein, a “5′ region” is a region positioned on the 5′ side of a core repeat region in an abnormal repeat region. A “3′ region” is a region positioned on the 3′ side of a core repeat region in an abnormal repeat region. The base sequences of the 5′ region and the 3′ region are not limited as long as being sequences that can be found in a patient with spinocerebellar ataxia of SCA31 or SCA0.

The further configurations of the nucleic acid molecule of the present invention differ according to the kinds of mutant genes as targets, and are therefore separately described below on the basis of the kinds of mutant genes.

In one embodiment, the mutant gene that encodes a transcriptional product targeted by the nucleic acid molecule of the present invention is a mutant BEAN1 gene.

In an abnormal repeat region which can be comprised by a mutant BEAN1 gene, a core repeat region is a base sequence (5′-(TGGAA)ₙ-3′; n is an integer 2 or more consisting of multiple repeats of the base sequence (5′-TGGAA-3′) of SEQ ID NO: 1. Here, “n” varies depending on a case, and is not limited to the following range. Examples of “n” include an integer from 2 to 500, from 100 to 400, or from 200 to 400.

The base sequences of the 5′ region and the 3′ region in the abnormal repeat region of the mutant form of the BEAN1 gene are not particularly limited as long as being sequences that can be found in a patient with spinocerebellar ataxia of SCA31.

Examples of the 5′ region in the abnormal repeat region of the mutant form of the BEAN1 gene include regions comprising the base sequence (5′-TCAC-3′) of SEQ ID NO: 2. Specific examples of the base sequences thereof include the following (a1) to (a4):
(a1) a base sequence (5′-TCACTAAAA(TAGAA)₂-3′) of SEQ ID NO: 3;
(a2) a base sequence (5′-TCACTAAAA(TAGAA)₄-3′) of SEQ ID NO: 4;
(a3) a base sequence (5′-TCAC-3′) of SEQ ID NO: 2; and
(a4) a base sequence formed by linking a base sequence (5′-(TAGAA)ₙ-3′; n is an integer 2 or more) consisting of multiple repeats of a base sequence (5′-TCAC-3′) of SEQ ID NO: 2 and a base sequence (5′-TAGAA-3′) of SEQ ID NO: 5 from 5′ side in this order. Here, “n” varies depending on a case, and is not limited to the following range. Examples of “n” include an integer from 2 to 500, from 100 to 400, or from 200 to 400.

Examples of the 3′ region in the abnormal repeat region of the mutant form of the BEAN1 gene include a region comprising a sequence (5′-(TAGAA)ₙ-3′; n is an integer 2 or more) consisting of multiple repeats of the base sequence (5′-TAGAA-3′) of SEQ ID NO: 5 and a sequence (5′-(TAAAATAGAA)ₙ-3′, n is an integer 2 or more) consisting of multiple repeats of the base sequence of SEQ ID NO: 6. Specific examples of the base sequences thereof include the following (b1) to (b5):
(b1) a base sequence formed by linking the base sequence of SEQ ID NO: 7 (5′-TGGGAATGGAATGGGAA(TAGAA)₂(TGGAA)₂(TAGAA)₂TGGAA-3′), a base sequence (5′-(TAGAA)ₙ-3′, n is an integer 2 or more) consisting of multiple repeats of the base sequence of SEQ ID NO: 5 (5′-TAGAA-3′), and a base sequence (5′-(TAAAATAGAA)ₙ-3′, n is an integer 2 or more) consisting of multiple repeats of the base sequence of SEQ ID NO: 6 from 5′ side in this order;
(b2) a base sequence formed by linking the base sequence of SEQ ID NO: 9 (5′-TGGGAATAGAATGGGAA(TAGAA)₂(TGGAA)₃TAGAATGGAA-3′, n is an integer 2 or more), a base sequence (5′-(TAGAA)ₙ-3′, n is an integer 2 or more) consisting of multiple repeats of the base sequence of SEQ ID NO: 5 (5′-TAGAA-3′), and a base sequence (5′-(TAAAATAGAA)ₙ-3′, n is an integer 2 or more) consisting of multiple repeats of the base sequence of SEQ ID NO: 6 from 5′ side in this order;
(b3) a base sequence formed by linking the base sequence of SEQ ID NO: 11 (5′-TGGGAATAGAATGGGAA(TGGAA)₃(TAGAATGGAA)₄(TAGAA)₆TGGAATAGAATGGAA-3′), a base sequence (5′-(TAGAA)ₙ-3′, n is an integer 2 or more) consisting of multiple repeats of the base sequence of SEQ ID NO: 5 (5′-TAGAA-3′), and a base sequence (5′-(TAAAATAGAA)ₙ-3′, n is an integer 2 or more) consisting of multiple repeats of the base sequence of SEQ ID NO: 6 from 5′ side in this order;
(b4) a base sequence formed by linking the base sequence of SEQ ID NO: 12 (5′-(TAGAA)₂TGGAA(TAGAA)₂TGGAA-3′), a base sequence (5′-(TAGAA)ₙ-3′, n is an integer 2 or more) consisting of multiple repeats of the base sequence of SEQ ID NO: 5 (5′-TAGAA-3′), a base sequence (5′-(TAAAATAGAA)ₙ-3′, n is an integer 2 or more) consisting of multiple repeats of the base sequence of SEQ ID NO: 6, and the base sequence of SEQ ID NO: 13 (5′-(TAAAA)₃TAGAA-3′) from 5′ side in this order; and
(b5) a base sequence formed by linking the base sequence of SEQ ID NO: 14 (5′-TGGGAATAGAATGGGAA(TAGAA)₂(TGGAA)₃TAGAATGGAA-3′), a base sequence (5′-(TAGAA)ₙ-3′, n is an integer 2 or more) consisting of multiple repeats of the base sequence of SEQ ID NO: 5 (5′-TAGAA-3′), and a base sequence (5′-(TAAAATAGAA)ₙ-3′, n is an integer 2 or more) consisting of multiple repeats of the base sequence of SEQ ID NO: 6 from 5′ side in this order. Here, “n” varies depending on a case, and is not limited to the following range. Examples of “n” include an integer from 2 to 500, from 100 to 400, or from 200 to 400.

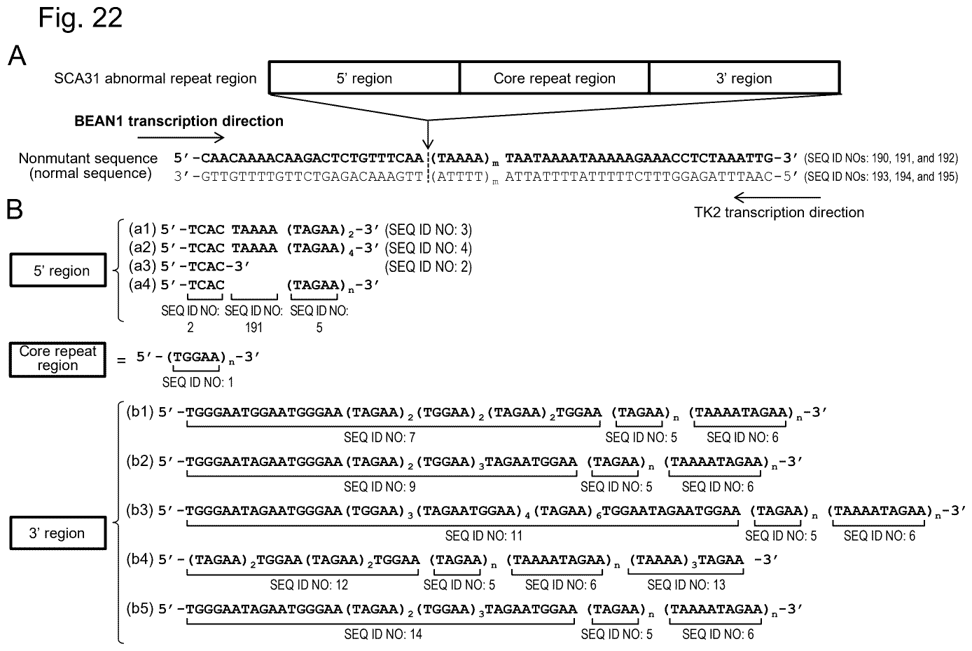

A combination of the above-described 5′ and 3′ regions in the abnormal repeat region of the mutant form of the BEAN1 gene is not particularly limited, and may be any combination. The sequences of the above-described 5′ and 3′ regions are shown in FIG. 22.

In one embodiment, a target binding region comprised by the nucleic acid molecule of the present invention can be designed to comprise a boundary position between two regions neighboring to each other in a nonmutant region neighboring to the 5′ side of the 5′ region, the 5′ region, a core repeat region, the 3′ region, and a nonmutant region neighboring to the 3′ side of the 3′ region. In other words, the target binding region comprised by the nucleic acid molecule of the present invention may comprise each of the bases that are complementary to the terminal bases of the two regions neighboring to each other in the nonmutant region neighboring to the 5′ side of the 5′ region, the 5′ region, the core repeat region, the 3′ region, and the nonmutant region neighboring to the 3′ side of the 3′ region.

Herein, a “nonmutant region” is a region that neighbors to the abnormal repeat region in the locus of a mutant form of a gene comprising an abnormal repeat region and can also be found in a normal gene. The nonmutant region may be a gene region that can be found in a normal individual (e.g., an individual suffering from neither SCA31 nor SCA10), and genetic polymorphism (e.g., single nucleotide polymorphism found in a normal individual, or polymorphism related to the number of repetitions of a repeat sequence) is permitted. The nonmutant region is either a region neighboring to the 5′ side of the 5′ region of the abnormal repeat region or a region neighboring to the 3′ side of the 3′ region of the abnormal repeat region.

In another embodiment, the full-length of a target binding region comprised by the nucleic acid molecule of the present invention is comprised by a base sequence complementary to any of a 5′ region, a core repeat region, or a 3′ region.

In one embodiment, the nucleic acid molecule of the present aspect consists of any one base sequence selected from the group consisting of SEQ ID NOs: 156 to 189 and 207 to 209, provided that “u” in the sequence may be “t”, and “t” may be “u”.

In one embodiment, a mutant gene that encodes a transcriptional product targeted by the nucleic acid molecule of the present invention is a mutant TK2 gene.

In the abnormal repeat region that can be comprised by the mutant form of the TK2 gene, the core repeat region is a base sequence complementary to a base sequence (5′-(TGGAA)ₙ-3′, n is an integer 2 or more) consisting of multiple repeats of the base sequence (5′-TGGAA-3′) of SEQ ID NO: 1, that is, a base sequence (5′-(TTCCA)ₙ-3′; n is an integer 2 or more) consisting of multiple repeats of the base sequence (5′-TTCCA-3′) of SEQ ID NO: 8. Here, “n” varies depending on a case, and is not limited to the following range. Examples of “n” include an integer from 2 to 500, from 100 to 400, or from 200 to 400.

The base sequences of the 5′ region and the 3′ region in the abnormal repeat region of the mutant form of the TK2 gene is not particularly limited as long as being a sequence that can be found in a patient with spinocerebellar ataxia of SCA31.

Examples of the 5′ region in the abnormal repeat region of the mutant form of the TK2 gene include a region comprising a sequence (5′-(TTCTATTTTA)ₙ-3′; n is an integer 2 or more) consisting of multiple repeats of the base sequence of SEQ ID NO: 16 and a sequence (5′-(TTCTA)ₙ-3′; n is an integer 2 or more) consisting of multiple repeats of the base sequence (5′-TTCTA-3′) of SEQ ID NO: 17. Specific examples of the base sequences thereof include the following (c1) to (c5):
(c1) a base sequence formed by linking a base sequence (5′-(TTCTATTTTA)ₙ-3′, n is an integer 2 or more) consisting of multiple repeats of the base sequence of SEQ ID NO: 16, a base sequence (5′-(TTCTA)˜-3′, n is an integer 2 or more) consisting of multiple repeats of the base sequence (5′-TTCTA-3′) of SEQ ID NO: 17, and the base sequence (5′-TTCCA(TTCTA)₂(TTCCA)₂(TTCTA)₂TTCCCATTCCATTCCCA-3′) of SEQ ID NO: 19 from 5′ side in this order;
(c2) a base sequence formed by linking a base sequence (5′-(TTCTATTTTA)ₙ-3′, n is an integer 2 or more) consisting of multiple repeats of the base sequence of SEQ ID NO: 16, a base sequence (5′-(TTCTA)ₙ-3′, n is an integer 2 or more) consisting of multiple repeats of the base sequence (5′-TTCTA-3′) of SEQ ID NO: 17, and the base sequence (5′-TTCCATTCTA(TTCCA)₃(TTCTA)₂TTCCCATTCTATTCCCA-3′) of SEQ ID NO: 21 from 5′ side in this order;
(c3) a base sequence formed by linking a base sequence (5′-(TTCTATTTTA)ₙ-3′, n is an integer 2 or more) consisting of multiple repeats of the base sequence of SEQ ID NO: 16, a base sequence (5′-(TTCTA)ₙ-3′, n is an integer 2 or more) consisting of multiple repeats of the base sequence (5′-TTCTA-3′) of SEQ ID NO: 17, and the base sequence (5′-TTCCATTCTATTCCA(TTCTA)₆(TTCCATTCTA)₄(TTCCA)₃TTCCCATTCTATTCCCA-3′) of SEQ ID NO: 22 from 5′ side in this order;
(c4) a base sequence formed by linking the base sequence (5′-TTCTA(TTTTA)₃-3′) of SEQ ID NO: 23, a base sequence (5′-(TTCTATTTTA)ₙ-3′, n is an integer 2 or more) consisting of multiple repeats of the base sequence of SEQ ID NO: 16, a base sequence (5′-(TTCTA)ₙ-3′, n is an integer 2 or more) consisting of multiple repeats of the base sequence (5′-TTCTA-3′) of SEQ ID NO: 17, and the base sequence (5′-TTCCA(TTCTA)₂TTCCA(TTCTA)₂-3′) of SEQ ID NO: 24 from 5′ side in this order; and
(c5) a base sequence formed by linking a base sequence (5′-(TTCTATTTTA)ₙ-3′, n is an integer 2 or more) consisting of multiple repeats of the base sequence of SEQ ID NO: 16, a base sequence (5′-(TTCTA)ₙ-3′, n is an integer 2 or more) consisting of multiple repeats of the base sequence (5′-TTCTA-3′) of SEQ ID NO: 17, and the base sequence (5′-TTCCATTCTA(TTCCA)₃(TTCTA)₂TTCCCATTCTATTCCCA-3′) of SEQ ID NO: 29 from 5′ side in this order. Here, “n” varies depending on a case, and is not limited to the following range. Examples of “n” include an integer from 2 to 500, from 100 to 400, or from 200 to 400.

Examples of the 3′ region in the abnormal repeat region of the mutant form of the TK2 gene include a region comprising the base sequence (5′-GTGA-3′) of SEQ ID NO: 26. Specific examples of the base sequences thereof include the following (d1) to (d4):
(d1) the base sequence (5′-(TTCTA)₂TTTTAGTGA-3′) of SEQ ID NO: 27,
(d2) the base sequence (5′-(TTCTA)₄TTTTAGTGA-3′) of SEQ ID NO: 28,
(d3) the base sequence (5′-GTGA-3′) of SEQ ID NO: 26, and
(d4) a base sequence formed by linking a base sequence (5′-(TTCTA)ₙ-3′; n is an integer 2 or more) consisting of multiple repeats of the base sequence (5′-TTCTA-3′) of SEQ ID NO: 17 and the base sequence (5′-GTGA-3′) of SEQ ID NO: 26 from 5′ side in this order. Here, “n” varies depending on a case, and is not limited to the following range. Examples of “n” include an integer from 2 to 500, from 100 to 400, or from 200 to 400.

A combination of the above-described 5′ and 3′ regions in the abnormal repeat region of the mutant form of the TK2 gene is not particularly limited, and may be any combination. The sequences of the above-described 5′ and 3′ regions are shown in FIG. 5.

In one embodiment, a target binding region comprised by the nucleic acid molecule of the present invention can be designed to comprise a boundary position between two regions neighboring to each other in a nonmutant region neighboring to the 5′ side of the 5′ region, the 5′ region, a core repeat region, the 3′ region, and a nonmutant region neighboring to the 3′ side of the 3′ region. In other words, the target binding region comprised by the nucleic acid molecule of the present invention may comprise each of the bases that are complementary to the terminal bases of the two regions neighboring to each other in the nonmutant region neighboring to the 5′ side of the 5′ region, the 5′ region, the core repeat region, the 3′ region, and the nonmutant region neighboring to the 3′ side of the 3′ region.

In another embodiment, the full-length of a target binding region comprised by the nucleic acid molecule of the present invention is comprised by a base sequence complementary to any of a 5′ region, a core repeat region, or a 3′ region.

In one embodiment, the nucleic acid molecule of the present aspect consists of any one base sequence selected from the group consisting of SEQ ID NOs: 33 to 53, 58 to 139, 141 to 150, 152 to 155, and 204 to 206, provided that “u” in the sequence may be “t”, and “t” may be “u”.

In one embodiment, a mutant gene that encodes a transcriptional product targeted by the nucleic acid molecule of the present invention is a mutant ATXN10 gene.

In the abnormal repeat region that can be comprised by the mutant form of the ATXN10 gene, the core repeat region is a base sequence complementary to a base sequence (5′-(TGGAA)ₙ-3′, n is an integer 2 or more) consisting of multiple repeats of the base sequence (5′-TGGAA-3′) of SEQ ID NO: 1, that is, a base sequence (5′-(TTCCA)ₙ-3′; n is an integer 2 or more) consisting of multiple repeats of the base sequence (5′-TTCCA-3′) of SEQ ID NO: 8. Here, “n” varies depending on a case, and is not limited to the following range. Examples of “n” include an integer from 2 to 600, from 100 to 500, or from 200 to 400.

The base sequences of the 5′ region and the 3′ region in the abnormal repeat region of the mutant form of the ATXN10 gene is not particularly limited as long as being a sequence that can be found in a patient with spinocerebellar ataxia of SCA0. Specific examples of the 5′ region in the abnormal repeat region of the mutant form of the ATXN10 gene include, for example, a region comprising the base sequence (5′-TTCTA-3′) of SEQ ID NO: 17 or a base sequence (5′-(TTCTA)ₙ-3′, n is an integer 2 or more) consisting of multiple repeats thereof (n is an integer 2 or more, varies depending on a case, and is not limited to the following range, and examples thereof include an integer from 2 to 600, from 100 to 500, or from 200 to 400). Specific examples of the 3′ region in the abnormal repeat region of the mutant form of the ATXN10 gene include, for example, a region comprising the base sequence (5′-TTCTA-3′) of SEQ ID NO: 17 or a base sequence (5′-(TTCTA)ₙ-3′, n is an integer 2 or more) consisting of multiple repeats thereof (n is an integer 2 or more, varies depending on a case, and is not limited to the following range, and examples thereof include an integer 100 or more, 200 or more, or 400 or more).

In one embodiment, a target binding region comprised by the nucleic acid molecule of the present invention can be designed to comprise a boundary position between two regions neighboring to each other in a nonmutant region neighboring to the 5′ side of the 5′ region, the 5′ region, a core repeat region, the 3′ region, and a nonmutant region neighboring to the 3′ side of the 3′ region. In other words, the target binding region comprised by the nucleic acid molecule of the present invention may comprise each of the bases that are complementary to the terminal bases of the two regions neighboring to each other in the nonmutant region neighboring to the 5′ side of the 5′ region, the 5′ region, the core repeat region, the 3′ region, and the nonmutant region neighboring to the 3′ side of the 3′ region.

In another embodiment, the full-length of a target binding region comprised by the nucleic acid molecule of the present invention is comprised by a base sequence complementary to any of a 5′ region, a core repeat region, or a 3′ region.

In one embodiment, the nucleic acid molecule of the present aspect consists of any one base sequence selected from the group consisting of SEQ ID NOs: 33 to 37, 58 to 61, 64 to 67, 70 to 139, 141 to 150, 152 to 155, and 204 to 206, provided that “u” in the sequence may be “t”, and “t” may be “u”.

There is no particular restriction on the base length of the nucleic acid molecule of the present invention, and the base length may be at least 8 base in length, at least 9 base in length, at least 10 base in length, at least 11 base in length, at least 12 base in length, at least 13 base in length, at least 14 base in length, or at least 15 base in length. Further, the base length of the nucleic acid molecule may be 40 base in length or less, 35 base in length or less, 30 base in length or less, 25 base in length or less, 24 base in length or less, 23 base in length or less, 22 base in length or less, 21 base in length or less, 20 base in length or less, 19 base in length or less, 18 base in length or less, 17 base in length or less, or 16 base in length or less. The base length of the nucleic acid molecule may be, for example, 10 to 40 base in length, 12 to 30 base in length, or 15 to 25 base in length. For the selection of the length, the length can be determined according to the balance between the strength of the antisense effect and the specificity of the nucleic acid strand to the target, among other factors such as cost, and synthesis yield. When a nucleic acid such as an aptamer is bound to the nucleic acid molecule, the base length of the nucleic acid molecule as a whole may be the above-described base length plus the base length of the bound nucleic acid.

A nucleoside comprised by the nucleic acid molecule of the present invention may be a natural nucleoside (deoxyribonucleoside, ribonucleoside, or both) and/or a non-natural nucleoside.

The nucleic acid molecule of the present invention may be a mixmer. The term “mixmer” refers to herein a nucleic acid strand that comprises natural nucleosides and non-natural nucleosides with periodically or randomly alternating segment lengths, and does not comprise four or more consecutive deoxyribonucleosides or ribonucleosides. Among mixmers, a mixmer in which said non-natural nucleoside is a bridged nucleoside, and the natural nucleoside is a deoxyribonucleoside is specifically referred to as a “BNA/DNA mixmer”. Among mixmers, a mixmer in which said non-natural nucleoside is a peptide nucleic acid and the natural nucleoside is a deoxyribonucleoside is specifically called a “peptide nucleic acid/DNA mixmer”. Among mixmers, a mixmer in which said non-natural nucleoside is a morpholino nucleic acid, and the natural nucleoside is a deoxyribonucleoside is specifically referred to as a “morpholino nucleic acid/DNA mixmer”. A mixmer is not restricted to comprise only two kinds of nucleosides. A mixmer may comprise any number of kinds of nucleosides irrespective of a natural or modified nucleoside, or a nucleoside mimic. For example, a mixmer may comprise one or two consecutive deoxyribonucleosides separated by a bridged nucleoside (e.g., LNA nucleoside). A bridged nucleoside may further comprise a modified nucleobase (e.g., 5-methylcytosine).

The nucleic acid molecule of the present invention may be a gapmer. Herein, a “gapmer” refers to, as a general rule, a single-stranded nucleic acid consisting of a “central region” (DNA gap region) and wing regions directly positioned at the 5′-end and 3′-end thereof (referred to as “5′-wing region” and “3′-wing region”, respectively). Said central region in the gapmer comprises at least two (e.g., at least three or at least four) consecutive deoxyribonucleosides (that may comprise a modified nucleobase that is recognized by RNase H, or may comprise, for example, 5-methylcytosine), and said wing regions comprise at least one non-natural nucleoside. Without limitation, the non-natural nucleoside comprised by the wing regions typically has the higher power of binding to RNA is than a natural nucleoside, and has high resistance to a nucleic acid-degrading enzyme (a nuclease or the like). The non-natural nucleoside constituting the 5′-wing region and the 3′-wing region may be, for example, bridged nucleosides and/or 2′-modified nucleosides. When the non-natural nucleoside constituting the wing regions comprises or consists of the bridged nucleosides, said gapmer is specifically referred to as a “BNA/DNA gapmer”. The number of the bridged nucleosides comprised by the 5′-wing region and the 3-wing region is at least one, and may be, for example, two or three. The bridged nucleosides comprised by the 5′-wing region and the 3′-wing region may consecutively or disconsecutively exist in the 5′-wing region and the 3′-wing region. The bridged nucleosides may further comprise a modified nucleobase (e.g., 5-methylcytosine). The bridged nucleosides may be LNA nucleosides or ENA nucleosides. When the bridged nucleosides are the LNA nucleosides, said gapmer is referred to as an “LNA/DNA gapmer”. When the bridged nucleosides are the ENA nucleosides, said gapmer is referred to as an “ENA/DNA gapmer”. When the non-natural nucleoside constituting the 5′-wing region and the 3′-wing region comprises or consists of a peptide nucleic acid, said gapmer is specifically referred to as a “peptide nucleic acid gapmer”. When the non-natural nucleoside constituting the 5′-wing region and the 3′-wing region comprises or consists of a morpholino nucleic acid, said gapmer is specifically referred to as a “morpholino nucleic acid gapmer”. When the non-natural nucleoside constituting the 5′-wing region and the 3′-wing region comprises or consists of 2′-modified nucleosides, the 2′-modification groups of the 2′-modified nucleosides may be a 2′-O-methyl group or a 2′-O-methoxyethyl group. The number of the 2′-modified nucleosides comprised by the 5′-wing region and the 3′-wing region is at least one, and may be, for example, two or three. The 2′-modified nucleosides comprised by the 5′-wing region and the 3′-wing region may consecutively or disconsecutively exist in the 5′-wing region and the 3′-wing region. The 2′-modified nucleosides may further comprise a modified nucleobase (e.g., 5-methylcytosine). When the non-natural nucleoside constituting the 5′-wing region and the 3′-wing region comprises or consists of bridged nucleosides and 2′-modified nucleosides, two or more kinds of bridged nucleosides and/or 2′-modified nucleosides may be combined and composed of. For example, a combination of two kinds thereof may be LNA nucleoside and ENA nucleoside; LNA nucleoside and 2′-O-methylnucleoside; LNA nucleoside and 2′-O-methoxyethylnucleoside; ENA nucleoside and 2′-O-methylnucleoside; ENA nucleoside and 2′-0-methoxyethylnucleoside; or 2′-O-methylnucleoside and 2′-O-methoxyethylnucleoside. For example, a combination of three kinds thereof may be LNA nucleoside, ENA nucleoside, and 2′-O-methylnucleoside; LNA nucleoside, ENA nucleoside, and 2′-O-methoxyethylnucleoside; or ENA nucleoside, 2′-O-methylnucleoside, and 2′-O-methoxyethylnucleoside. For example, a combination of four kinds thereof may be LNA nucleoside, ENA nucleoside, 2′-O-methylnucleoside, and 2′-O-methoxyethylnucleoside.

When the nucleic acid molecule of the present invention is a gapmer, the DNA gap region may be, for example, 4 to 10 base in length, 5 to 8 base in length, 5 to 8 base in length, 6 to 8 base in length, 7 base in length, or 8 base in length. The DNA gap region is composed of a natural nucleoside consisting of DNA.

When the nucleic acid molecule of the present invention is a gapmer, the base lengths of the 5′-wing region and 3′-wing region of the gapmer may be each independently at least 2 base in length, for example, 2 to 10 base in length, 2 to 7 base in length, 3 to 5 base in length, 3 to 4 base in length, or 3 base in length. The nucleic acid molecule of the present invention may comprise at least one LNA or ENA in the 5′-wing region and the 3′-wing region. For example, the 5′-wing region may comprise at least one, for example, one to four, two to four, and two to three, for example, two LNAs or ENAs. The 3′-wing region may comprise at least one, for example, one to four, two to four, and two to three, for example, two LNAs or ENAs. The kinds, numbers, and positions of the modifications of the 5′-wing region and the 3′-wing region may be the same or different. In the 5′-wing region and the 3′-wing region, 2′-O-methylnucleoside, 2′-O-methoxyethylnucleoside, 2′-LNA, or ENA may be combined, the kinds of the modifications thereof may be one to four kinds, or two to three kinds, for example, two kinds, and the kinds may be the same or different in the 5′-wing region and the 3′-wing region.

When the nucleic acid molecule of the present invention is a gapmer, examples of the base length of each region of the 5′-wing region, the DNA gap region, and the 3′-wing region include 2-8-3, 3-8-2, 3-7-3, 4-6-3, 3-6-4, 4-5-4, 4-7-3, 3-7-4, 4-6-4, 5-6-3, 3-6-5, 3-7-5, 5-7-3, 4-7-4, 4-6-5, 5-6-4, 5-5-5, and 5-6-5. Here, in the expression of “A-B-C”, “A” shows the base length of the 5′-wing region, “B” shows the base length of the DNA gap region, and “C” shows the base length of the 3′-wing region.

A nucleic acid strand having a wing region only in either a 5′-end side or a 3′-end side is referred to as a “hemi-gapmer” in the art. However, herein, a hemi-gapmer is also encompassed by a gapmer.

The internucleoside linkage in the nucleic acid molecule of the present invention may be a naturally occurring internucleoside linkage and/or a modified internucleoside linkage. Without limitation, at least one, at least two, or at least three internucleoside linkages from an end (5′ end, 3′ end or both the ends) of the nucleic acid molecule of the present invention are preferably modified internucleoside linkages. In this regard, for example, two internucleoside linkages from the end of a nucleic acid strand refers to an internucleoside linkage closest to the end of the nucleic acid strand, and an internucleoside linkage positioned next thereto on the opposite side to the end of the nucleic acid strand. Modified internucleoside linkages in the terminal region of a nucleic acid strand are preferred because they can reduce or inhibit undesired degradation of the nucleic acid strand. In one embodiment, all or some of the internucleoside linkages of the nucleic acid molecule may be modified internucleoside linkages. The modified internucleoside linkage may be a phosphorothioate linkage.

The nucleic acid molecule of the present invention may comprise, as a whole or in part, a nucleoside mimic or a nucleotide mimic. The nucleotide mimic may be a peptide nucleic acid and/or a morpholino nucleic acid. In one embodiment, the nucleic acid molecule of the present invention may comprise or consists of a morpholino nucleic acid.

In one embodiment, natural ribonucleosides of the nucleic acid molecule of the present invention are half of or less than half of the total length of the nucleic acid molecule, or are not comprised.

In one embodiment, the nucleic acid molecule of the present invention may comprise a modified nucleobase. The number of such modified nucleobases is not limited, and may be, for example, at least one, at least two, at least three, at least four, at least five, or at least six.

The antisense effect on target transcriptional product of the nucleic acid molecule of the present invention can be measured by a method known in the art. For example, after introducing a nucleic acid molecule into a cell and the like, it can be measured using a publicly known technique such as Northern blotting, quantitative PCR, or Western blotting. By measuring the expression level of a target gene or the level of a target transcriptional product in specific tissues (e.g., the brain) (e.g., the amount of RNA, such as the amount of mRNA, the amount of cDNA, or the like), it can be judged whether or not the target gene expression is suppressed by the nucleic acid molecule in these sites. When the measured expression level of the target gene or the measured level of the target transcriptional product is at least 20%, at least 25%, at least 30%, at least 40%, or at least 50% less than that of a negative control (e.g., administration of a vehicle, or no treatment), it is shown that the test nucleic acid compound can produce an antisense effect.

### <Double-Stranded Nucleic Acid Complex>

In one aspect, the present invention relates to a double-stranded nucleic acid complex. The double-stranded nucleic acid complex of the present invention comprises a first nucleic acid strand and a second nucleic acid strand. In the double-stranded nucleic acid complex of the present invention, the first nucleic acid strand can function as ASO. The specific configuration of each nucleic acid strand is described below.

The first nucleic acid strand can be selected from the embodiments of the nucleic acid molecule described above. For example, the first nucleic acid strand consists of the above-described nucleic acid molecule which is a gapmer; or the above-described nucleic acid molecule comprising (1) a central region comprising at least two (e.g., at least three or at least four) consecutive deoxyribonucleosides, (2) a 5′-wing region comprising a non-natural nucleoside, positioned in the 5′-end side of said central region, and (3) a 3′-wing region comprising a non-natural nucleoside, positioned in the 3′-end side of said central region. The gapmer and the configurations (1) to (3) described above conform to the description related to the nucleic acid molecule described above.

The second nucleic acid strand is a nucleic acid molecule comprising a base sequence complementary to the first nucleic acid strand. In the double-stranded nucleic acid complex of the present invention, the second nucleic acid strand is annealed to the first nucleic acid strand via hydrogen bonds of complementary base pairs. As an example of the present embodiment, there is a heteroduplex oligonucleotide (HDO) disclosed in WO2013/089283, Nishina K, et. al, Nature Communication, 2015, 6:7969, and Asami Y, et al, Drug Discoveries &Therapeutics. 2016; 10(5):256-262.

In one embodiment, the double-stranded nucleic acid complex of the present invention comprises any first nucleic acid strand selected from the embodiments of the nucleic acid molecule described above and a second nucleic acid strand comprising a base sequence complementary to the first nucleic acid strand. In a further embodiment, the second nucleic acid strand may comprise a ribonucleoside, a deoxyribonucleoside, and/or a modified nucleoside.

In one embodiment, in the second nucleic acid strand, all of the nucleosides in the region consisting of the base sequence complementary to the central region of the first nucleic acid strand may be: (a) deoxyribonucleosides; (b) deoxyribonucleosides and ribonucleosides; (c) deoxyribonucleosides and 2′-modified nucleosides; (d) ribonucleosides and 2′-modified nucleosides; or (e) deoxyribonucleosides, ribonucleosides, and 2′-modified nucleosides.

In one embodiment, the second nucleic acid strand comprises a region comprising at least two consecutive ribonucleosides and/or deoxyribonucleosides which are complementary to at least two consecutive deoxyribonucleosides in the central region in the first nucleic acid strand. For example, the second nucleic acid strand may comprise a region comprising at least three or at least four consecutive ribonucleosides and/or deoxyribonucleosides complementary to at least three or at least four consecutive deoxyribonucleosides in the central region of the first nucleic acid strand. Here, the number of the consecutive deoxyribonucleosides in the central region of the first nucleic acid strand and the number of the consecutive ribonucleosides and/or deoxyribonucleosides complementary to the consecutive deoxyribonucleosides in the second nucleic acid strand may be the same or different. For example, the number of the consecutive deoxyribonucleosides described above may be at least four, and the number of the consecutive ribonucleosides and/or deoxyribonucleosides described above may be at least three.

In a further embodiment, the second nucleic acid strand may comprise a region consisting of a base sequence complementary to the 5′-wing region and/or 3-wing region of the first nucleic acid strand. In the second nucleic acid strand, the region consisting of the base sequence complementary to the 5′-wing region and/or 3′-wing region of the first nucleic acid strand may comprise at least one non-natural nucleoside, and the non-natural nucleoside may be, for example, a bridged nucleoside and/or a 2′-modified nucleoside. In a further embodiment, the bridged nucleoside in the second nucleic acid strand may be an LNA nucleoside, an ENA nucleoside, or a BNA^{NC} nucleoside. The 2′-modification group of the 2′-modified nucleoside in the second nucleic acid strand may be a 2′-O-methyl group or a 2′-O-methoxyethyl group. When both the first nucleic acid strand and the second nucleic acid strand comprise bridged nucleosides and/or 2′-modified nucleosides, the bridged nucleosides and/or the 2′-modified nucleosides in the first nucleic acid strand and the second nucleic acid strand may be the same or different.

The internucleoside linkage in the second nucleic acid strand may be a naturally occurring internucleoside linkage and/or a modified internucleoside linkage. Without limitation, at least one, at least two, or at least three internucleoside linkages from an end (5′ end, 3′ end, or both ends) of the second nucleic acid strand are preferably modified internucleoside linkages. In one embodiment, all or some of the internucleoside linkages of the second nucleic acid strand may be modified internucleoside linkages. In one embodiment, the second nucleic acid strand may comprise a modified internucleoside linkage in a region consisting of a base sequence complementary to the 5′-wing region and/or 3′-wing region of the first nucleic acid strand. The modified internucleoside linkage may be a phosphorothioate linkage.

In a further embodiment, the second nucleic acid strand can comprise 2′-O-methoxyethyl-modified nucleosides. In the second nucleic acid strand, the number of the 2′-0-methoxyethyl-modified nucleosides is not limited. For example, at least 20%, at least 30%, at least 40%, at least 50%, at least 60%, at least 70%, at least 75%, at least 80%, at least 85%, at least 90%, or at least 95%, or 100% of the total number of nucleosides in said second nucleic acid strand may be the 2′-O-methoxyethyl-modified nucleosides. In one embodiment, all of the nucleosides in the second nucleic acid strand are the 2′-O-methoxyethyl-modified nucleosides.

In a further embodiment, the second nucleic acid strand can comprise one or two or more consecutive 2′-O-methoxyethyl-modified nucleosides positioned at the 5′-end and/or one or two or more consecutive 2′-O-methoxyethyl-modified nucleosides positioned at the 3′-end. The number of the 2′-O-methoxyethyl-modified nucleosides positioned at the 5′-end and/or the 3′-end is not limited. For example, the second nucleic acid strand may comprise one or two, three, four, five, six, or seven consecutive 2′-O-methoxyethyl-modified nucleosides positioned at the 5′-end and/or one or two, three, four, five, six, or seven consecutive 2′-O-methoxyethyl-modified nucleosides positioned at the 3′-end.

The second nucleic acid strand may comprise one to seven 2′-O-methoxyethyl-modified nucleosides at positions except one or two or more consecutive 2′-O-methoxyethyl-modified nucleosides positioned at the 5′-end and/or 3′-end described above.

In any of the embodiments described above, nucleosides except the 2′-O-methoxyethyl-modified nucleosides are not particularly limited in the second nucleic acid strand. For example, all the nucleosides except the 2′-O-methoxyethyl-modified nucleosides may be deoxyribonucleosides in the second nucleic acid strand.

In one embodiment, the second nucleic acid strand may comprise modified nucleobases. The number of the modified nucleobases is not limited, and may be, for example, at least one, at least two, at least three, at least four, at least five, or at least six.

In one embodiment, the second nucleic acid strand can further comprise at least one overhang region located on one or both the 5′ end side and the 3′ end side of the complementary region. An example of the present embodiment is described in WO2018/062510. The term “overhang region” refers to a region adjacent to a complementary region, namely a nucleotide region in the second nucleotide strand in which the 5′ end of the second nucleotide strand extends beyond the 3′ end of the first nucleotide strand and/or the 3′ end of the second nucleotide strand extends beyond the 5′ end of the first nucleotide strand when the first nucleic acid strand and the second nucleic acid strand are annealed to form a double-stranded structure, or protruding from the double-stranded structure. The overhang region in the second nucleic acid strand may be located at the 5′ end side or 3′ end side of the complementary region. The overhang region in the second nucleic acid strand may be located at the 5′ end side and 3′-end side of the complementary region.

In one embodiment, a functional moiety may be bound to the first nucleic acid strand and/or the second nucleic acid strand, e.g., the second nucleic acid strand. The linkage between the first nucleic acid strand and/or the second nucleic acid strand and the functional moiety may be a direct linkage or an indirect linkage via another substance, and in some embodiments, it is preferable that the first nucleic acid strand and/or the second nucleic acid strand and the functional moiety are directly bound by covalent bonding, ionic bonding, hydrogen bonding, or the like, and from the viewpoint of obtaining a more stable bond, covalent bonding is more preferable.

In some embodiments, the structure of the “functional moiety” is not particularly restricted and imparts a desired function on a double-stranded nucleic acid complex to which it is bound. Examples of the desired function include a labeling function, a purification function, and a delivery function to a target. Examples of a moiety that provides a labeling function include compounds such as a fluorescent protein, and luciferase. Examples of a moiety that provides a purifying function include compounds such as biotin, avidin, His-tag peptide, GST-tag peptide, and FLAG-tag peptide. A molecule having an activity for delivery of a double-stranded nucleic acid complex in an embodiment to a target site is preferably bound as a functional moiety to the first nucleic acid strand and/or the second nucleic acid strand, from the viewpoint that the first nucleic acid strand is efficiently delivered to a target site at a high specificity and expression of a target gene is very effectively suppressed by the nucleic acid. Examples of a moiety for providing a delivery function to a target include lipid, antibody, aptamer, and a ligand to a particular receptor.

In one embodiment, the first nucleic acid strand and/or the second nucleic acid strand, for example, the second nucleic acid strand is bound to a lipid. Examples of a lipid include, but not limited to, tocopherol, cholesterol, fatty acid, phospholipid, and analogs thereof; folic acid, vitamin C, vitamin B1, vitamin B2; estradiol, androstane, and analogs thereof; steroid and an analog thereof; ligand of LDLR, SRBI or LRP1/2; FK-506, and cyclosporine; lipids described in PCT/JP2019/12077 and PCT/JP2019/10392. A lipid may be a tocopherol or an analog thereof and/or a cholesterol or an analog thereof, a substituted or unsubstituted C₁₋₃₀ alkyl group, a substituted or unsubstituted C₂₋₃₀ alkenyl group, or a substituted or unsubstituted C₁₋₃₀ alkoxy group. In one embodiment, the second nucleic acid strand bound to tocopherol or cholesterol, or an analog thereof.

The functional moiety may be linked to the 5′ end, or the 3′ end, or both ends of the first nucleic acid strand and/or the second nucleic acid strand. Alternatively, the functional moiety may be linked to a nucleotide in the interior part of the first nucleic acid strand and/or the second nucleic acid strand. The first nucleic acid strand and/or the second nucleic acid strand may comprise two or more functional moieties, such as lipids, which may be linked to a plurality of positions in the first nucleic acid strand and/or the second nucleic acid strand, and/or linked as a group to a single position in the first nucleic acid strand and/or the second nucleic acid strand. Functional moieties may be linked to the first nucleic acid strand and/or the second nucleic acid strand, one at the 5′ end and one at the 3′ end of the first nucleic acid strand.

The linkage between the first nucleic acid strand and/or the second nucleic acid strand and a functional moiety may be a direct linkage or an indirect linkage that is mediated by another substance. However, in a specific embodiment, a functional moiety is preferably directly bound to the first nucleic acid strand and/or the second nucleic acid strand via covalent bonding, ionic bonding, hydrogen bonding, or the like, and from the viewpoint of obtaining a more stable bond, covalent bonding is more preferable.

The functional moiety may also be bound to the first nucleic acid strand and/or the second nucleic acid strand via a cleavable or uncleavable linker. In this case, the first nucleic acid strand and the second nucleic acid strand can be linked via a linker to form a single-strand. However, even in that case, the functional region has the same configuration as the double-stranded nucleic acid complex, and therefore such a single-stranded nucleic acid is herein also encompassed as an embodiment of the double-stranded nucleic acid complex of the present invention. The linker can be any polymer. Examples thereof include a polynucleotide, polypeptide, and alkylene. Specifically, it can be composed of a natural nucleotide such as DNA, and RNA, or a non-natural nucleotide such as a peptide nucleic acid and a morpholino nucleic acid. When a linker consists of a nucleic acid, the chain length of a linker may be at least one base, or a chain length of from 3 to 10 bases or from 4 to 6 bases. It is preferably 4 base in length. In this case, the linker can take the form of a hinge (hairpin loop). The position of the linker can be either on the 5′ side or the 3′ side of the first nucleic acid strand. For example, in the case of a configuration in which the second nucleic acid strand is bound to the 5′ side of the first nucleic acid strand, the 5′ end of the first nucleic acid strand and the 3′ end of the second nucleic acid strand are linked via a linker.

A “cleavable linker” means a linkage group that is cleaved under physiological conditions, e.g., in a cell or in an animal body (e.g., in a human body). In a specific embodiment, a cleavable linker is selectively cleaved by an endogenous enzyme such as a nuclease. Examples of a cleavable linker comprise an amide, an ester, one or both esters of a phosphodiester, a phosphoester, a carbamate, and a disulfide bond, as well as a natural DNA linker.

An “uncleavable linker” refers to a linker that is not cleaved under physiological conditions, for example, in a cell or in an animal body (e.g., in a human body). Examples of an uncleavable linker comprise, but not limited to, a phosphorothioate linkage, modified or unmodified deoxyribonucleosides linked by a phosphorothioate linkage, and a linker consisting of modified or unmodified ribonucleosides. There is no particular restriction on the chain length, when a linker is a nucleic acid such as DNA, or an oligonucleotide, however it may be usually from 2 to 20 base in length, from 3 to 10 base in length, or from 4 to 6 base in length.

Specific examples of the linker comprise a linker represented by the following Formula (I).

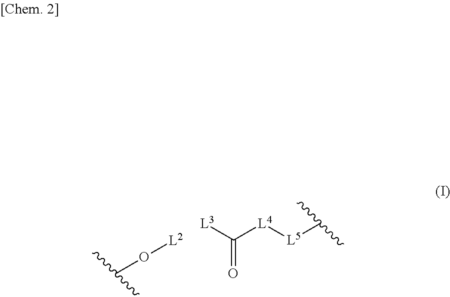

(where L² represents a substituted or unsubstituted C₁-C₁₂ alkylene group (for example, propylene, hexylene, dodecylene), a substituted or unsubstituted C₃-C₈ cycloalkylene group (for example, cyclohexylene), —(CH₂)₂—O—(CH₂)₂—O—(CH₂)₂—O—(CH₂)₃—, —(CH₂)₂—O—(CH₂)₂—O—(CH₂)₂—O—(CH₂)₂—O—(CH₂)₃—, or CH(CH₂—OH)—CH₂—O—(CH₂)₂—O—(CH₂)₂—O—(CH₂)₂—O—(CH₂)₃—, L³ represents —NH— or a bond, L⁴ represents a substituted or unsubstituted C₁-C₁₂ alkylene group (for example, ethylene, pentylene, heptylene, or undecylene), a substituted or unsubstituted C₃-C₈ cycloalkylene group (for example, cyclohexylene), —(CH₂)₂—[O—(CH₂)₂]ₘ—, or a bond, wherein m is an integer from 1 to 25, L⁵ represents —NH—(C═O)—, —(C═O)—, or a bond (here, the substitution is preferably carried out by a halogen atom).

In one embodiment, regarding a linker represented by Formula (I), L² is unsubstituted C₃-C₆ alkylene group (for example, propylene, hexylene), —(CH₂)₂—O—(CH₂)₂—O—(CH₂)₂—O—(CH₂)₃—, or —(CH₂)₂—O—(CH₂)₂—O—(CH₂)₂—O—(CH₂)₂—O—(CH₂)₃—, L³ is —NH—, and L⁴ and L⁵ are bonds.

There is no particular restriction on the base lengths of the first nucleic acid strand and the second nucleic acid strand, and may be at least 8 base in length, at least 9 base in length, at least 10 base in length, at least 11 base in length, at least 12 base in length, at least 13 base in length, at least 14 base in length, or at least 15 base in length. Further, the base length of the first nucleic acid strand and the second nucleic acid strand may be 40 base in length or less, 35 base in length or less, 30 base in length or less, 25 base in length or less, 24 base in length or less, 23 base in length or less, 22 base in length or less, 21 base in length or less, 20 base in length or less, 19 base in length or less, 18 base in length or less, 17 base in length or less, or 16 base in length or less. The first nucleic acid strand and the second nucleic acid strand may have the same length or different lengths (for example, one of them is shorter or longer by 1 to 3 bases). The double-stranded structure formed by the first nucleic acid strand and the second nucleic acid strand may comprise a bulge. The length can be determined according to the balance between the strength of the antisense effect and the specificity of the nucleic acid strand to the target, among other factors such as cost, and synthesis yield. When a nucleic acid such as an aptamer is bound to the first nucleic acid strand and/or the second nucleic acid strand, the base length of the first nucleic acid strand and the second nucleic acid strand as a whole may be the above-described base length plus the base length of the bound nucleic acid. In this case, the base length of the bound nucleic acid is not limited, and may be, for example, at least 10 base in length, at least 15 base in length, or at least 20 base in length, and may be 100 base in length or less, 80 base in length or less, 60 base in length or less, 40 base in length or less, or 30 base in length or less.

In one embodiment, the mutant gene that encodes the transcriptional product targeted by the double-stranded nucleic acid complex of the present invention is a mutant TK2 gene, the base sequence of the first nucleic acid strand consists of the base sequence of SEQ ID NO: 65, provided that “t” in the sequence may be “u”, and the base sequence of the second nucleic acid strand consists of the base sequence of SEQ ID NO: 140, SEQ ID NO: 200, SEQ ID NO: 201, SEQ ID NO: 202, or SEQ ID NO: 203, provided that “t” in the sequence may be “u”. As long as the base sequence of the first nucleic acid strand in the present embodiment consists of the base sequence of SEQ ID NO: 65, provided that “t” in the sequence may be “u”, glycosylation and internucleoside modification in the nucleic acid strand are not particularly limited. For example, all or some of internucleoside linkages may be modified internucleoside linkages, and the modified internucleoside linkages may be phosphorothioate linkages. The same applies to the second nucleic acid strand.

In a further embodiment, the mutant gene that encodes the transcriptional product targeted by the double-stranded nucleic acid complex of the present invention is a mutant TK2 gene, the first nucleic acid strand consists of the nucleic acid strand of SEQ ID NO: 65, and the second nucleic acid strand consists of the nucleic acid strand of SEQ ID NO: 140, SEQ ID NO: 200, SEQ ID NO: 201, SEQ ID NO: 202, or SEQ ID NO: 203. All of the base sequence, glycosylation, and internucleoside linkage of the first nucleic acid strand in the present embodiment are specified as the structure of SEQ ID NO: 65. The same applies to the second nucleic acid strand.

### <Pharmaceutical Composition>

In one aspect, the present invention relates to a pharmaceutical composition. The pharmaceutical composition of the present invention comprises, as an active ingredient, the nucleic acid molecule or double-stranded nucleic acid complex described herein, and can be used for treating spinocerebellar ataxia type 31 (SCA31) or spinocerebellar ataxia type 10 (SCA10).

### (Active Ingredient)

The pharmaceutical composition of the present invention may comprise two kinds or more of the nucleic acid molecule and/or double-stranded nucleic acid complex described herein.

In one embodiment, a pharmaceutical composition for treating spinocerebellar ataxia type 31 (SCA31), comprising a nucleic acid molecule and/or a double-stranded nucleic acid complex targeting the transcriptional product of a mutant BEAN1 gene, is provided as the pharmaceutical composition of the present invention.

In one embodiment, a pharmaceutical composition for treating spinocerebellar ataxia type 31 (SCA31), comprising a nucleic acid molecule and/or a double-stranded nucleic acid complex targeting the transcriptional product of a mutant TK2 gene, is provided as the pharmaceutical composition of the present invention.

In one embodiment, a pharmaceutical composition for treating spinocerebellar ataxia type 31 (SCA31), comprising a nucleic acid molecule and/or a double-stranded nucleic acid complex targeting the transcriptional product of a mutant BEAN1 gene and a nucleic acid molecule and/or a double-stranded nucleic acid complex targeting the transcriptional product of a mutant TK2 gene, is provided as the pharmaceutical composition of the present invention.

In one embodiment, a pharmaceutical composition for treating spinocerebellar ataxia type 10 (SCA10), comprising a nucleic acid molecule and/or a double-stranded nucleic acid complex targeting the transcriptional product of a mutant ATXN10 gene, is provided as the pharmaceutical composition of the present invention.

The amount (content) of the nucleic acid molecule or double-stranded nucleic acid complex comprised by the pharmaceutical composition varies depending on the kind of the nucleic acid molecule of double-stranded nucleic acid complex, the site (e.g., brain) to be delivered, the dosage form of the pharmaceutical composition, the dose of the pharmaceutical composition, and the kind of carrier described below. Therefore, it may be determined as appropriate by taking the respective conditions into consideration. Usually, it may be adjusted so that an effective amount of the nucleic acid molecule or the double-stranded nucleic acid complex is contained in a single dose of the pharmaceutical composition. An “effective amount” may be an amount that is necessary for the nucleic acid molecule or the double-stranded nucleic acid complex to function as an active ingredient. An “effective amount” may be those having little or no adverse side effects on the living body to which it is applied. This effective amount can vary depending on various conditions such as information on the subject, the administration route, and the number of administrations. Ultimately, it may be determined by the judgment of a physician, veterinarian, pharmacist, or the like. “Information on the subject” is various information on an individual of the living body to which the pharmaceutical composition is applied. For example, when the subject is a human, it comprises age, body weight, gender, dietary habit, health status, stage of progression or grade of severity of the disease, drug sensitivity, and presence of a combined drug.

The pharmaceutical composition of the present invention may consist solely of the nucleic acid molecule or double-stranded nucleic acid complex described herein. Alternatively, the pharmaceutical composition of the present invention may further comprise an auxiliary component such as a carrier in addition to the nucleic acid molecule or double-stranded nucleic acid complex described herein.

### (Carrier)

The pharmaceutical composition of the present invention may comprise a pharmaceutically acceptable carrier. A “pharmaceutically acceptable carrier” refers to an additive commonly used in the field of pharmaceutical preparation. Examples thereof include a solvent, a vegetable oil, a base, an emulsifier, a suspending agent, a surfactant, a pH adjuster, a stabilizer, a seasoning, a flavor, an excipient, a vehicle, a preservative, a binder, a diluent, an isotonizing agent, a sedative, a bulking agent, a disintegrating agent, a buffering agent, a coating agent, a lubricant, a colorant, a sweetener, a thickener, a corrective agent, a dissolution aid, and other additives.

The solvent may be any of, for example, water or other pharmaceutically acceptable aqueous solution, and a pharmaceutically acceptable organic solvent. Examples of an aqueous solution comprise a physiological saline, an isotonic solution containing glucose or another additive, a phosphate-buffered saline, and a sodium acetate buffer solution. Examples of the additive comprise D-sorbitol, D-mannose, D-mannitol, sodium chloride, and further a nonionic surfactant at a low concentration, and polyoxyethylene sorbitan fatty acid ester.

The above carrier is used to avoid or decrease degradation of the nucleic acid molecule or the double-stranded nucleic acid complex, which is an active ingredient, in vivo by an enzyme and the like, and additionally to facilitate formulation or administration, and to maintain the dosage form and drug efficacy. Therefore, it may be used as appropriate and as needed.

### (Dosage Form)

There is no particular restriction on the dosage form of the pharmaceutical composition of the present invention as long as the nucleic acid molecule or double-stranded nucleic acid complex described herein, which is an active ingredient, is not inactivated by degradation or the like, and the pharmacological effect of the active ingredient can be produced in vivo.

The specific dosage form varies depending on the administration method and/or medication conditions. The administration methods can be broadly classified into parenteral administration and peroral administration, and the dosage form appropriate for the respective administration methods can be selected.

When the administration method is parenteral administration, the preferred dosage form is liquid formulation which can be administered directly to the target site, or administered systemically via the circulatory system. Examples of the liquid formulation comprise an injectable. An injectable can be formulated by mixing in an appropriate combination with said excipient, elixir, emulsifier, suspending agent, surfactant, stabilizer, pH adjuster, etc. in the form of a unit dose required according to the generally approved pharmaceutical practices. In addition, it may be ointment, plaster, cataplasm, transdermal patch, lotion, inhalant, aerosol, eye drop, and suppository.

When the administration method is oral administration, the preferred dosage form comprises solid preparation (comprising tablet, capsule, drop, and lozenge), granule, dusting powder, powder, and liquid formulation (comprising oral liquid preparation, emulsion formulation, and syrup). In the case of a solid preparation, if necessary, it may take a dosage form with a coating as publicly known in the art, such as a sugar-coated tablet, a gelatin-coated tablet, an enteric-coated tablet, a film-coated tablets, a double layer tablet, and a multilayer tablet.

There is no particular restriction on the specific shape and size of each of the above-mentioned dosage forms, as long as the respective dosage forms are within the ranges of dosage forms publicly known in the art. As for the manufacturing method of the pharmaceutical composition of the present invention, it may be formulated according to the common procedure in the art.

### (Dosing Form and Dose)

Herein there is no particular restriction on the preferable dosing form of a pharmaceutical composition. Administration may be systemic or topical. The route of administration may be oral or parenteral. Specific examples of parenteral administration include intravenous, intra-arterial, blood transfusion, intraperitoneal, intraventricular, intrathecal, intraocular, intramuscular, subcutaneous (including implantable continuous subcutaneous administration), intradermal, intravesical, transvaginal, rectal, inhalation or intranasal, and tracheal/tracheal administration. When the target site of application of the present invention is the brain, intraventricular or intrathecal administration at the target site is suitable.

When a pharmaceutical composition is applied by administration or ingestion, the administered amount or ingested amount may be, for example, from 0.00001 mg/kg/day to 10000 mg/kg/day, or from 0.001 mg/kg/day to 100 mg/kg/day for the nucleic acid molecule or the double-stranded nucleic acid complex contained in the pharmaceutical composition. A pharmaceutical composition may be applied by single-dose administration or multiple dose administration. In the case of multiple dose administration, it may be administered daily or at appropriate time intervals (e.g., at intervals of 1 day, 2 days, 3 days, 1 week, 2 weeks, or 1 month), for example, for 2 to 20 times. A single dose of the nucleic acid molecule or double-stranded nucleic acid complex described above may be, for example, 0.001 mg/kg or more, 0.005 mg/kg or more, 0.01 mg/kg or more, 0.1 mg/kg or more, 0.25 mg/kg or more, 0.5 mg/kg or more, 1 mg/kg or more, 2.5 mg/kg or more, 0.5 mg/kg or more, 1.0 mg/kg or more, 2.0 mg/kg or more, 3.0 mg/kg or more, 4.0 mg/kg or more, 5 mg/kg or more, 10 mg/kg or more, 20 mg/kg or more, 30 mg/kg or more, 40 mg/kg or more, 50 mg/kg or more, 75 mg/kg or more, 100 mg/kg or more, 150 mg/kg or more, 200 mg/kg or more, 300 mg/kg or more, 400 mg/kg or more, or 500 mg/kg or more. For example, any amount in the range of from 0.001 mg/kg to 500 mg/kg (e.g., 0.001 mg/kg, 0.01 mg/kg, 0.1 mg/kg, 1 mg/kg, 5 mg/kg, 10 mg/kg, 50 mg/kg, 100 mg/kg, or 200 mg/kg) may be selected as appropriate.

The nucleic acid molecule or double-stranded nucleic acid complex of the present invention may be administered twice a week for total four times at a dose of from 0.01 to 10 mg/kg (e.g., about 6.25 mg/kg). Alternatively, the nucleic acid molecule or the double-stranded nucleic acid complex may be administered once or twice a week for total two to four times, for example at a frequency of twice a week for total two times, at a dose of from 0.05 to 30 mg/kg (e.g., about 25 mg/kg). By adopting such a dosing regimen (divided administration), the toxicity can be lowered (e.g., avoidance of platelet reduction) compared to a single-dose administration at a higher dose, and the stress to the subject can be reduced.

Even when the pharmaceutical composition is repeatedly administered, its inhibitory effect can be produced additively in a cell. In the case of repeated administration, the efficacy can be improved with certain administration intervals (e.g., half a day or longer).

According to the present invention, a nucleic acid molecule and/or a double-stranded nucleic acid complex targeting the transcriptional product of a mutant BEAN1 gene described herein and/or a nucleic acid molecule and/or a double-stranded nucleic acid complex targeting the transcriptional product of a mutant TK2 gene, for use in treatment of the spinocerebellar ataxia type 31 (SCA31), are also provided.

According to the present invention, a nucleic acid molecule and/or a double-stranded nucleic acid complex targeting the transcriptional product of a mutant ATXN10 gene described herein, for use in treatment of spinocerebellar ataxia type 10 (SCA10), are also provided.

### <Method>

In one aspect, the present invention relates to a method for producing an antisense effect on a transcriptional product, comprising administering to a subject a nucleic acid molecule, a double-stranded nucleic acid complex, or a pharmaceutical composition as described herein. The method may be a method for treating or preventing a disease of a subject.

### <Treatment Method>

In one aspect, the present invention relates to a treatment method comprising administering to a patient an effective amount of a nucleic acid molecule, a double-stranded nucleic acid complex, or a pharmaceutical composition as described herein. A disease targeted by the treatment method of the present aspect is spinocerebellar ataxia type 31 (SCA31) or spinocerebellar ataxia type 10 (SCA10). The method for treating spinocerebellar ataxia type 31 (SCA31) comprises administering to a patient, effective amounts of a nucleic acid molecule and/or a double-stranded nucleic acid complex targeting the transcriptional product of a mutant BEAN1 gene described herein and/or a nucleic acid molecule and/or a double-stranded nucleic acid complex targeting the transcriptional product of a mutant TK2 gene. The method for treating spinocerebellar ataxia type 10 (SCA10) comprises administering to a patient effective amounts of a nucleic acid molecule and/or a double-stranded nucleic acid complex targeting the transcriptional product of a mutant ATXN10 gene described herein.

### <Production Method>

Those skilled in the art can produce the nucleic acid molecule, double-stranded nucleic acid complex, or pharmaceutical composition of the present invention by appropriately selecting a publicly known method. Usually, without limitation, firstly each of the first nucleic acid strand and the second nucleic acid strand that constitute a nucleic acid molecule or a double-stranded nucleic acid complex is designed and prepared. For example, the nucleic acid molecule or the first nucleic acid strand is designed based on the information on the base sequence of the target transcriptional product (e.g., the base sequence of the target gene), and the second nucleic acid strand is designed as a complementary strand thereto. Then, based on the information on the designed base sequences, each nucleic acid strand may be synthesized by a phosphoroamidite method using a commercially available automatic nucleic acid synthesizer, such as that from GE Healthcare, Thermo Fisher Scientific, or Beckman Coulter. Thereafter the prepared oligonucleotides may be purified using a reverse-phase column, an ion-exchange column, or the like.

In the case of a double-stranded nucleic acid complex to which a functional moiety is bound, a first nucleic acid strand may be produced according to the above method. Meanwhile, with respect to a second nucleic acid strand to which a functional moiety is bound, it may be produced by performing the above-described synthesis and purification using a nucleic acid species to which a functional moiety has been bound in advance. For example, a second nucleic acid strand may be produced by performing the above-described synthesis and purification using a nucleic acid species to which a functional moiety has been bound in advance. Alternatively, a functional moiety may be joined by a publicly known method to a second nucleic acid strand produced by performing the above-described synthesis and purification. After preparation of each nucleic acid strand, a double-stranded nucleic acid complex to which the functional moiety of interest is bound can be produced by performing annealing described below for the first nucleic acid strand and the second nucleic acid strand.

The method for linking a functional moiety to a nucleic acid is well known in the art. The nucleic acids produced by this method are mixed in an appropriate buffer solution to be denatured at about 90° C. to 98° C. for several minutes (e.g., 5 min), and then the nucleic acids are annealed in a range of about 30° C. to 70° C. for about 1 to 8 hours to yield a double-stranded nucleic acid complex of the present invention. Further, a nucleic acid strand can be obtained by ordering from various manufacturers (e.g., GeneDesign Inc.) by specifying the base sequence and the modification site and type. The above-described annealing step can be performed by allowing the nucleic acids to stand at room temperature (about 10° C. to 35° C.) for about 5 to 60 minutes. It is possible that the first nucleic acid strand and the second nucleic acid strand may be independently dissolved in a buffer solution (e.g., phosphate-buffered saline) or water at about 70° C. to 98° C., and the obtained two solutions are mixed, and the mixed liquid is kept at about 70° C. to 98° C. for several minutes (e.g., 5 min), and then the same is maintained at about 30° C. to 70° C. (or about 30° C. to 50° C.) for about 1 to 8 hours to prepare a double-stranded nucleic acid complex of some embodiments of the present invention. It is also possible that the first nucleic acid strand and the second nucleic acid strand are independently dissolved in a buffer solution (e.g., phosphate-buffered saline) or water at room temperature (about 10° C. to 35° C.). The annealing conditions (time and temperature) in preparing a double-stranded nucleic acid complex are not limited to the above conditions. In addition, the conditions suitable for promoting annealing of nucleic acid strands are well known in the art.

In one embodiment, use of a nucleic acid molecule and/or a double-stranded nucleic acid complex targeting the transcriptional product of a mutant BEAN1 gene described herein and/or a nucleic acid molecule and/or a double-stranded nucleic acid complex targeting the transcriptional product of a mutant TK2 gene, for use in treatment of spinocerebellar ataxia type 31 (SCA31), is provided.

In another embodiment, use of a nucleic acid molecule and/or a double-stranded nucleic acid complex targeting the transcriptional product of a mutant ATXN10 gene described herein, for use in treatment of spinocerebellar ataxia type 10 (SCA10), is provided.

### EXAMPLES

The present invention will be specifically described below with reference to Examples. These examples are intended to explain the present invention, but are not intended to limit the scope of the present invention.

### Example 1: Synthesis of ASO (LNA/DNA Gapmer) Targeting SCA31 Mutant Repeat (TK2)

GeneDesign Inc. (Osaka, Japan) was requested to synthesize antisense oligonucleotides (ASOs) targeting an SCA31 abnormal repeat region in the transcriptional product of a mutant TK2 gene (hereinafter referred to as “SCA31 mutant repeat (TK2)”).

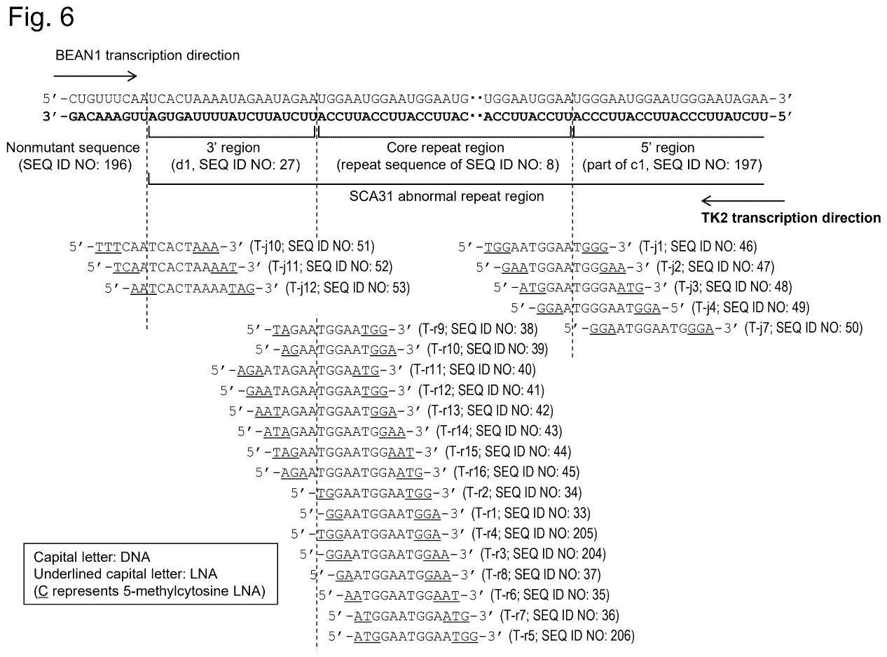

The synthesized ASOs are shown in Table 1 and FIG. 6. All of the ASOs synthesized in the present example are LNA/DNA gapmers.

TABLE 1

| | Compound | Sequence | SEQ ID |
|---|---|---|---|
| Example | name | (5′-3′) | NO: |
| 1-1 | T-r1 | GGAATGGAATGGA | 33 |
| 1-2 | T-r2 | TGGAATGGAATGG | 34 |
| 1-3 | T-r3 | GGAATGGAATGGAA | 204 |
| 1-4 | T-r4 | TGGAATGGAATGGA | 205 |
| 1-5 | T-r5 | ATGGAATGGAATGG | 206 |
| 1-6 | T-r6 | AATGGAATGGAAT | 35 |
| 1-7 | T-r7 | ATGGAATGGAATG | 36 |
| 1-8 | T-r8 | GAATGGAATGGAA | 37 |
| 1-9 | T-r9 | TAGAATGGAATGG | 38 |
| 1-10 | T-r10 | AGAATGGAATGGA | 39 |
| 1-11 | T-r11 | AGAATAGAATGGAATG | 40 |
| 1-12 | T-r12 | GAATAGAATGGAATGG | 41 |
| 1-13 | T-r13 | AATAGAATGGAATGGA | 42 |
| 1-14 | T-r14 | ATAGAATGGAATGGAA | 43 |
| 1-15 | T-r15 | TAGAATGGAATGGAAT | 44 |
| 1-16 | T-r16 | AGAATGGAATGGAATG | 45 |
| 1-17 | T-j1 | TGGAATGGAATGGG | 46 |
| 1-18 | T-j2 | GAATGGAATGGGAA | 47 |
| 1-19 | T-j3 | ATGGAATGGGAATG | 48 |
| 1-20 | T-j4 | GGAATGGGAATGGA | 49 |
| 1-21 | T-j7 | GGAATGGAATGGGA | 50 |
| 1-22 | T-j10 | TTTCAATCACTAAA | 51 |
| 1-23 | T-j11 | TCAATCACTAAAAT | 52 |
| 1-24 | T-j12 | AATCACTAAAATAG | 53 |

Capital letter: DNA; underlined capital letter: LNA. All the internucleoside linkages are phosphorothioate linkages. The nucleobase C in LNA is 5-methylcytosine.

### Example 2: Investigation of Effect of Reducing Gene Expression by LNA/DNA Gapmer Synthesized in Example 1

### (Purpose)

The effect of reducing gene expression of SCA31 mutant repeats (TK2) by the ASOs (LNA/DNA gapmers) synthesized in Example 1 is investigated.

### (Method and Results)

(1) qRT-PCR

Transient expression of SCA31 mutant repeats (TK2) is conducted, the ASOs produced in Example 1 are introduced into HeLa cells, and the effect of reducing gene expression of the SCA31 mutant repeats (TK2) is investigated.

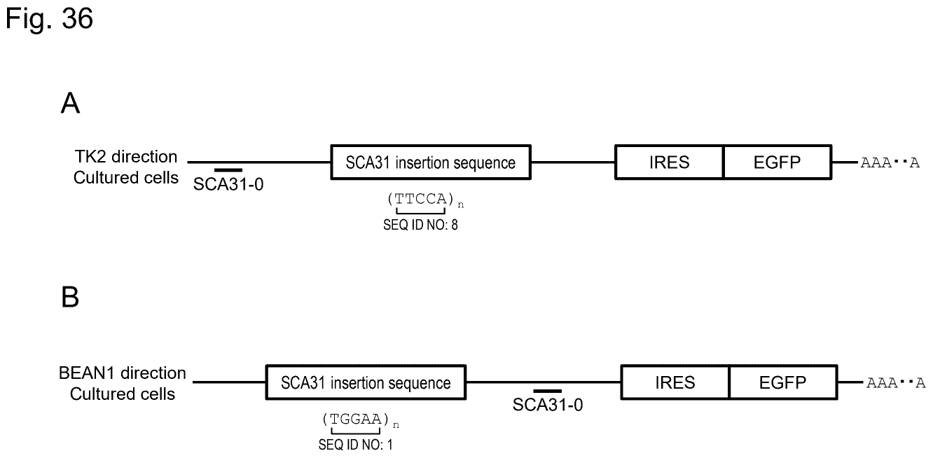

For the transient expression of the SCA31 mutant repeats (TK2), the sequences of the SCA31 mutant repeats (TK2) were positioned downstream of a chicken R-actin promoter, and a pkSCX-IRES-EGFP vector having IRES-EGFP was used just after them (FIG. 36A). In addition, 0.8 μg of pkSCX-IRES-EGFP vector was diluted with 50 μL of Opti-MEM. For each ASO, a stock solution was produced in 10 μM in PBS.

In Examples herein, unless otherwise specified, PBS (shown as “Control” in the drawings) was used as a negative control, and PBS of which the amount was equal to that of ASO to be introduced was added when a gene was introduced.

Moreover, ASO consisting of a base sequence which was not related to a target sequence was used as a further control (shown as “Unrelated” in the drawings), and any sequence shown in the following Table 2 was used.

TABLE 2

| Compound name | | Sequence (5′-3′) | SEQ ID NO: |
|---|---|---|---|
| Unrelated | ApoB | GCATTGGTATTCA | 54 |
| | ApoB shuffle | CGTAGTTGTAATC | 55 |
| | mMALAT1 | CTAGTTCACTGAATGC | 56 |
| | SNCA-R1 | CCAACATTTGTCA | 57 |

Capital letter: DNA; underlined capital letter: LNA. All the internucleoside linkages are phosphorothioate linkages. A nucleobase C in LNA is 5-methylcytosine.

The ASOs (0.1 to 2 nM) were cotransduced together with a pkSCX-IRES-EGFP vector (FIG. 36A) comprising an SCA31 insertion sequence in the TK2 direction into 1.2×10⁵/μL of HeLa cells using 50 μL of Opti-MEM comprising 2 μL of lipofectamin 2000. RNA was collected from the HeLa cells by extraction with ISOGEN and an isopropanol sedimentation method 24 hours after the transduction. Then, 250 ng of the collected RNA was converted into cDNA using PrimeScript RT Reagent (Takara Bio). By using Roche Light Cycler 48011 using a probe primer mixture: SCA31-0 (TaqMan) consisting of SCA31-0_primer FW (SEQ ID NO: 18, 5′-TGGCTGCACATAGCTTTATCTCTT-3′) and SCA31-0_primer RV (SEQ ID NO: 20, 5′-AAGCCCAATCTGGAAGCAAA-3′) using this stock solution as a template, qRT-PCR was performed. The position detected by qRT-PCR is shown as “SCA31-0” in FIG. 36A. In reference, each result was analyzed by ΔΔCt method using ACTB (Hs01060665-g1, ABI), and the significance thereof was analyzed by t-test.

To confirm transduction efficiency, ds-Red of which the amount was 1/40 of the amount of the pkScx-IRES-EGFP vector was cotransduced, and no difference in fluorescence intensity was confirmed after 24 hours.

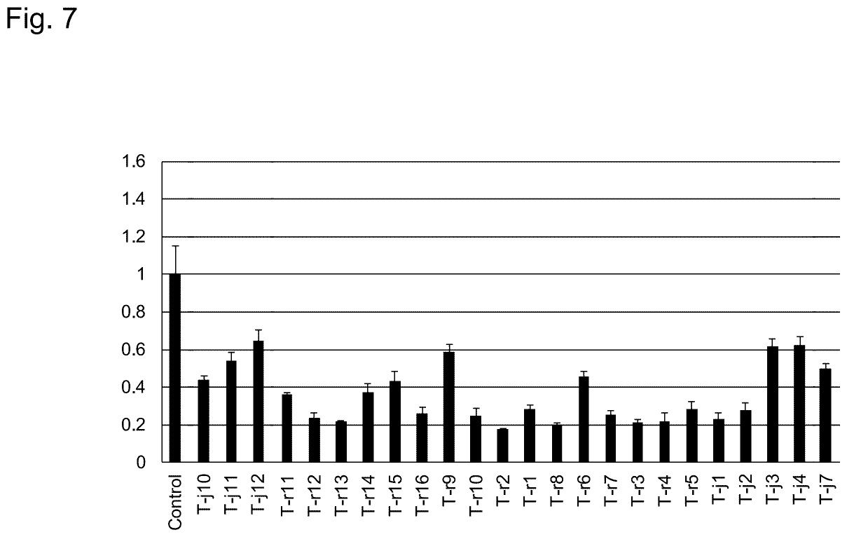

The results of qRT-PCR are shown in FIG. 7. The strong effects of reducing expression of the SCA31 mutant repeats (TK2) were obtained particularly by T-j1, T-j2, T-r2, T-r1, T-r3, T-r4, T-r5, T-r8, T-r7, T-r12, T-r13, T-r16, and T-r10. The results revealed that the strong effect of reducing expression tends to be obtained when the target sequence of ASO comprises the boundary between the 5′ region and the core repeat region, when the target sequence of ASO comprises the boundary between the core repeat region and the 3′ region, or when the target sequence of ASO is positioned in the interior side of the core repeat region.

### (2) FISH

T-r1 (SEQ ID NO: 33) was selected from the ASOs used in (1) as described above, and the effect of reducing gene expression of the SCA31 mutant repeats (TK2) was investigated by FISH. Likewise, the other sequences were investigated.

ASO (T-r1) was introduced together with the pkSCX-IRES-EGFP vector comprising an SCA31 insertion sequence in the TK2 direction into HeLa cells by a method similar to that in (1) as described above. The cells were washed with 1×PBS for 5 minutes 24 hours after the gene introduction, and fixed in advance in 4% PFA for 1 hour. The cells were washed with 1×PBS for 5 minutes, then reacted with 0.2 N HCl for 20 minutes to break the cell walls, and subjected to permeabilization treatment in 0.1% TritonX-100 for 10 minutes. The cells were washed with 1×PBS for 5 minutes, fixed later in 4% PFA for 5 minutes, and then acetylated in an acetic anhydride triethanolamine solution for 20 minutes. The cells were washed twice in 4×SSC for 5 minutes, and subjected to prehybridization for 30 minutes, followed by hybridizing an LNA-(TGGAA)₅ probe labelled with DIG (60° C., 2 hours). At 60° C., the cells were washed with 4×SSC for 5 minutes, three times with 2×SSC/Formamide for 20 minutes, and three times with 0.1×SSC for 40 minutes. The cells were blocked for 30 minutes, and allowed to react with an anti-DIG-AP antibody of 1/2000 at 4° C. overnight. The cells were washed four times with TBS-t for 15 minutes, then allowed to react with a detection solution (0.1 M Tris-HCl, 10 mM MgCl₂, 0.1 M NaCl) for 10 minutes, and then colored with HNPP/FastRed for 30 minutes. Finally, the cells were nuclear-stained with Hoechst for 10 minutes, and embedded in VECTASHEILD.

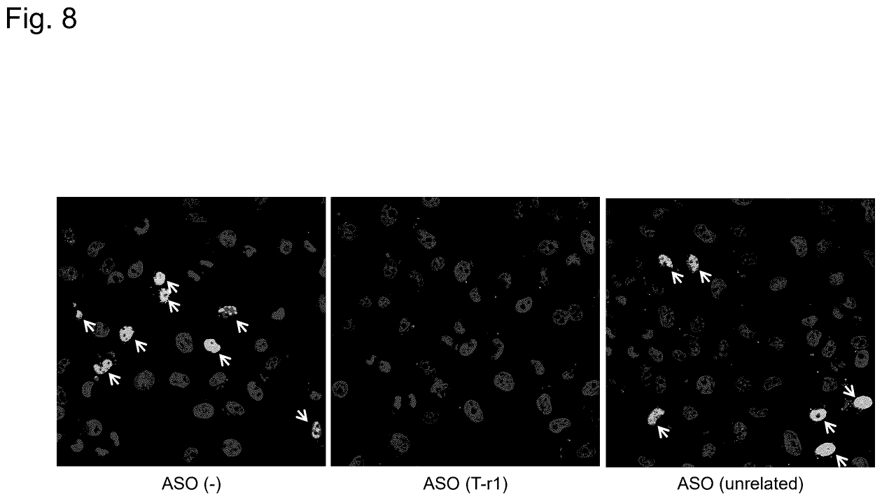

The results of FISH are shown in FIG. 8. In FIG. 8, the cells in which the expression of the SCA31 mutant repeats (TK2) was detected are indicated by the arrows. It was shown that the expression of the SCA31 mutant repeats (TK2) was suppressed by ASO (T-r1).

### Example 3: Synthesis of ENA/DNA Gapmer Targeting SCA31 Mutant Repeats (TK2)

A method for synthesizing ASO targeting SCA31 mutant repeats (TK2) in the present example is explained by taking T-re10:

(SEQ ID NO: 59)
HO-T^{e2s}-G^{e2s}-G^{s}-A^{s}-A^{s}-T^{s}-G^{s}-G^{s}-A^{s}-A^{s}-T^{e2s}-G^{e2s}-G^{2t}-H

(the meaning of each symbol in the formula is as described above)
as an example.

The synthesis was performed by using a phosphoroamidite method (Nucleic Acids Research, 12, 4539 (1984) using a nucleic acid automatic synthesizer (MerMade 192X manufactured by BioAutomation). Activator solution-3 (0.25 mol/L of 5-benzylthio-1H-tetrazole/acetonitrile solution, manufactured by Wako Pure Chemical Industries, Ltd., product No. 013-20011), CAP A for AKTA (1-methylimidazole/acetonitrile solution, manufactured by Sigma-Aldrich, product No. L040050), Cap B1 for AKTA (acetic anhydride/acetonitrile solution, manufactured by Sigma-Aldrich, product No. L050050), Cap B2 for AKTA (pyridine/acetonitrile solution, manufactured by Sigma-Aldrich, product No. L050150), and DCA Deblock (dichloroacetic acid/toluene solution, manufactured by Sigma-Aldrich, product No. L023050) were used as reagents. A solution obtained by dissolving phenylacetyl disulfide (manufactured by CARBOSYNTH, product No. FP07495) in acetonitrile (dehydrated, manufactured by KANTO CHEMICAL CO., INC., product No. 01837-05) and pyridine (dehydrated, manufactured by KANTO CHEMICAL CO., INC., product No. 11339-05) 1:1 (v/v) solution to be 0.2 M was used as a thiation reagent for forming a phosphorothioate linkage. Phosphoroamidites of DNA (adenosine body product No. ANP-5551, guanosine body product No. ANP-5553, and thymidine body product No. ANP-5554) and phosphoroamidites of 2′-OMe nucleoside (adenosine body product No. ANP-5751, cytidine body product No. ANP-5752, guanosine body product No. ANP-5753, and uridine body product No. ANP-5754), manufactured by ChemGenes, were used as amidite reagents. A compound in 6f in a cited literature (Organic Process Research & Development, 2000, 4, 175-181) was used as 5-methyldeoxycytidine phosphoroamidite. Compounds in Example 14 (5′-O-dimethoxytrityl-2′-O,4′-C-ethylene-6-N-benzoyladenosine-3′-O-(2-cyanoethyl N,N-diisopropyl)phosphoroamidite), Example 27 (5′-O-dimethoxytrityl-2′-O,4′-C-ethylene-2-N-isobutyrylguanosine-3′-O-(2-cyanoethyl N,N-diisopropyl)phosphoroamidite), Example 22 (5′-O-dimethoxytrityl-2′-0,4′-C-ethylene-4-N-benzoyl-5-methylcytidine-3′-O-(2-cyanoethyl N,N-diisopropyl)phosphoroamidite), Example 9 (5′-O-dimethoxytrityl-2′-O,4′-C-ethylene-5-methyluridine-3′-O-(2-cyanoethyl N,N- diisopropyl)phosphoroamidite) in Japanese Patent Laid-Open No. 2000-297097 were used as non-natural phosphoroamidites. As a solid phase carrier, 0.2 μmol of Glen Unysupport FC 96-well format (manufactured by GlenResearch) was used to synthesize T-re10 (SEQ ID NO: 59). However, time required for condensation of the amidite bodies was set at about 9 minutes.

An oligomer was cleaved down from a support by treating a protected oligonucleotide analog having a sequence of interest with 600 μL of concentrated aqueous ammonia, and a protecting cyanoethyl group on a phosphorus atom and a protecting group on a nucleobase were removed. A mixed solution of an oligomer was mixed with 300 μL of Clarity QSP DNA Loading Buffer (manufactured by Phenomenex), and the mixture was charged on a Clarity SPE 96-well plate (manufactured by Phenomenex). Addition of 1 mL of Clarity QSP DNA Loading Buffer:water=1:1 solution, 3 mL of water, 3 mL of 3% aqueous dichloroacetic acid (DCA) solution, and 6 mL of water in the order mentioned above was performed, followed by collecting components extracted with 20 mM aqueous Tris solution:acetonitrile=9:1 solution. The solvent was distilled off, and a compound of interest was obtained. This compound was analyzed by reverse phase HPLC (column (Phenomenex, Clarity 2.6 μm Oligo-MS 100A (2.1×50 mm)), solution A: 100 mM hexafluoroisopropanol (HFIP), 8 mM aqueous triethylamine solution, solution B: methanol, B %: 10%→25% (4 min, linear gradient); 60° C.; 0.5 mL/min; 260 nm), and thus eluted at 2.12 minutes. The compound was identified by negative ion ESI mass spectrometry.

The synthesized ASOs are shown in Table 3. All of the ASOs synthesized in the present example are ENA/DNA gapmers.

TABLE 3

| | Compound | | Molecular | SEQ ID |
|---|---|---|---|---|
| Example | name | Sequence (5′-3′) | weight | NO: |
| 3-1 | T-re01 | GGAATGGAATGGA | 4487.52 | 58 |
| 3-2 | T-re10 | TGGAATGGAATGG | 4478.52 | 59 |
| 3-3 | T-re11 | GAATGGAATGGAA | 4471.54 | 60 |
| 3-4 | T-re13 | ATGGAATGGAATG | 4462.55 | 61 |
| 3-5 | T-re18 | AGAATGGAATGGA | 4471.48 | 62 |
| 3-6 | T-re19 | AGAATGGAATGGAATG | 5507.60 | 63 |

Capital letter: DNA; underlined capital letter: ENA. All the internucleoside linkages are phosphorothioate linkages. Each molecular weight shows the value actually measured by negative ion ESI mass spectrometry.

### Example 4: Investigation of Effect of Reducing Gene Expression by ASOs (ENA/DNA Gapmers) Synthesized in Example 3

### (Purpose)

The effect of reducing gene expression of SCA31 mutant repeats (TK2) by the ASOs (ENA/DNA gapmers) synthesized in Example 3 is investigated.

### (Method and Results)

ASOs were introduced together with a pkSCX-IRES-EGFP vector comprising an SCA31 insertion sequence in the TK2 direction into HeLa cells by a method similar to that in Example 2, and the expression levels of SCA31 mutant repeats (TK2) were quantified by qRT-PCR. As the ASOs, the LNA/DNA gapmers synthesized in Example 1 and the ENA/DNA gapmers synthesized in Example 3 were used in 0.1 nM, 0.5 nM, or 2 nM.

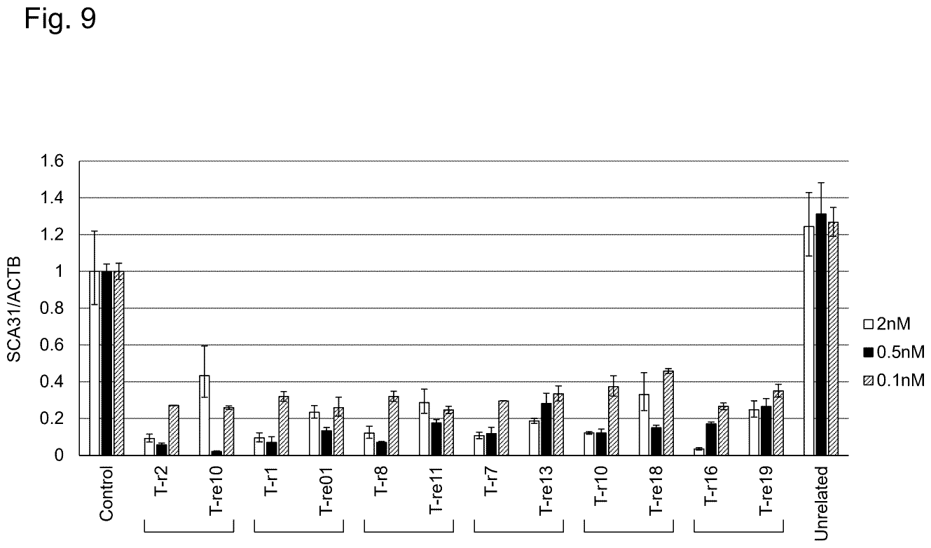

The results of the quantification of the expression levels of the SCA31 mutant repeats (TK2) are shown in FIG. 9. The results revealed that expression-reducing effects equivalent to those in the LNA/DNA gapmers were obtained by the ENA/DNA gapmers.

### Example 5: ASO Targeting SCA31 Mutant Repeat (TK2): Gapmer Having ENA-2′-OMe RNA-ENA Wing Region

### (Purpose)

An ENA/DNA gapmer having an ENA-2′-OMe RNA-ENA structure in a wing region is synthesized, and the effect of reducing gene expression of SCA31 mutant repeats (TK2) is investigated.

### (Method and Results)

ASOs were synthesized by a method similar to that in Example 3. The synthesized ASOs are shown in Table 4. All of the ASOs synthesized in the present example are ENA/DNA gapmers having ENA-2′-OMe RNA-ENA wing regions.

TABLE 4

| | Compound | | Molecular | SEQ ID |
|---|---|---|---|---|
| Example | name | Sequence (5′-3′) | weight | NO: |
| 5-1 | T-re01a | GgAATGGAATGgA | 4505.51 | 64 |
| 5-2 | T-re10a | TgGAATGGAATgG | 4496.47 | 65 |
| 5-3 | T-re11a | GaATGGAATGGaA | 4489.50 | 66 |
| 5-4 | T-re13a | AuGGAATGGAAuG | 4452.48 | 67 |
| 5-5 | T-re18a | AgAATGGAATGgA | 4489.53 | 68 |
| 5-6 | T-re19a | AgAATGGAATGGAAuG | 5469.58 | 69 |

Capital letter: DNA; small letter: 2′-OMe RNA; underlined capital letter: ENA. All the internucleoside linkages are phosphorothioate linkages. Each molecular weight shows a value actually measured by negative ion ESI mass spectrometry.

ASOs were introduced together with a pkSCX-IRES-EGFP vector comprising an SCA31 insertion sequence in the TK2 direction into HeLa cells by a method similar to that in Example 2, and the expression levels of SCA31 mutant repeats (TK2) were quantified by qRT-PCR. The ASOs were used in 0.1 nM, 0.5 nM, 1 nM, or 2 nM.

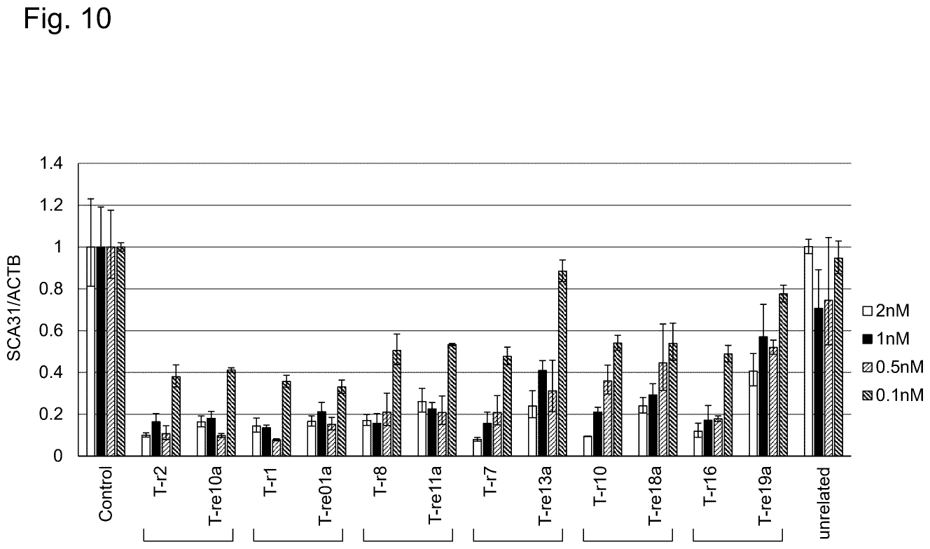

The results of the quantification of the expression levels of the SCA31 mutant repeats (TK2) are shown in FIG. 10. The results revealed that expression-reducing effects equivalent to those by the LNA/DNA gapmers were obtained in the ENA/DNA gapmers having ENA-2′-OMe RNA-ENA wing regions.

### Example 6: ASO Targeting SCA31 Mutant Repeats (TK2): Surrounding Sequence of T-Re10a

### (Purpose)

ASO consisting of the surrounding sequence of T-re10a exhibiting a strong effect of reducing expression of the SCA31 mutant repeats (TK2) in Example 5 is synthesized, and the effect is investigated.

### (Method and Results)

ASOs were synthesized by a method similar to that in Example 3. The synthesized ASOs are shown in Table 5.

TABLE 5

| | Compound | | Molecular | SEQ ID |
|---|---|---|---|---|
| Example | name | Sequence (5′-3′) | weight | NO: |
| 6-1 | T-re10e | TgGAATGGAATgg | 4484.51 | 70 |
| 6-2 | T-re10f | TggAATGGAATgG | 4484.51 | 71 |
| 6-3 | T-re10g | TggAATGGAATgg | 4472.51 | 72 |
| 6-4 | T-re10h | uGgAATGGAAuGg | 4444.49 | 73 |
| 6-5 | T-re10i | aTgGAATGGAATgG | 4855.54 | 74 |
| 6-6 | T-re10j | TgGAATGGAATgGa | 4855.55 | 75 |
| 6-7 | T-re10k | aTgGAATGGAATgGa | 5214.60 | 76 |
| 6-8 | T-re101 | ATgGAATGGAATgG | 4867.55 | 77 |
| 6-9 | T-re10m | TgGAATGGAATgGA | 4867.56 | 78 |
| 6-10 | T-re10n | ATgGAATGGAATgGA | 5238.60 | 79 |
| 6-11 | T-re100 | ATggAATGGAATggA | 5214.61 | 80 |
| 6-12 | T-re10p | auGGAATGGAATGga | 5200.59 | 81 |

Capital letter: DNA; small letter: 2′-OMe RNA; underlined capital letter: ENA. All the internucleoside linkages are phosphorothioate linkages. Each molecular weight shows a value actually measured by negative ion ESI mass spectrometry.

ASOs (0.1 nM) were introduced together with a pkSCX-IRES-EGFP vector comprising an SCA31 insertion sequence in the TK2 direction into HeLa cells by a method similar to that in Example 2, and the expression levels of SCA31 mutant repeats (TK2) were quantified by qRT-PCR.

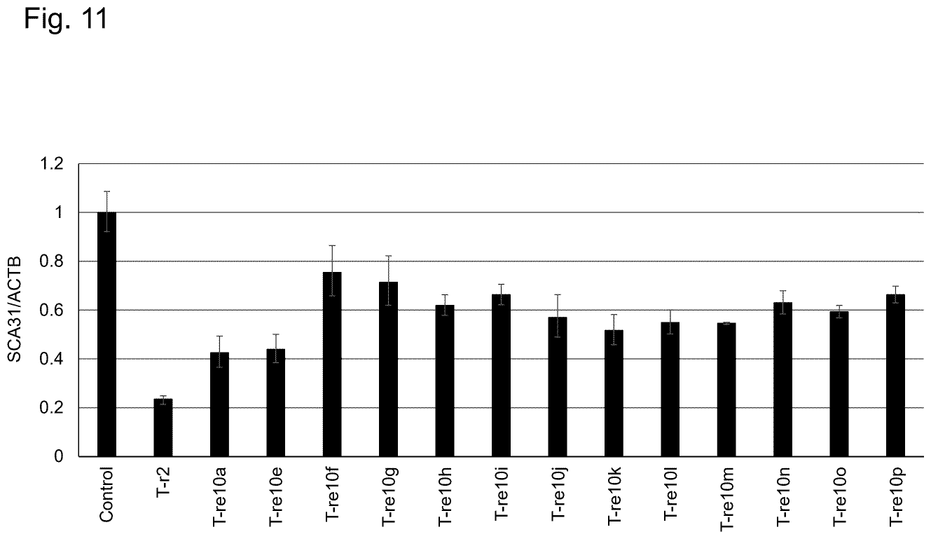

The results of the quantification of the expression levels of the SCA31 mutant repeats (TK2) are shown in FIG. 11. Among the ASOs shown in Table 5, T-re10e exhibited an effect of reducing expression equivalent to that of T-re10a. T-re10f to T-re10p also exhibited an effect of reducing expression. Among these compounds, T-re10a and T-re10e were revealed to have the strongest effect of reducing expression.

### Example 7: ENA/DNA Gapmer Targeting Interior Part of the Core Repeat Region of SCA31 Mutant Repeats (TK2)

### (Purpose)

An ENA/DNA gapmer targeting the interior part of the core repeat region of SCA31 mutant repeats (TK2) and having an ENA-2′-OMe RNA-ENA structure in a wing region is synthesized, and the effect of reducing gene expression of the SCA31 mutant repeats (TK2) is investigated.

### (Method and Results)

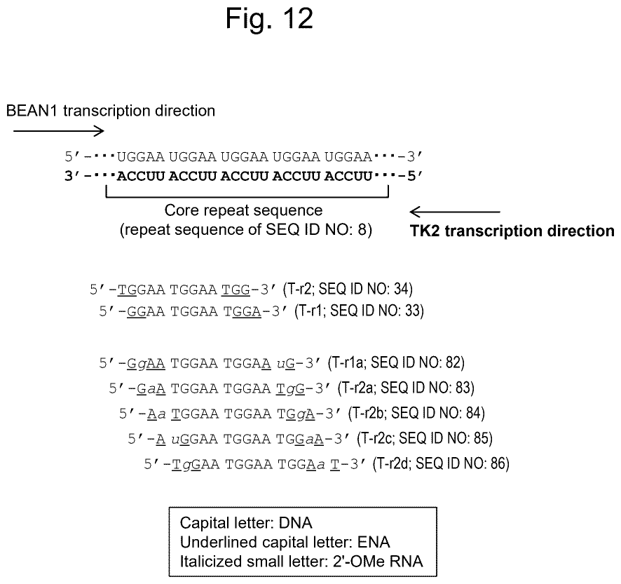

ASOs were synthesized by a method similar to that in Example 3. The synthesized ASOs are shown in Table 6. Moreover, the position of each ASO in the SCA31 mutant repeats (TK2) is shown in FIG. 12.

TABLE 6

| | Compound | | SEQ ID |
|---|---|---|---|
| Example | name | Sequence (5′-3′) | NO: |
| 7-1 | T-r1a | GgAATGGAATGGAAuG | 82 |
| 7-2 | T-r2a | GaATGGAATGGAATgG | 83 |
| 7-3 | T-r2b | AaTGGAATGGAATGgA | 84 |
| 7-4 | T-r2c | AuGGAATGGAATGGaA | 85 |
| 7-5 | T-r2d | TgGAATGGAATGGAaT | 86 |

Capital letter: DNA; small letter: 2′-OMe RNA; underlined capital letter: ENA. All the internucleoside linkages are phosphorothioate linkages.

ASOs (0.1 nM) were introduced together with a pkSCX-IRES-EGFP vector comprising an SCA31 insertion sequence in the TK2 direction into HeLa cells by a method similar to that in Example 2, and the expression levels of SCA31 mutant repeats (TK2) were quantified by qRT-PCR.

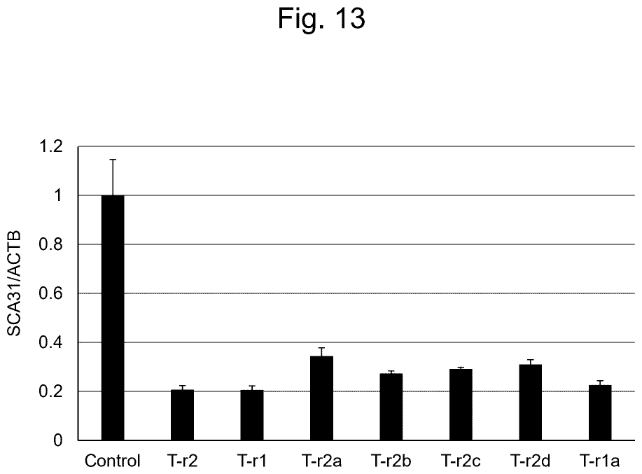

The results of the quantification of the expression levels of the SCA31 mutant repeats (TK2) are shown in FIG. 13. All of T-r1a, T-r2a, T-r2b, T-r2c, and T-r2d exhibited strong effects of reducing gene expression. The effect of reducing expression by T-r1a was found to be particularly strong.

### Example 8: ASO Targeting Interior Part of Core Repeat Region of SCA31 Mutant Repeat (TK2): MOE/DNA Gapmer

### (Purpose)

An ASO (MOE/DNA gapmer) targeting the interior part of the core repeat region of SCA31 mutant repeats (TK2) is synthesized, and the effect of reducing gene expression of the SCA31 mutant repeats (TK2) is investigated.

### (Method and Results)

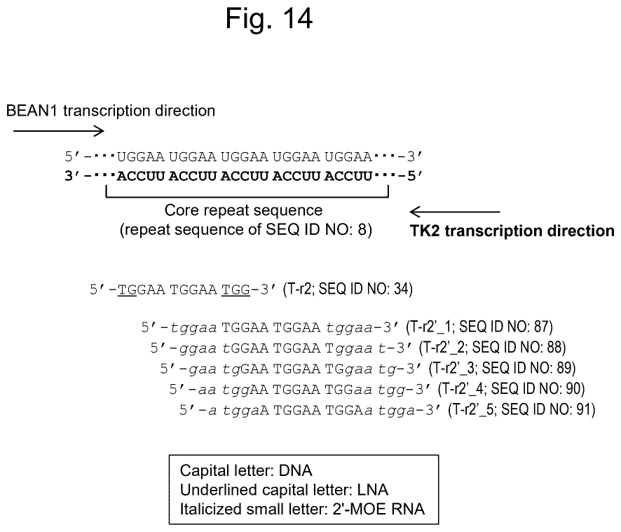

ASOs were synthesized by a method similar to that in Example 3. The synthesized ASOs are shown in Table 7. Moreover, the position of each ASO in the SCA31 mutant repeats (TK2) is shown in FIG. 14.

TABLE 7

| | Compound | | SEQ ID |
|---|---|---|---|
| Example | name | Sequence (5′-3′) | NO: |
| 8-1 | T-r2′_1 | tggaaTGGAATGGAAtggaa | 87 |
| 8-2 | T-r2′ 2 | ggaatGGAATGGAATggaat | 88 |
| 8-3 | T-+2′ 3 | gaatgGAATGGAATGgaatg | 89 |
| 8-4 | T-12′ 4 | aatggAATGGAATGGaatgg | 90 |
| 8-5 | T-r2′ 5 | atggaATGGAATGGAatgga | 91 |

Capital letter: DNA; underlined small letter: 2′-OMe RNA. All the internucleoside linkages are phosphorothioate linkages.

ASOs (0.5 nM or 2 nM) were introduced together with a pkSCX-IRES-EGFP vector comprising an SCA31 insertion sequence in the TK2 direction into HeLa cells by a method similar to that in Example 2, and the expression levels of SCA31 mutant repeats (TK2) were quantified by qRT-PCR.

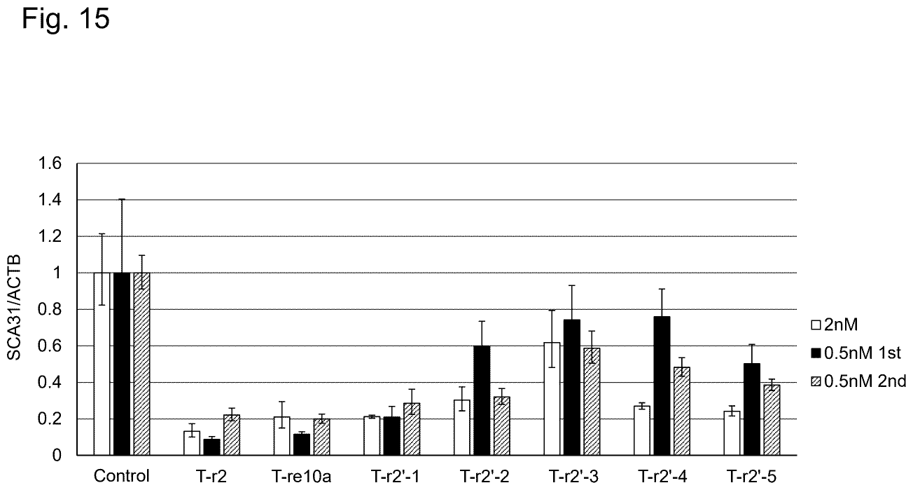

The results of the quantification of the expression levels of the SCA31 mutant repeats (TK2) are shown in FIG. 15. All of the ASOs shown in Table 7 exhibited an effect of reducing expression, and T-r2′-1 particularly exhibited a strong effect of reducing expression.

### Example 9: ASO Targeting SCA31 Mutant Repeats (TK2): MOE/ENA/DNA Gapmer

### (Purpose)

MOE/ENA/DNA gapmers in which the positions and numbers of MOEs and ENAs in a wing region are modified are synthesized, and the effect of reducing gene expression of SCA31 mutant repeats (TK2) is investigated.

### (Method and Results)

ASOs were synthesized by a method similar to that in Example 3. The synthesized ASOs are shown in Table 8.

TABLE 8

| | Compound | | Molecular | SEQ ID |
|---|---|---|---|---|
| Example | name | Sequence (5′-3′) | weight | NO: |
| 9-1 | T-r2′#1 | TggaaTGGAATGGAAtggaA | 7270.97 | 92 |
| 9-2 | T-r2′#2 | TggaaTGGAATGGAAtggAa | 7270.99 | 93 |
| 9-3 | T-r2′#3 | TggaaTGGAATGGAATggaa | 7270.98 | 94 |
| 9-4 | T-r2′#4 | tggAaTGGAATGGAAtggaA | 7270.96 | 95 |
| 9-5 | T-r2′#5 | tggAaTGGAATGGAAtggAa | 7270.96 | 96 |
| 9-6 | T-r2′#6 | tggAaTGGAATGGAATggaa | 7270.96 | 97 |
| 9-7 | T-r2′#7 | tggaATGGAATGGAAtggAa | 7270.96 | 98 |
| 9-8 | T-r2′#8 | tggaATGGAATGGAAtggaA | 7270.95 | 99 |
| 9-9 | T-r2′#9 | tggaATGGAATGGAATggaa | 7270.96 | 100 |
| 9-10 | T-r2′#10 | TggAaTGGAATGGAATggaa | 7270.96 | 101 |
| 9-11 | T-r2′#11 | TggAaTGGAATGGAAtggAa | 7238.99 | 102 |
| 9-12 | T-r2′#12 | TggAaTGGAATGGAAtggaA | 7238.95 | 103 |
| 9-13 | T-r2′#13 | TggaATGGAATGGAATggaa | 7238.95 | 104 |
| 9-14 | T-r2′#14 | TggaATGGAATGGAAtggAa | 7238.92 | 105 |
| 9-15 | T-r2′#15 | TggaATGGAATGGAAtggaA | 7238.98 | 106 |
| 9-16 | T-r2′#16 | TggaaTGGAATGGAATggAa | 7238.97 | 107 |
| 9-17 | T-r2′#17 | TggaaTGGAATGGAATggaA | 7238.97 | 108 |
| 9-18 | T-r2′#18 | tggAaTGGAATGGAATggAa | 7238.99 | 109 |
| 9-19 | T-r2′#19 | tggAaTGGAATGGAATggaA | 7238.90 | 110 |
| 9-20 | T-r2′#20 | tggaATGGAATGGAATggAa | 7238.92 | 111 |
| 9-21 | T-r2′#21 | tggaATGGAATGGAATggaA | 7238.97 | 112 |
| 9-22 | T-r2′#22 | TggAaTGGAATGGAATggAa | 7238.96 | 113 |
| 9-23 | T-r2′#23 | TggAaTGGAATGGAATggaA | 7206.90 | 114 |
| 9-24 | T-r2′#24 | TggaATGGAATGGAATggAa | 7206.90 | 115 |
| 9-25 | T-r2′#25 | TggaATGGAATGGAATggaA | 7206.90 | 116 |

Capital letter: DNA; underlined capital letter: ENA; underlined small letter: 2′-OMe RNA. All the internucleoside linkages are phosphorothioate linkages. Each molecular weight shows a value actually measured by negative ion ESI mass spectrometry.

ASOs (0.5 nM) were introduced together with a pkSCX-IRES-EGFP vector comprising an SCA31 insertion sequence in the TK2 direction into HeLa cells by a method similar to that in Example 2, and the expression levels of SCA31 mutant repeats (TK2) were quantified by qRT-PCR.

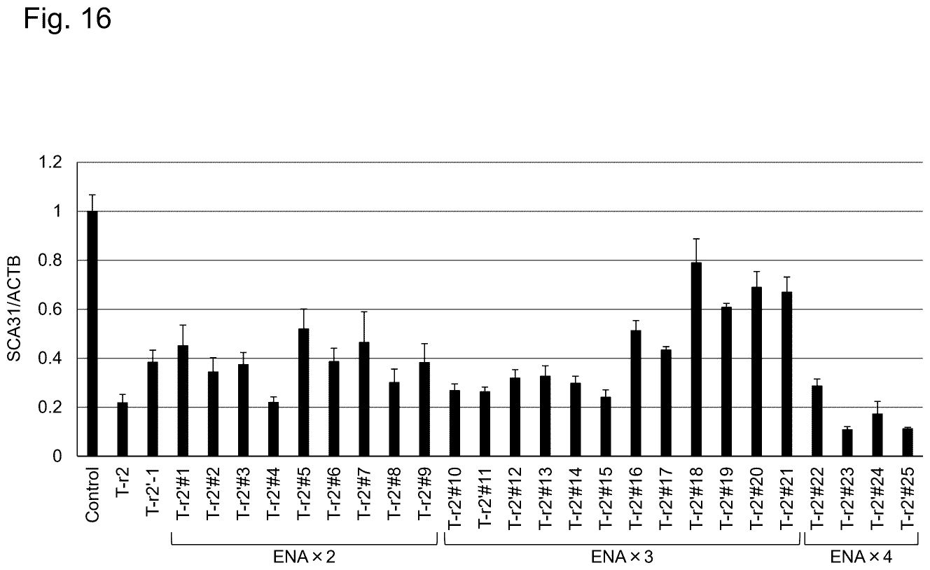

The results of the quantification of the expression levels of the SCA31 mutant repeats (TK2) are shown in FIG. 16. All of the ASOs shown in Table 8 exhibited a strong effect of reducing expression, and among them, T-r2′#4, T-r2′#15, T-r2′#23, T-r2′#24, and T-r2′#25 exhibited strong effects of reducing expression. T-r2′#23 and T-r2′#25 in which each of a 5′-wing region and a 3′-wing region comprises two ENAs exhibited particularly strong effects of reducing expression.

### Example 10: ASO Targeting SCA31 Mutant Repeats (TK2): MOE/ENA/DNA Gapmer

### (Purpose)

MOE/ENA/DNA gapmers in which the positions and numbers of MOEs and ENAs in a wing region are modified are synthesized, and the effect of reducing gene expression of SCA31 mutant repeats (TK2) is investigated.

### (Method and Results)

ASOs were synthesized by a method similar to that in Example 3. The synthesized ASOs are shown in Table 9.

TABLE 9

| | Compound | | SEQ ID |
|---|---|---|---|
| Example | name | Sequence (5′-3′) | NO: |
| 10-1 | T-r2′#26 | tggAATGGAATGGAAtggaa | 117 |
| 10-2 | T-r2′#27 | tggaaTGGAATGGAATGgaa | 118 |
| 10-3 | T-r2′#28 | tgGaATGGAATGGAAtggaa | 119 |
| 10-4 | T-r2′#29 | tggaaTGGAATGGAATgGaa | 120 |
| 10-5 | T-r2′#30 | tggAATGGAATGGAATGgaa | 121 |
| 10-6 | T-r2′#31 | tgGaATGGAATGGAATgGaa | 122 |
| 10-7 | T-r2′#32 | tggAATGGAATGGAATgGaa | 123 |
| 10-8 | T-r2′#33 | tgGaATGGAATGGAATGgaa | 124 |

Capital letter: DNA; underlined capital letter: ENA; underlined small letter: 2′-O-MOE RNA. All the internucleoside linkages are phosphorothioate linkages.

ASOs (0.5 nM) were introduced together with a pkSCX-IRES-EGFP vector comprising an SCA31 insertion sequence in the TK2 direction into HeLa cells by a method similar to that in Example 2, and the expression levels of SCA31 mutant repeats (TK2) were quantified by qRT-PCR.

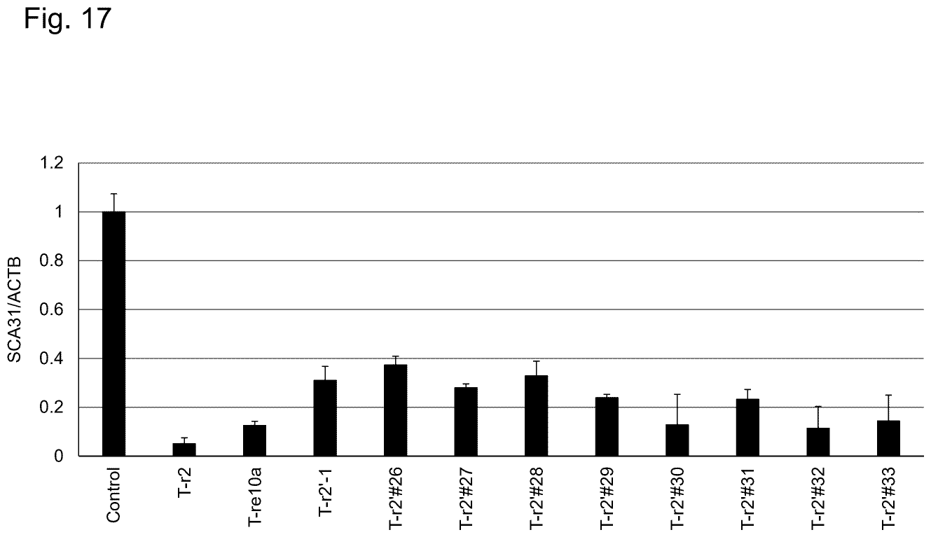

The results of the quantification of the expression levels of the SCA31 mutant repeats (TK2) are shown in FIG. 17. All of the ASOs shown in Table 9 exhibited strong effects of reducing expression, and among them, T-r2′#30, T-r2′#32, and T-r2′#33 exhibited particularly strong effects of reducing expression.

### Example 11: ASO Targeting SCA31 Mutant Repeats (TK2): MOE/ENA/DNA Gapmer

### (Purpose)

MOE/ENA/DNA gapmers in which the positions and numbers of MOEs and ENAs in a wing region are modified are synthesized, and the effect of reducing gene expression of SCA31 mutant repeats (TK2) is investigated.

### (Method and Results)

ASOs were synthesized by a method similar to that in Example 3. The synthesized ASOs are shown in Table 10.

TABLE 10

| | Compound | | SEQ ID |
|---|---|---|---|
| Example | name | Sequence (5′-3′) | NO: |
| 11-1 | T-r2′#34 | TgGaATGGAATGGAATgGaa | 125 |
| 11-2 | T-r2′#35 | tgGaATGGAATGGAATgGaA | 126 |
| 11-3 | T-r2′#36 | TGgAaTGGAATGGAATggAa | 127 |
| 11-4 | T-r2′#37 | TgGAaTGGAATGGAATggAa | 128 |
| 11-5 | T-r2′#38 | TggAATGGAATGGAATggAa | 129 |
| 11-6 | T-r2′#39 | TggAaTGGAATGGAATGgAa | 130 |
| 11-7 | T-r2′#40 | TggAaTGGAATGGAATgGAa | 131 |
| 11-8 | T-r2′#41 | TggAaTGGAATGGAATggAA | 132 |
| 11-9 | T-r2′#42 | TGGAATGGAATGGAAtggaa | 133 |
| 11-10 | T-r2′#43 | tggaaTGGAATGGAATGGAA | 134 |

Capital letter: DNA; underlined capital letter: ENA; underlined small letter: 2′-O-MOE RNA.
All the internucleoside linkages are phosphorothioate linkages.

ASOs (0.5 nM) were introduced together with a pkSCX-IRES-EGFP vector comprising an SCA31 insertion sequence in the TK2 direction into HeLa cells by a method similar to that in Example 2, and the expression levels of SCA31 mutant repeats (TK2) were quantified by qRT-PCR.

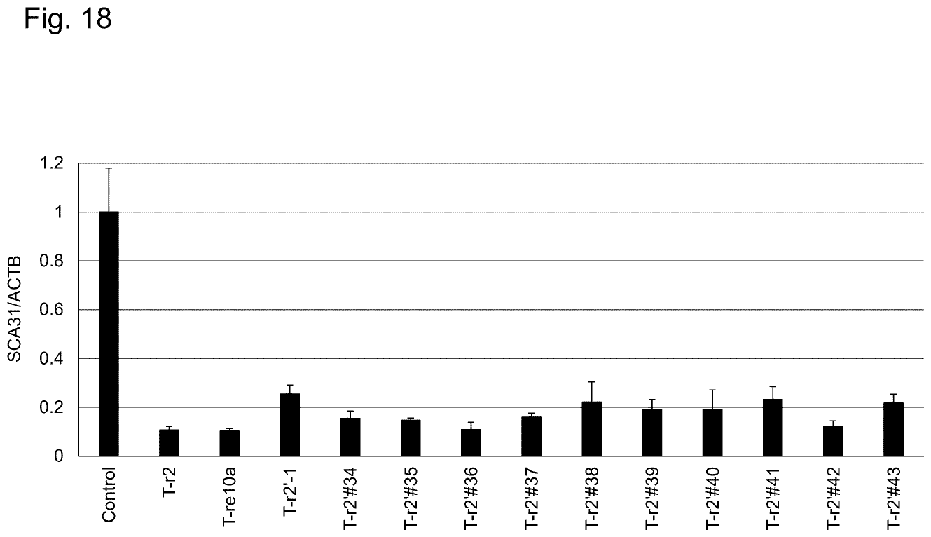

The results of the quantification of the expression levels of the SCA31 mutant repeats (TK2) are shown in FIG. 18. All of the ASOs shown in Table 10 exhibited a strong effect of reducing expression.

### Example 12: ASO Targeting SCA31 Mutant Repeats (TK2): 2′-OMe RNA/ENA/DNA Gapmer

### (Purpose)

A 2′-OMe RNA/ENA/DNA gapmer comprising 2′-OMe RNA and ENA in a wing region is synthesized, and an effect of reducing gene expression of SCA31 mutant repeats (TK2) is investigated.

### (Method and Results)

ASOs were synthesized by a method similar to that in Example 3. The synthesized ASOs are shown in Table 11.

TABLE 11

| | | | SEQ |
|---|---|---|---|
| | Compound | | ID |
| Example | name | Sequence (5′-3′) | NO: |
| 12-1 | T-r2′_1wOme | uggaaTGGAATGGAAuggaa | 135 |
| 12-2 | T-r2′#4wOme | uggAaTGGAATGGAAuggaA | 136 |
| 12-3 | T-r2′#8wOme | uggaATGGAATGGAAuggaA | 137 |
| 12-4 | T-r2′#13wOme | TggaATGGAATGGAATggaa | 138 |
| 12-5 | T-r2′#23wOme | TggAaTGGAATGGAATggAa | 139 |

Capital letter: DNA; underlined capital letter: ENA; small letter: 2′-OMe RNA.
All the internucleoside linkages are phosphorothioate linkages.

ASOs (0.5 nM) were introduced together with a pkSCX-IRES-EGFP vector comprising an SCA31 insertion sequence in the TK2 direction into HeLa cells by a method similar to that in Example 2, and the expression levels of SCA31 mutant repeats (TK2) were quantified by qRT-PCR.

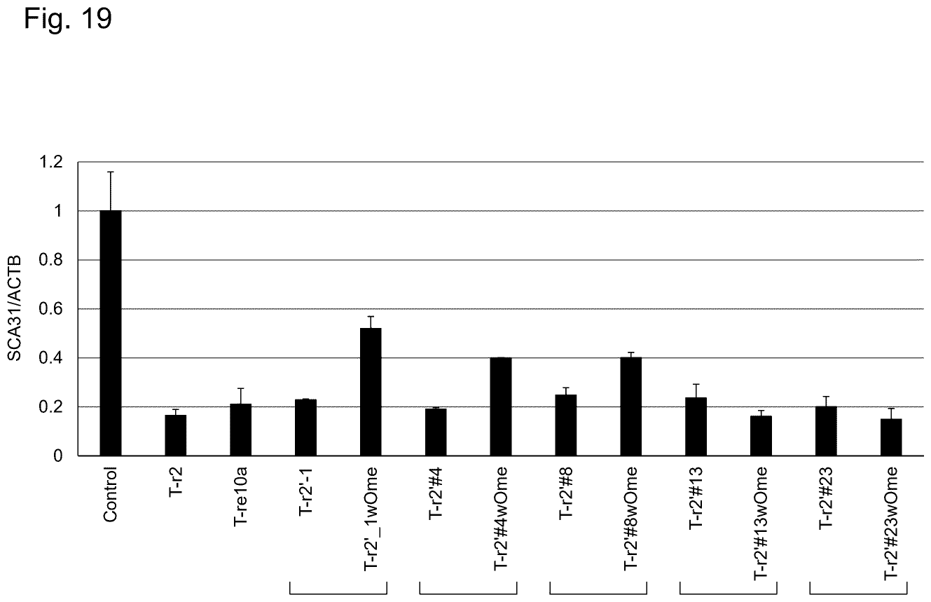

The results of the quantification of the expression levels of the SCA31 mutant repeats (TK2) are shown in FIG. 19. All of the ASOs shown in Table 11 exhibited strong effects of reducing gene expression, and among them, T-r2′#13wOme and T-r2′#23wOme exhibited strong effects of reducing gene expression.

### Example 13: Synthesis of Heteroduplex Nucleic Acid (HDO) Targeting SCA31 Mutant Repeats (TK2)

A heteroduplex nucleic acid (HDO) consisting of an ASO targeting SCA31 mutant repeats (TK2) and a complementary strand thereof is synthesized. In the ASO constituting the HDO, the number of phosphorothioate linkages (PS linkages) is reduced and phosphodiester linkages are used.

A method for synthesizing HDO is described by taking T-re10a HDO as an example.

### (1) Synthesis of TK2-r2

(SEQ ID NO: 140)
HO-C^{mls}-C^{mls}-A^{mls}-UIP-UIP-CIP-
CIP-ATP-UP-UP-Crs-C^{mls}-A^{mlt}-H

(the meaning of each symbol in the formula is as described in [Chem. 1] as described above.)
is synthesized.

The synthesis was performed by using a phosphoroamidite method (Nucleic Acids Research, 12, 4539 (1984)) using a nucleic acid automatic synthesizer (nS-8 II manufactured by Gene Design). Activator solution-3 (0.25 mol/L of 5-benzylthio-1H-tetrazole/acetonitrile solution, manufactured by Wako Pure Chemical Industries, Ltd., product No. 013-20011), CAP A for AKTA (1-methylimidazole/acetonitrile solution, manufactured by Sigma-Aldrich, product No. L040050), Cap B1 for AKTA (acetic anhydride/acetonitrile solution, manufactured by Sigma-Aldrich, product No. L050050), Cap B2 for AKTA (pyridine/acetonitrile solution, manufactured by Sigma-Aldrich, product No. L050150), DCA Deblock (dichloroacetic acid/toluene solution, manufactured by Sigma-Aldrich, product No. L023050), and Oxidizer 0.05 M Iodine for AKTA (pyridine/water/iodine=9/1/12.7 (v/v/w), manufactured by Sigma-Aldrich) were used as reagents. A solution obtained by dissolving phenylacetyl disulfide (manufactured by CARBOSYNTH, product No. FP07495) in acetonitrile (dehydrated, manufactured by KANTO CHEMICAL CO., INC., product No. 01837-05) and pyridine (dehydrated, manufactured by KANTO CHEMICAL CO., INC., product No. 11339-05) 1:1 (v/v) solution to be 0.2 M was used as a thiation reagent for forming a phosphorothioate linkage. Phosphoroamidites of RNA (adenosine body product No. ANP-5671, cytidine body product No. ANP-6676, and uridine body product No. ANP-5674) and phosphoroamidites of 2′-OMe nucleoside (adenosine body product No. ANP-5751 and cytidine body product No. ANP-5752), manufactured by ChemGenes, were used as amidite reagents. The compound of the present example was synthesized by using Primer Support 5G Unylinker 350 (manufactured by GE Healthcare) as a solid phase carrier. However, the time required for condensation of the amidite bodies was set at about 10 minutes.

An oligomer was cleaved down from a support by treating a protected oligonucleotide analog having a sequence of interest with 6 mL of concentrated aqueous ammonia/ethanol=3/1 (v/v), and a protecting cyanoethyl group on a phosphorus atom and a protecting group on a nucleobase were removed. The carrier was removed from the obtained deprotection solution by filtration, and concentrated to dryness under depressurization. A TBDMS group on the 2′-OH of RNA was removed by treating the obtained residue with 5 mL of 1.0 M TBAF solution in THF (manufactured by Tokyo Chemical Industry Co., Ltd.). To the obtained mixed solution of an oligomer, 30 mL of reaction liquid of 0.1 M TEAB solution prepared from 1.0 M TEAB solution (manufactured by Wako Pure Chemical Industries, Ltd.) was added, and the resultant was purified (Cosmosil ODS silicagel, solution A: 0.1 M TEAB solution, solution B: acetonitrile, B %: 0%→100% (16 min, linear gradient); 20 mL/min) by reverse phase open column chromatography. A fraction comprising a substance of interest was collected and concentrated under reduced pressure, and the obtained residue was treated with DOWEX 50 (manufactured by Wako Pure Chemical Industries, Ltd.). The obtained solution was concentrated under reduced pressure, 0.01 M hydrochloric acid was added thereto, and the resultant was stirred at pH 2.0 for 1 hour to remove a protecting group of 5′-OH. Concentrated aqueous ammonia was added to the obtained reaction solution, and neutralized. The obtained mixed solution of an oligomer was mixed with 30 mL of 0.1 M TEAB solution prepared from 1.0M TEAB solution (manufactured by Wako Pure Chemical Industries, Ltd.), and the mixture was charged on Clarity QSP 5 g/6 mL Cartridge (manufactured by Phenomenex). After addition of 30 mL of 0.1 M TEAB solution, a component extracted in 30 mL of 5.0, 6.6, or 10% acetonitrile/20 mM Tris aqueous solution was collected. The obtained oligomer solution was concentrated by ultrafiltration using Amicon Ultra 3K (manufactured by Merck). PBS (manufactured by Thermo Fisher) was added to perform salt exchange, and desalting was then performed to obtain a compound of interest. This compound was analyzed by reverse phase HPLC (column (Phenomenex, Clarity 2.6 μm Oligo-MS 100 A (2.1×50 mm)), solution A: 100 nM hexafluoroisopropanol (HFIP), 8 mM aqueous triethylamine solution, solution B: methanol, B %: 10%→25% (4 min)→40% (2 min)→65% (2 min)→100% (2 min), 60° C.; 0.5 mL/min; 260 nm), and then eluted at 1.44 minutes. The compound was identified (value of 4129.52, actually measured by negative ion ESI mass spectrometry) by negative ion ESI mass spectrometry.

(2) Preparation of T-Re10a HDO in which T-Re10a (SEQ ID NO: 65) and TK2-r2 (SEQ ID NO: 140) Forms a Double Strand

T-re10a (2.0 μmol, SEQ ID NO: 65) and TK2-r2 (2.0 μmol, SEQ ID NO: 140) were each dissolved in Milli Q water (1000 μL), mixed in equimolar amounts (1.8 μmol), heated at 70° C. for 5 minutes, and then ice-cooled for 5 minutes to obtain the mixed solution of the two compounds. The obtained mixed solution was subjected to electrophoresis (Tris-bolate EDTA (TBE) buffer, constant voltage of 200 V, 1 hour) using 20% polyacrylamide gel, and stained and visualized using methylene blue. As a result, a band corresponding to each of T-re10a and TK2-r2 which were single strands disappeared, and a new band of which the mobility was lower than those of T-re10a and TK2-r2 was confirmed to be generated. A compound corresponding to the newly generated band was identified as T-re10a HDO in which T-re10a and TK2-r2 formed a double strand. T-re10a HDO was obtained by lyophilizing the mixed solution of the two compounds.

Nucleic acids constituting HDOs synthesized by a method similar to the above are shown in Table 12. The configurations of nucleic acid strands constituting the HDOs are shown in Table 13.

TABLE 12

| | Compound | | SEQ ID |
|---|---|---|---|
| Example | name | Sequence (5′-3′) | NO: |
| 13-1 | TK2-r2 | 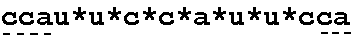 | 140 |
| 13-2 | T-re10a | TgGAATGGAATgG | 65 |
| 13-3 | T-r2′#23_0 | TggAaTGGAATGGAATggaA | 141 |
| 13-4 | T-r2′#23_1 | TggAaT*GGA*A*TG*G*AATggaA | 142 |
| 13-5 | T-r2′#23_2 | TggAaT*G*GA*A*TG*G*AATggaA | 143 |
| 13-6 | T-r2′#23_3 | TggAaT*GG*AA*TG*GA*ATggaA | 144 |
| 13-7 | T-r2′#23_4 | TggAaTGGA*A*T*G*G*A*ATggaA | 145 |
| 13-8 | T-r2′#23_5 | TggAaTGGAA*T*G*G*A*ATggaA | 146 |
| 13-9 | T-r2′#23_6 | Tgg*A*aTGGAATGGAAT*g*gaA | 147 |
| 13-10 | T-r2′#23_7 | T*gg*A*aTGGAATGGAAT*g*gaA | 148 |
| 13-11 | T-r2′#23_8 | Tgg*A*a*TGGAATGGAATg*gaA | 149 |
| 13-12 | T-r2′#23_9 | Tg*gA*aTGGAATGGAAT*gg*aA | 150 |
| 13-13 | TK2-r3 | 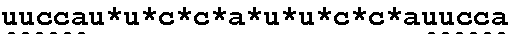 | 151 |

Capital letter: DNA; underlined capital letter: ENA; small letter: RNA; small letter with dotted underline: 2′-OMe RNA. Regarding the internucleoside linkages, symbols ″*″ show phosphodiester linkages, and all the other linkages are phosphorothioate linkages.

TABLE 13

| | | Compound | | SEQ ID |
|---|---|---|---|---|
| Example | HDO name | name | Sequence (5′-3′) | NO: |
| 13-14 | T-re10a | T-re10a | TgGAATGGAATqG | 65 |
| | HDO | TK2-r2 | 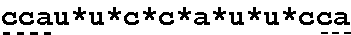 | 140 |
| 13-15 | T-r2′#23_0 | T-r2′#23_0 | TggAaTGGAATGGAATggaA | 141 |
| | HDO | TK2-r3 | 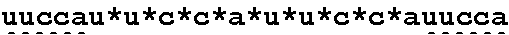 | 151 |
| 13-16 | T-r2′#23 1 | T-r2′#23 1 | TggAaT*GGA*A*TG*G*AATggaA | 142 |
| | HDO | TK2-r3 | 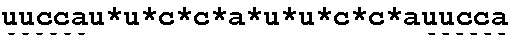 | 151 |
| 13-17 | T-r2′#23_2 | T-r2′#23_2 | TggAaT*G*GA*A*TG*G*AATggaA | 143 |
| | HDO | TK2-r3 | 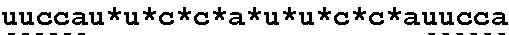 | 151 |
| 13-18 | T-r2′#23 3 | T-r2′#23 3 | TggAaT*GG*AA*TG*GA*ATggaA | 144 |
| | HDO | TK2-r3 | 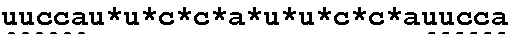 | 151 |
| 13-19 | T-r2′#23 4 | T-r2′#23_4 | TggAaTGGA*A*T*G*G*A*ATggaA | 145 |
| | HDO | TK2-r3 | 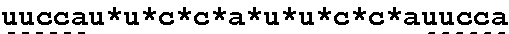 | 151 |
| 13-20 | T-r2′#23_5 | T-r2′#23 5 | TggAaTGGAA*T*G*G*A*ATggaA | 146 |
| | HDO | TK2-r3 | 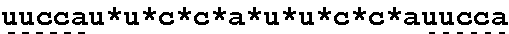 | 151 |
| 13-21 | T-r2′#23 6 | T-r2′#23_6 | Tgg*A*aTGGAATGGAAT*g*gaA | 147 |
| | HDO | TK2-r3 | 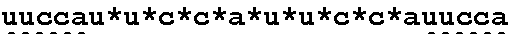 | 151 |
| 13-22 | T-r2'#23_7 | T-r2′#23_7 | T*gg*A*aTGGAATGGAAT*g*gaA | 148 |
| | HDO | TK2-r3 | 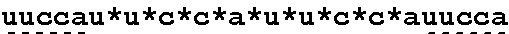 | 151 |
| 13-23 | T-r2′#23 8 | T-r2′#23_8 | Tgg*A*a*TGGAATGGAATg*gaA | 149 |
| | HDO | TK2-r3 | 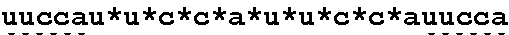 | 151 |
| 13-24 | T-r2′#23_9 | T-r2′#23 9 | Tg*gA*aTGGAATGGAAT*gg*aA | 150 |
| | HDO | TK2-r3 | 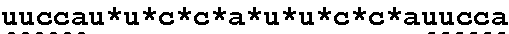 | 151 |

Capital letter: DNA; underlined capital letter: ENA; small letter: RNA; small letter with dotted underline: 2′-OMe RNA; underlined small letter: 2′-O-MOE RNA. Regarding the internucleoside linkages, symbols ″*″ show phosphodiester linkages, and all the other linkages are phosphorothioate linkages.

### Example 14: Investigation of Effect of Reducing Gene Expression by HDO Synthesized in Example 13

### (Purpose)

The effect of reducing gene expression of SCA31 mutant repeats (TK2) by HDO synthesized in Example 13 is investigated.

### (Method and Results)

HDOs (0.5 nM) were introduced together with a pkSCX-IRES-EGFP vector comprising an SCA31 insertion sequence in the TK2 direction into HeLa cells by a method similar to that in Example 2, and the expression levels of SCA31 mutant repeats (TK2) were quantified by qRT-PCR.

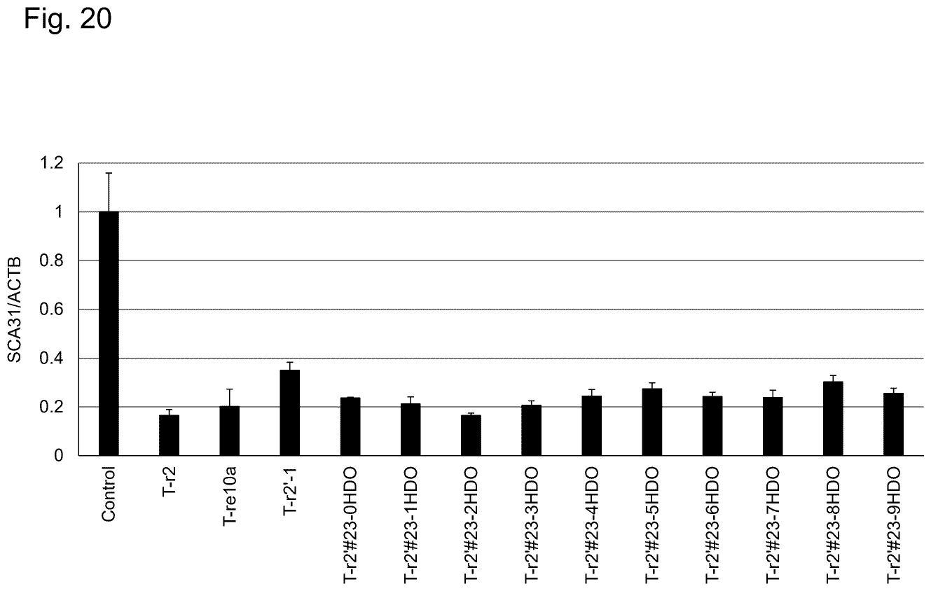

The results of the quantification of the expression levels of the SCA31 mutant repeats (TK2) are shown in FIG. 20. All of the HDOs shown in Table 13 exhibited strong effects of reducing expression.

### Example 15: Mixmer Targeting SCA31 Mutant Repeats (TK2)

### (Purpose)

A mixmer targeting SCA31 mutant repeats (TK2) is synthesized, and the effect of reducing gene expression of the SCA31 mutant repeats (TK2) is investigated.

### (Method and Results)

ASOs were synthesized by a method similar to that in Example 3. The synthesized ASOs are shown in Table 14.

TABLE 14

| | | | SEQ |
|---|---|---|---|
| | | Sequence | ID |
| Example | Compound name | (5′-3′) | NO: |
| 15-1 | T-r1-LNA/DNA | GGAATGG | 152 |
| | gapmer (T-LDG) | AATGGA | |
| 15-2 | T-r1-LNA/RNA | GGaatgg | 153 |
| | gapmer (T-LRG) | aauGGA | |
| 15-3 | T-r1-LNA/DNA | GGAATGG | 154 |
| | mixmer (T-LDM) | AATGGA | |
| 15-4 | T-r1-LNA/RNA | GgaaTgg | 155 |
| | mixmer (T-LRM) | AaugGA | |

Capital letter: DNA; underlined capital letter: LNA; small letter: RNA. All the
internucleoside linkages are phosphorothioate linkages.

ASOs (2 nM) were introduced together with a pkSCX-IRES-EGFP vector comprising an SCA31 insertion sequence in the TK2 direction into HeLa cells by a method similar to that in Example 2, and the expression levels of SCA31 mutant repeats (TK2) were quantified by qRT-PCR.

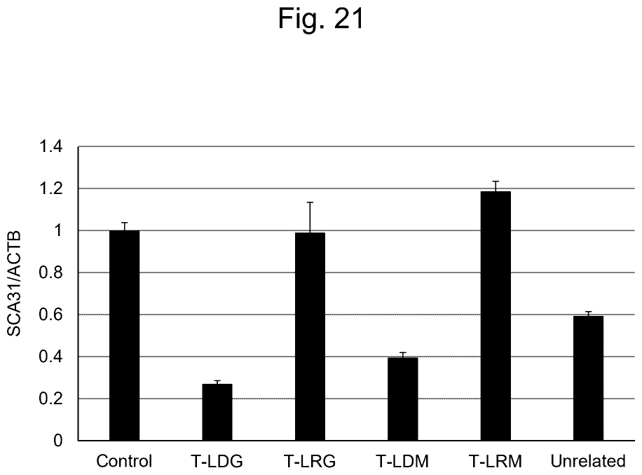

The results of the quantification of the expression levels of the SCA31 mutant repeats (TK2) are shown in FIG. 21. Among mixmers shown in Table 14, the T-r1-LNA/DNA mixmer (T-LDM) produces a favorable effect of reducing expression.

### Example 16: ASO (LNA/DNA Gapmer) Targeting SCA31 Mutant Repeats (BEAN1)

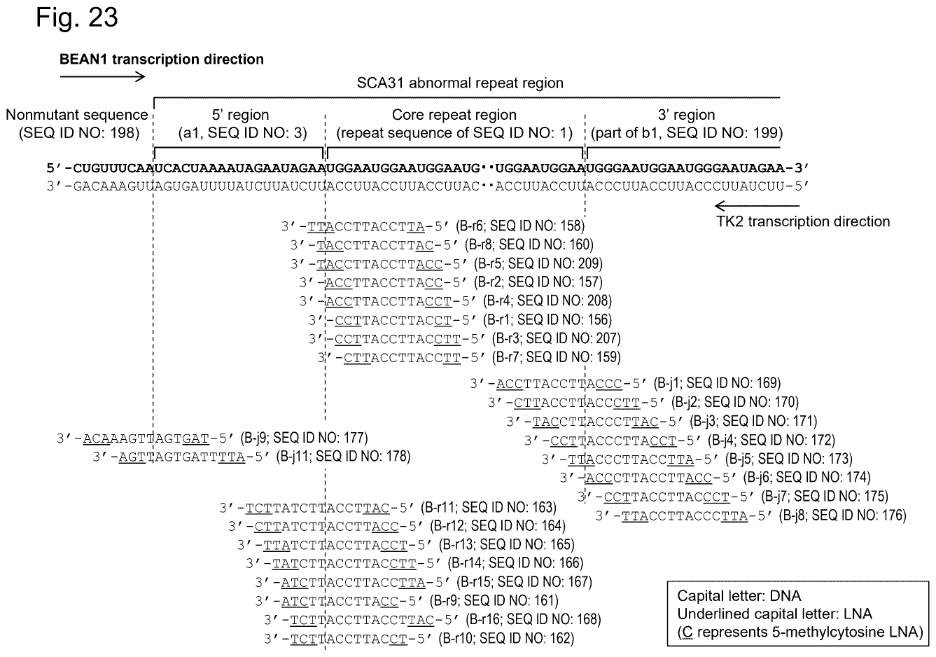

ASOs (LNA/DNA gapmers) targeting an SCA31 abnormal repeat region in a mutant BEAN1 gene transcriptional product (hereinafter referred to as “SCA31 mutant repeat (BEAN1)”) were synthesized by a method similar to that in Example 1. The synthesized nucleic acids are shown in the following Table 15 and FIG. 23.

TABLE 15

| | Compound | | SEQ ID |
|---|---|---|---|
| Example | name | Sequence (5′-3′) | NO: |
| 16-1 | B-r1 | TCCATTCCATTCC | 156 |
| 16-2 | B-r2 | CCATTCCATTCCA | 157 |
| 16-3 | B-r3 | TTCCATTCCATTCC | 207 |
| 16-4 | B-r4 | TCCATTCCATTCCA | 208 |
| 16-5 | B-r5 | CCATTCCATTCCAT | 209 |
| 16-6 | B-r6 | ATTCCATTCCATT | 158 |
| 16-7 | B-r7 | TTCCATTCCATTC | 159 |
| 16-8 | B-r8 | CATTCCATTCCAT | 160 |
| 16-9 | B-r9 | CCATTCCATTCTA | 161 |
| 16-10 | B-r10 | TCCATTCCATTCT | 162 |
| 16-11 | B-r11 | CATTCCATTCTATTCT | 163 |
| 16-12 | B-r12 | CCATTCCATTCTATTC | 164 |
| 16-13 | B-r13 | TCCATTCCATTCTATT | 165 |
| 16-14 | B-r14 | TTCCATTCCATTCTAT | 166 |
| 16-15 | B-r15 | ATTCCATTCCATTCTA | 167 |
| 16-16 | B-r16 | CATTCCATTCCATTCT | 168 |
| 16-17 | B-j1 | CCCATTCCATTCCA | 169 |
| 16-18 | B-j2 | TTCCCATTCCATTC | 170 |
| 16-19 | B-j3 | CATTCCCATTCCAT | 171 |
| 16-20 | B-j4 | TCCATTCCCATTCC | 172 |
| 16-21 | B-j5 | ATTCCATTCCCATT | 173 |
| 16-22 | B-j6 | CCATTCCATTCCCA | 174 |
| 16-23 | B-j7 | TCCCATTCCATTCC | 175 |
| 16-24 | B-j8 | ATTCCCATTCCATT | 176 |
| 16-25 | B-j9 | TAGTGATTGAAACA | 177 |
| 16-26 | B-j11 | ATTTTAGTGATTGA | 178 |

Capital letter: DNA; underlined capital letter: LNA.
All the internucleoside linkages are phosphorothioate linkages.
The nucleobase C in LNA is 5-methylcvtosine.

Transient expression of SCA31 mutant repeats (BEAN1) is conducted, the produced ASOs are introduced into HeLa cells, and the effect of reducing gene expression of the SCA31 mutant repeats (BEAN1) is investigated. For the transient expression of the SCA31 mutant repeats (BEAN1), the sequences of the SCA31 mutant repeats (BEAN1) were positioned downstream of a chicken R-actin promoter in a manner similar to that in Example 2, and a pkSCX-IRES-EGFP vector having IRES-EGFP was used just after them (FIG. 36B). ASOs (2 nM) were introduced together with a pkSCX-IRES-EGFP vector comprising an SCA31 insertion sequence in the BEAN1 direction into HeLa cells by a manner similar to that in Example 2, and the expression levels of SCA31 mutant repeats (BEAN1) were quantified by qRT-PCR. In qRT-PCR, a probe primer mixture: SCA31-0 (TaqMan) consisting of SCA31-0_primer FW (SEQ ID NO: 18, 5′-TGGCTGCACATAGCTTTATCTCTT-3′) and SCA31-0_primer RV (SEQ ID NO: 20, 5′-AAGCCCAATCTGGAAGCAAA-3′) was used.

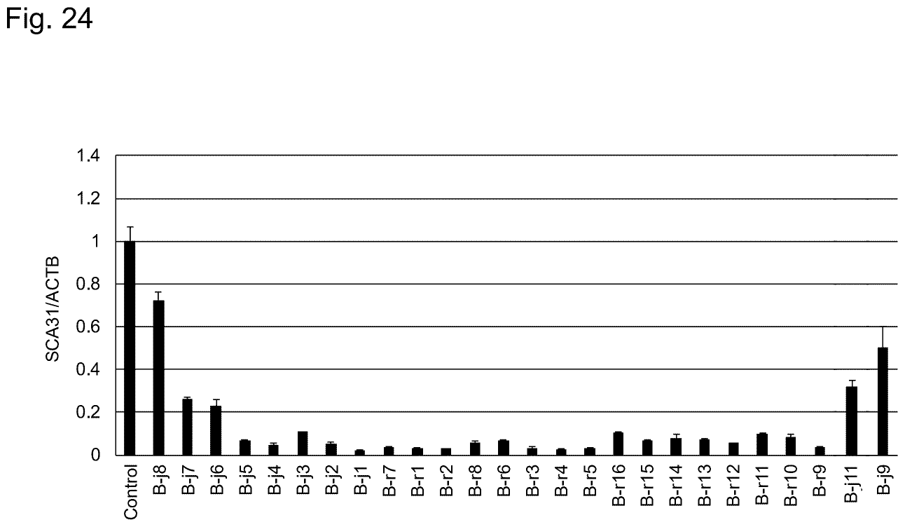

The results of the quantification of the expression levels of the SCA31 mutant repeats (BEAN1) are shown in FIG. 24. All of the compounds used for the evaluation exhibited strong effects of reducing gene expression. Strong effects of reducing expression of the SCA31 mutant repeats (BEAN1) were obtained particularly by B-r11, B-r12, B-r13, B-r14, B-r15, B-r16, B-r9, B-r10, B-r6, B-r8, B-r5, B-r4, B-r3, B-r2, B-r1, B-r7, B-j1, B-j2, B-j3, B-j4, and B-j5. The results revealed that the strong effect of reducing expression tended to be obtained when the target sequence of ASO comprises the boundary between the 5′ region and the core repeat region, when the target sequence of ASO comprises the boundary between the core repeat region and the 3′ region, or when the target sequence of ASO is positioned in the interior side of the core repeat region.

The inhibition of expression of the SCA31 mutant repeats (BEAN1) by the ASOs targeting the SCA31 mutant repeats (BEAN1) was observed by FISH similar to that in Example 2, using an LNA-(TTCCA)₅ probe.

### Example 18: ASO Targeting SCA31 Mutant Repeats (BEAN1): Surrounding Sequence of B-r2

### (Purpose)

ASO consisting of the surrounding sequence of B-r2 exhibiting a strong effect of reducing expression of the SCA31 mutant repeats (BEAN1) in Example 16 is synthesized, and the effect is investigated.

### (Method and Results)

ASOs were synthesized by a method similar to that in Example 3. The synthesized ASOs are shown in Table 16.

TABLE 16

| | Compound | | SEQ ID |
|---|---|---|---|
| Example | name | Sequence (5′-3′) | NO: |
| 18-1 | B-r2a | AuTCCATTCCATTCcA | 179 |
| 18-2 | B-r2b | TuCCATTCCATTCCaT | 180 |

Capital letter: DNA; small letter: 2′-OMe RNA; underlined capital letter: LNA.
All the internucleoside linkages are phosphorothioate linkages.
The nucleobase C in LNA is 5-methylcytosine.

ASOs (0.1 nM) were introduced together with a pkSCX-IRES-EGFP vector comprising an SCA31 insertion sequence in the BEAN1 direction into HeLa cells by a method similar to that in Example 16, and the expression levels of SCA31 mutant repeats (BEAN1) were quantified by qRT-PCR.

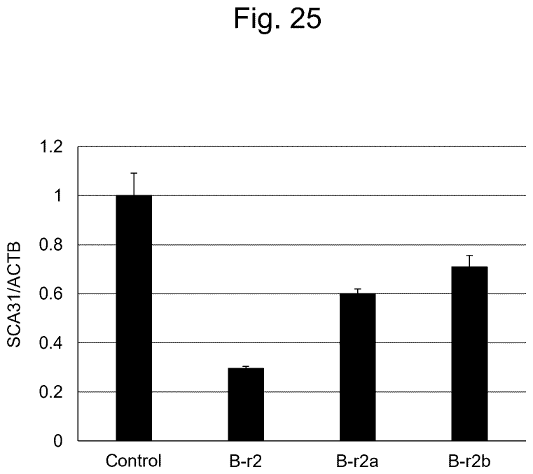

The results of the quantification of the expression levels of the SCA31 mutant repeats (BEAN1) are shown in FIG. 25. Both B-r2a and B-r2b exhibited strong effects of reducing gene expression. The effects of reducing expression of B-r2 was higher than the effects of B-r2a and B-r2b.

### Example 19: ASO Targeting Interior Part of Core Repeat Region of SCA31 Mutant Repeat (BEAN1): MOE/DNA Gapmer

### (Purpose)

ASOs (MOE/DNA gapmers) targeting the interior part of the core repeat region of SCA31 mutant repeats (BEAN1) are synthesized, and the effect of reducing gene expression of the SCA31 mutant repeats (BEAN1) is investigated.

### (Method and Results)

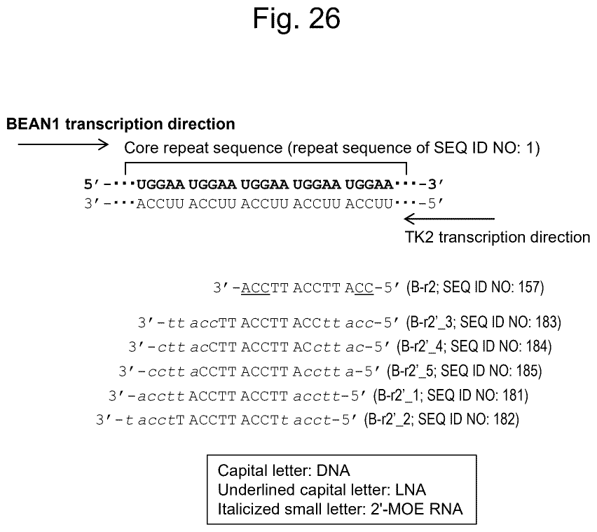

ASOs were synthesized by a method similar to that in Example 3. The synthesized ASOs are shown in Table 17. The position of each ASO in the SCA31 mutant repeats (BEAN1) is shown in FIG. 26.

TABLE 17

| | Compound | | SEQ ID |
|---|---|---|---|
| Example | name | Sequence (5′-3′) | NO: |
| 19-1 | B-r2′_1 | ttccaTTCCATTCCAttcca | 181 |
| 19-2 | B-r2′ 2 | tccatTCCATTCCATtccat | 182 |
| 19-3 | B-r2′_3 | ccattCCATTCCATTccatt | 183 |
| 19-4 | B-r2′_4 | cattcCATTCCATTCcattc | 184 |
| 19-5 | B-r2′_5 | attccATTCCATTCCattcc | 185 |

Capital letter: DNA; underlined small letter: 2′-O-MOE RNA.
All the internucleoside linkages are phosphorothioate linkages.
The nucleobase C in 2′-O-MOE RNA is 5-methylcytosine.

ASOs (0.1 nM) were introduced together with a pkSCX-IRES-EGFP vector comprising an SCA31 insertion sequence in the BEAN1 direction into HeLa cells by a method similar to that in Example 16, and the expression levels of SCA31 mutant repeats (BEAN1) were quantified by qRT-PCR.

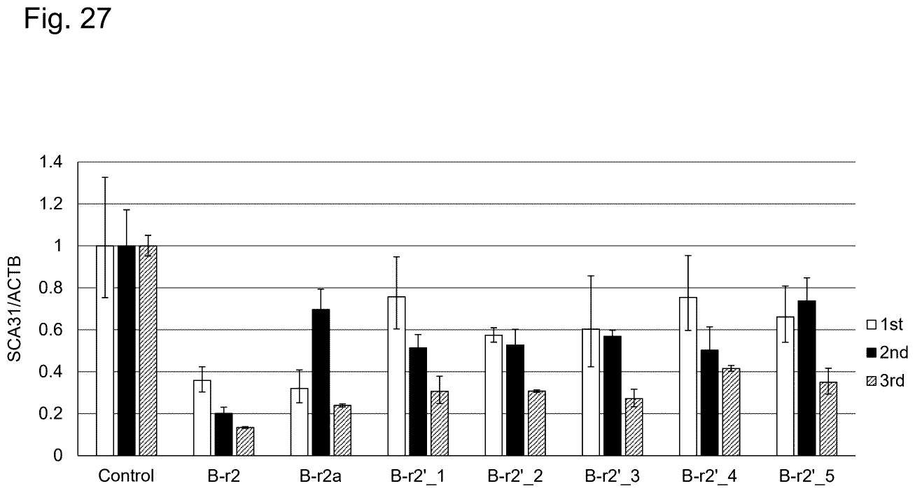

The results of the quantification of the expression levels of the SCA31 mutant repeats (BEAN1) are shown in FIG. 27. All of the ASOs shown in Table 17 exhibited favorable effects of reducing expression.

### Example 20: Mixmer Targeting SCA31 Mutant Repeat (BEAN1)

### (Purpose)

A mixmer targeting SCA31 mutant repeats (BEAN1) is synthesized, and the effect of reducing gene expression of the SCA31 mutant repeats (BEAN1) is investigated.

### (Method and Results)

ASOs were synthesized by a method similar to that in Example 3. The synthesized ASOs are shown in Table 18.

TABLE 18

| | | Sequence | SEQ ID |
|---|---|---|---|
| Example | Compound name | (5′-3′) | NO: |
| 20-1 | B-r1-LNA/RNA | TCCATTC | 186 |
| | gapmer(B-LDG) | CATTCC | |
| 20-2 | B-r1-LNA/RNA | TCcattc | 187 |
| | gapmer(B-LRG) | cauTCC | |
| 20-3 | B-r1-LNA/DNA | TCCATTC | 188 |
| | mixmer(B-LDM) | CATTCC | |
| 20-4 | B-r1-LNA/RNA | TccaTtc | 189 |
| | mixmer(B-LRM) | CattCC | |

Capital letter: DNA; small letter: 2′-OMe RNA; underlined capital letter: LNA.
All the internucleoside linkages are phosphorothioate linkages.
The nucleobase C in LNA is 5-methylcytosine.

ASOs (2 nM) were introduced together with a pkSCX-IRES-EGFP vector comprising an SCA31 insertion sequence in the BEAN1 direction into HeLa cells by a method similar to that in Example 16, and the expression levels of SCA31 mutant repeats (BEAN1) were quantified by qRT-PCR.

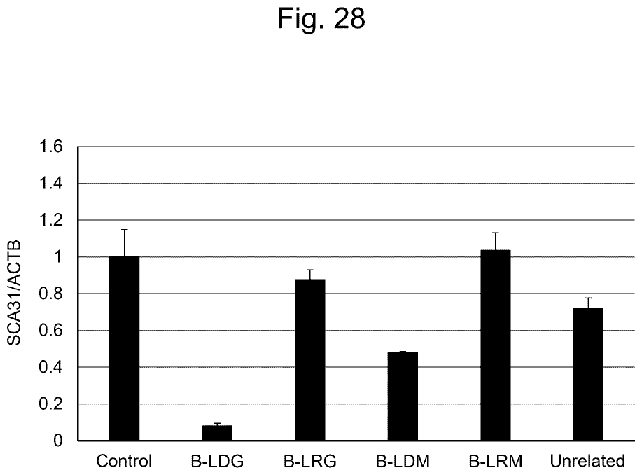

The results of the quantification of the expression levels of the SCA31 mutant repeats (BEAN1) are shown in FIG. 28. Among the mixmers shown in Table 18, the B-r1-LNA/DNA mixmer (B-LDM) exhibited a comparatively favorable effect of reducing expression.

### Example 21: Investigation of Effect of Reducing Expression of SCA31 Mutant Repeat In Vivo

### (Purpose)

The effect of reducing gene expression of SCA31 mutant repeats (TK2) and SCA31 mutant repeats (BEAN1) by ASO/HDO is investigated in vivo.

### (Method and Results)

### (1) Production of SCA31 Disease Model Mice

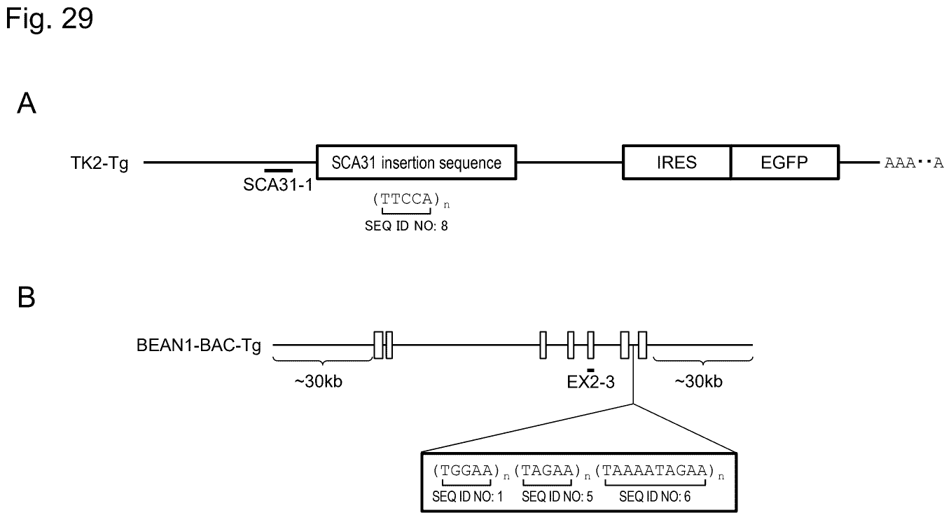

TK2-Tg mice and BEAN1-BAC-Tg mice were produced in order to examine the effect of ASO in vivo. The TK2-Tg mice were produced by introducing, into C57BL/6J mice, the same construct (FIG. 29A) as the pkSCX-IRES-EGFP vector comprising the SCA31 insertion sequence in the TK2 direction, used in the examples described above. The line D produced by introducing, into the C57BL/6N mice, BAC (FIG. 29B) encompassing a patient genome region was used as BEAN1-BAC Tg. All the Tg mice expressed a mutant sequence similar to that in the brain of a patient with SCA31, and showed abnormal gait.

General description of the TK2-Tg mice and BEAN-BAC-Tg mice used in the present example is shown in the following Table 19.

TABLE 19

| | | | Expression of | |
|---|---|---|---|---|
| | | | RNA/formation | |
| Name of Tg | | Expression | of RNA | Gait |
| mouse | Transgene | site | aggregate | disturbance*¹ |
| TK2-Tg | Construct comprising SCA31 | Only brain | Yes | Observed in |
| | mutant repeats (TK2) derived | | | 68-week-old |
| | from patient | | | mouse |
| BEAN1 BAC- | BAC encompassing patient | All cells | Yes | Observed in |
| Tg | genome region | | | 68-week-old |
| | | | | mouse |

*¹The result of rotarod test, a treadmill test, and the like showed gait disturbance.

(2) Injection into Cerebral Ventricle of Mouse

ASOs/HDOs were injected into the cerebral ventricles of the TK2-Tg mice (passage number: F5, 13-week-old, body weight: 20 to 30 g) and BEAN1-BAC Tg mice (passage number: F9, 25-week-old, body weight: 20 to 30 g) produced in (1).

The ASO was modified at 85° C. for 3 minutes using 50 μg/μL of PBS as a stock solution, and then used. For the HDO, cRNA of which the amount was equal to that of the main chain was prepared at 95° C. for 5 minutes, and at 37° C. for 60 minutes. The ASO was diluted with PBS to form 10 μL of agent liquid. The HDO was diluted with PBS to form 10 μL of agent liquid. As a control, 10 μL of PBS was administered.

For anesthesia of the mice, 2% of isoflurane was introduced, and 1.5 to 2.5% was maintained during operation. The temperature was kept at 38° C. during and after the operation.

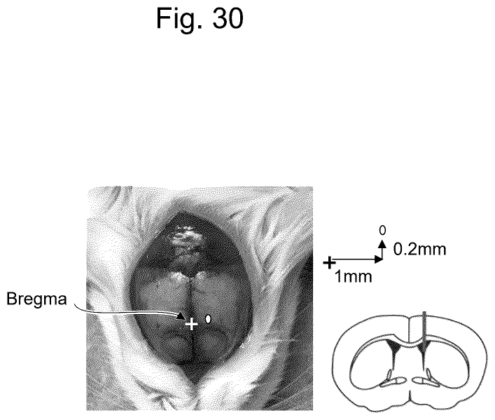

For the injection into the cerebral ventricle of each mouse, the injection into the left cerebral ventricle was performed. The position of the injection was set at the position of 3 mm intracranially from the site of 1 mm outward and 0.2 mm caudally from the bregma (indicated by “∘” in FIG. 30). Puncture was performed, the needle was left to stand for 2 minutes, injection of 10 μL of the agent was then performed for 3 minutes, and the needle was left to stand for 5 minutes and then removed.

The ASOs and HDOs used in the present examples are each shown in Tables 20 to 23.

TABLE 20

| | Compound | Sequence | SEQ ID |
|---|---|---|---|
| Example | name | (5′-3′) | NO: |
| 13-2 | T-re10a | TgGAATGGAATgG | 65 |

Capital letter: DNA; small letter: 2′-OMe RNA; underlined capital letter: ENA.
All the internucleoside linkages are phosphorothioate linkages.

TABLE 21

| | Compound | | SEQ ID |
|---|---|---|---|
| Example | name | Sequence (5′-3′) | NO: |
| 8-1 | T-r2′_1 | tggaaTGGAATGGAAtggaa | 87 |

Capital letter: DNA; underlined small letter: 2′-O-MOE RNA.
All the internucleoside linkages are phosphorothioate linkages.

TABLE 22

| | Compound | Sequence | SEQ ID |
|---|---|---|---|
| Example | name | (5′-3′) | NO: |
| 16-2 | B-r2 | CCATTCCATTCCA | 157 |

Capital letter: DNA; underlined capital letter: LNA.
All the internucleoside linkages are phosphorothioate linkages.
The nucleobase C in LNA is 5-methylcytosine.

TABLE 23

| | Name of | Name of | | SEQ ID |
|---|---|---|---|---|
| Example | HDO | oligonucleotide | Sequence (5′-3′) | NO: |
| 13-13 | T-re10a | T-re10a | TgGAATGGAATgG | 65 |
| | HDO | TK2-r2 | 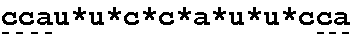 | 140 |

Capital letter: DNA; underlined capital letter: ENA; small letter: RNA; small letter with dotted underline: 2′-OMe RNA. Regarding the internucleoside linkages, symbols ″*″ show phosphodiester linkages, and all the other linkages are phosphorothioate linkages.

(3) Expression Analysis by qRT-PCR

The bilateral hippocampus and the cerebellum were isolated 1 week after the injection into the cerebral ventricle. Extraction was performed using Trizol, and RNA was collected by an isopropanol sedimentation method. From 1 μg of the collected RNA, cDNA was prepared using PrimeScript Takara. A 10-fold dilution of a product obtained from each TK2-Tg mouse and a 5-fold dilution of a product obtained from each BEAN1-BAC-Tg mouse by EASY Dilusion were used as templates. By Roche Light Cycler 48011, q-PCR was performed. As primers for q-PCR, SCA31-1 (TaqMan) consisting of SCA31-1_primer FW (SEQ ID NO: 10, 5′-TGGATAGAAACCCCAGCTTCCT-3′) and SCA31-1_primer RV (SEQ ID NO: 15, 5′-CTGCCTCCGACACTTTTCAATT-3′) was used for the TK2-Tg mice, and AB472391-1 (Ex2-3) consisting of Ex2-3_primer FW (SEQ ID NO: 25, 5′-CAGCCACTGCCCAGGACTAA-3′) and Ex2-3_primer RV (SEQ ID NO: 30, 5′-GCCACTTCTCTGCCTCAGAGA-3′) was used for the BEAN1 BAC-Tg mice. The positions detected by q-PCR are shown as “SCA31-1” and “EX2-3” in FIG. 29. ACTB (Mm00607939_s1) was used as a reference.

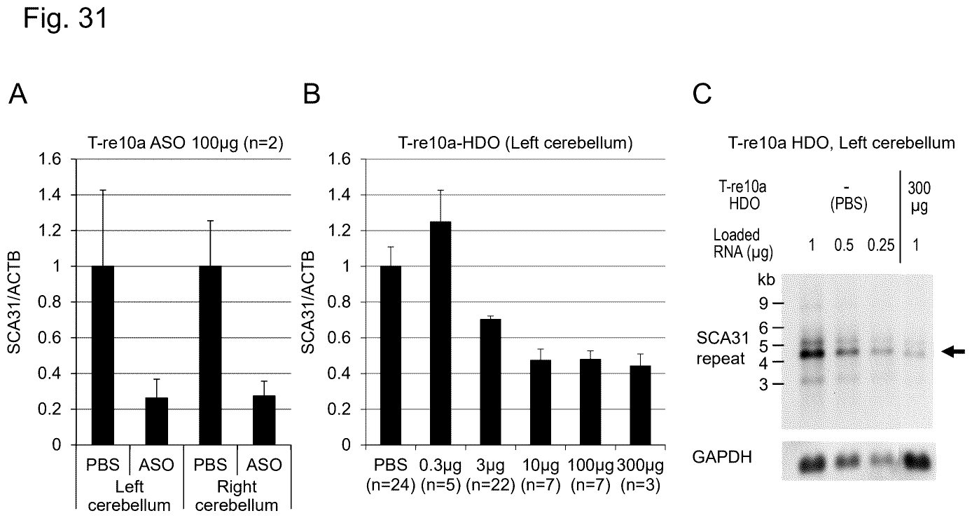

The results of reducing expression of SCA31 mutant repeats (TK2) by T-re10a ASO (100 μg) are shown in FIG. 31A. The strong effect of reducing expression of the SCA31 mutant repeats was found to be obtained in vivo by T-re10a ASO.

The results of reducing expression of SCA31 mutant repeats (TK2) by T-re10a HDO (0.3 μg to 300 μg) are shown in FIG. 31B. The favorable effect of reducing expression of the SCA31 mutant repeats was found to be obtained in vivo in a concentration-dependent manner by T-re10a HDO. The administration of 10 μg or more particularly resulted in the strong effect.

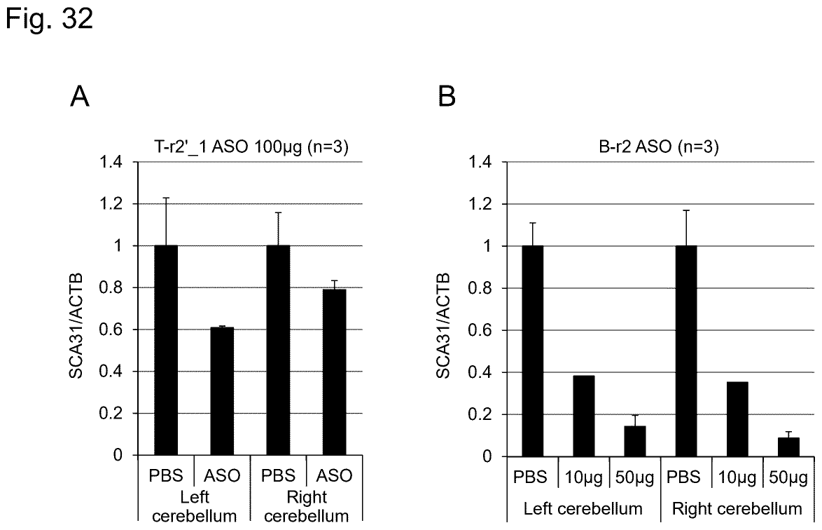

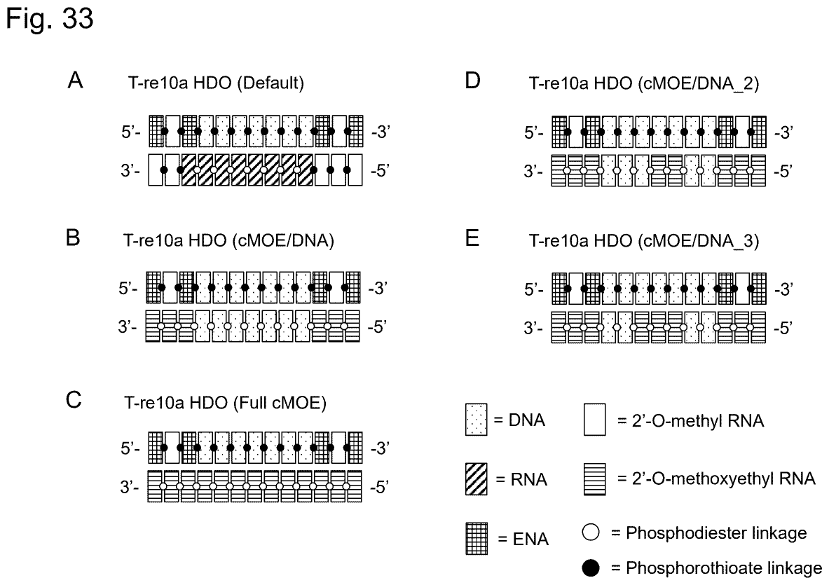

The results of reducing expression of SCA31 mutant repeats (TK2) by T-r2′_1 ASO (100 μg) are shown in FIG. 32A. The effect of reducing expression of the SCA31 mutant repeats was found to be also obtained in vivo by T-r2′_1 ASO although the effect was weaker than that by T-re10a ASO.

The results of reducing expression of SCA31 mutant repeats (BEAN1) by B-r2 ASO (10 μg or 50 μg) are shown in FIG. 32B. The strong effect of reducing expression of the SCA31 mutant repeats (BEAN1) was produced by administering 50 μg of B-r2 ASO.

### (4) Analysis of Expression by Northern Blot

A sample obtained by performing DNase treatment of an RNA sample obtained from the left cerebellum by a method similar to that in (3) and then performing polyA purification of the RNA sample using Oligotex (trademark) -dt30<super> mRNA Purification Kit Takara was subjected to Northern blot. The sample was electrophoresed at 50 V for 3.5 hours, and transferred at 20 hours, followed by fixing RNA on a membrane by UV cross-link. Hybridization was performed at 45° C. in O/N using LNA-(TTCCA)₅ probe (SEQ ID NO: 31) and LNA-(TGGAA)₅ probe (SEQ ID NO: 32) labelled with DIG, and detection with an anti-DIG-AP antibody was performed. Coloring was performed with CDP Star using DIG Wash and Block Buffer Set.

The results of the Northern blot are shown in FIG. 31C. In FIG. 31C, the first to third lanes from the left show the results of loading each of 1 μg, 0.5 μg, and 0.25 μg of RNA samples obtained from the negative control mice injected with PBS and detecting SCA31 repeats. The fourth lane from the left shows the result of loading 1 μg of an RNA sample obtained from the mouse injected with 300 μg of T-re10a HDO and detecting SCA31 repeats. The results showed that the expression of the SCA31 mutant repeats (TK2) was decreased to ¼ or less by administering T-re10a HDO.

### Example 22: HDO Comprising 2′-O-MOE-RNA Nucleoside

### (Purpose)

HDO in which a first nucleic acid strand consists of T-re10a and a second nucleic acid strand comprises 2′-O-MOE-RNA nucleoside is produced, and the effect of reducing gene expression of SCA31 mutant repeats (TK2) is investigated.

### (Method and Results)

HDOs were produced by a method similar to that in Example 21. The produced HDOs are shown in the following Table 24.

TABLE 24

| | | | | SEQ ID |
|---|---|---|---|---|
| Example | Name of HDO | Compound name | Sequence (5′-3′) | NO: |
| 22 | T-re10a HDO | T-re10a | TgGAATGGAATgG | 65 |
| | (Default) | TK2-r2 | 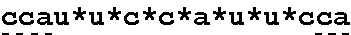 | 140 |
| 22 | T-re10a HDO | T-re10a | TgGAATGGAATgG | 65 |
| | (cMOE/DNA) | TK2-r2 | c(5)*c(5)*a*T*T*C*C*A*T*T | 200 |
| | | (cMOE/DNA) | *c(5)*c(5)*a | |
| 22 | T-re10a HDO | T-re10a | TgGAATGGAATgG | 65 |
| | (Full cMOE) | TK2-r2 | c(5)*c(5)*a*T*T*c(5)*c(5) | 201 |
| | | (Full cMOE) | *a*t*t*c(5)*c(5)*a | |
| 22 | T-re10a HDO | T-re10a | TgGAATGGAATgG | 65 |
| | (cMOE/DNA_2) | TK2-r2 | c(5)*c(5)*a*T*T*c(5)*c(5) | 202 |
| | | (cMOE/DNA_2) | *A*T*T*c(5)*c(5)*a | |
| 22 | T-re10a HDO | T-re10a | TgGAATGGAATgG | 65 |
| | (cMOE/DNA_3) | TK2-r2 | c(5)*c(5)*a*T*T*c(5)*c(5) | 203 |
| | | (cMOE/DNA_3) | *a*T*T*c(5)*c(5)*a | |

Capital letter: DNA; underlined capital letter: ENA; small letter: RNA; small letter with dotted underline: 2′-OMe RNA; underlined small letter: 2′-O-MOE RNA (c(5) represents 5-methylcytosine 2′-O-MOE-RNA). Regarding the internucleoside linkages, symbols ″*″ show phosphodiester linkages, and all the other linkages are phosphorothioate linkages.

HDOs were injected into the cerebral ventricles of TK2-Tg mice by a method similar to that in Example 21, and 1 week later the expression of SCA31 mutant repeats (TK2) in the bilateral hippocampus and the cerebellum was analyzed by qRT-PCR.

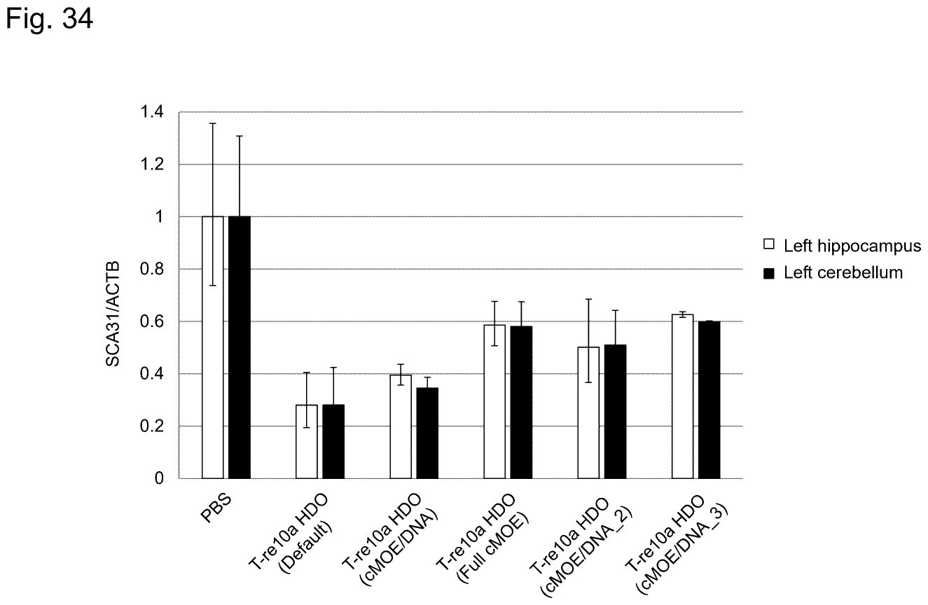

The results of reducing expression of SCA31 mutant repeats (TK2) by T-re10a HDO (Default), T-re10a HDO (cMOE/DNA), T-re10a HDO (Full cMOE), T-re10a HDO (cMOE/DNA_2), and T-re10a HDO (cMOE/DNA_3) are shown in FIG. 34. The strong effect of reducing expression of the SCA31 mutant repeats was found to be produced in vivo by each HDO.

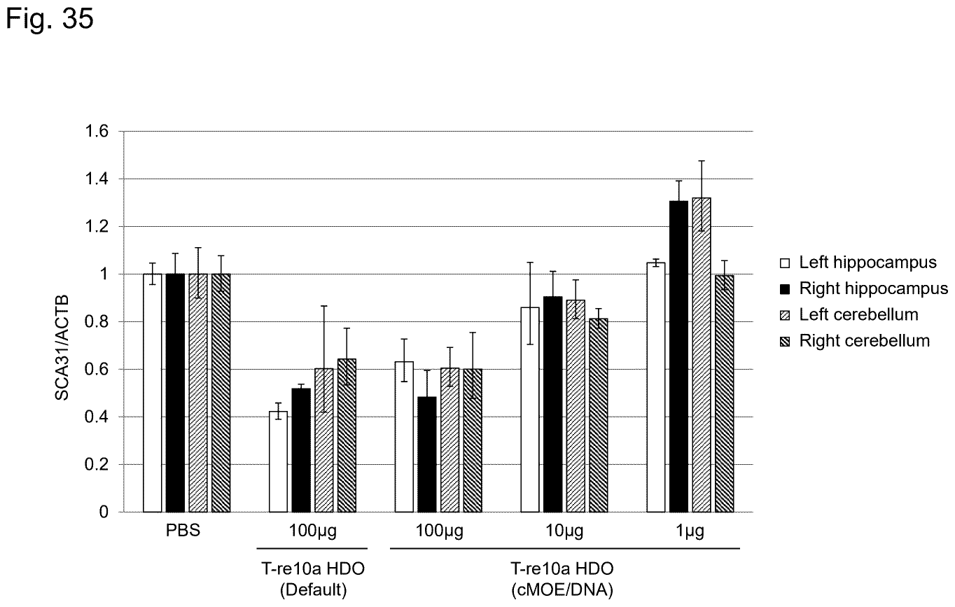

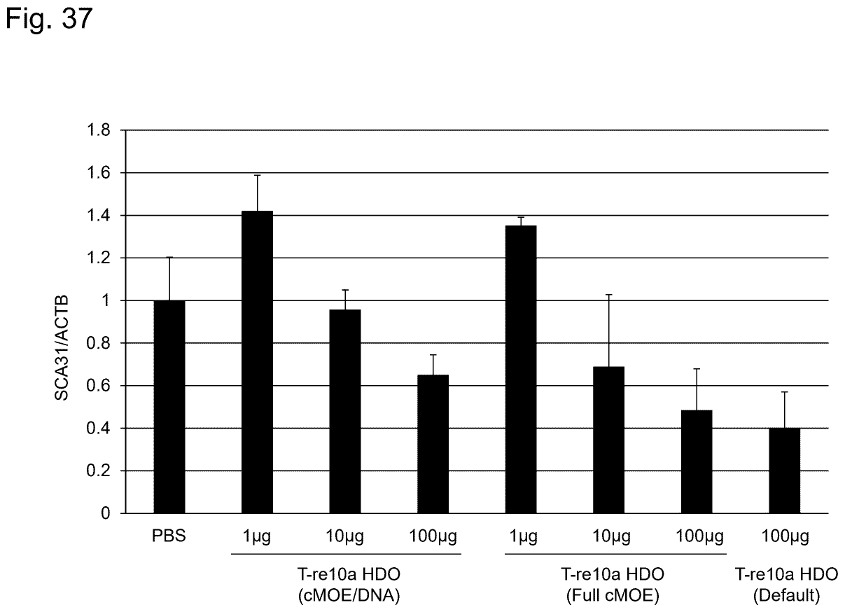

The results of reducing expression of SCA31 mutant repeats (TK2) by administration of 100 μg of T-re10a HDO (Default), administration of 100 μg, 10 μg, or 1 μg of T-re10a HDO (cMOE/DNA), and administration of 100 μg, 10 μg, or 1 μg of T-re10a HDO (Full cMOE) are shown in FIGS. 35 and 37. It was shown that the effects of reducing expression of the SCA31 mutant repeats (TK2) were increased by increasing the doses of T-re10a HDO (cMOE/DNA) and T-re10a HDO (Full cMOE).

All publications, patents, and patent applications cited herein are incorporated herein directly by reference.

1. A nucleic acid molecule having an antisense effect on the transcriptional product of a mutant gene comprising an abnormal repeat region, wherein:
said abnormal repeat region consists of a 5′ region, a core repeat region, and a 3′ region,
said core repeat region consists of a base sequence consisting of multiple repeats of the base sequence TGGAA or a base sequence complementary thereto, and
said nucleic acid molecule comprises a base sequence complementary to four or more consecutive bases in said abnormal repeat region in said transcriptional product, and comprises a target binding region that is capable of hybridizing to said transcriptional product.

2. The nucleic acid molecule of claim 1, wherein said mutant gene is a mutant form of the Brain Expressed Associated With NEDD4 1 (BEAN1) gene, said core repeat region consists of a base sequence consisting of multiple repeats of the base sequence TGGAA, and said 5′ region comprises the base sequence TCAC.

3. The nucleic acid molecule of claim 2, wherein said 5′ region consists of a base sequence selected from the group consisting of the following (a1) to (a4):
(a1) the base sequence of SEQ ID NO: 3;
(a2) the base sequence of SEQ ID NO: 4;
(a3) the base sequence TCAC; and
(a4) a base sequence formed by linking the base sequence TCAC and a base sequence consisting of multiple repeats of the base sequence TAGAA from 5′ side in this order.

4. The nucleic acid molecule of claim 1, wherein said mutant gene is a mutant form of the Brain Expressed Associated With NEDD4 1 (BEAN1) gene, said core repeat region consists of a base sequence consisting of multiple repeats of the base sequence TGGAA, and said 3′ region comprises a sequence consisting of multiple repeats of the base sequence TAGAA and a sequence consisting of multiple repeats of the base sequence of SEQ ID NO: 6.

5. The nucleic acid molecule of claim 4, wherein said 3′ region consists of a base sequence selected from the group consisting of the following (b1) to (b5):
(b1) a base sequence formed by linking the base sequence of SEQ ID NO: 7, a base sequence consisting of multiple repeats of the base sequence TAGAA, and a base sequence consisting of multiple repeats of the base sequence of SEQ ID NO: 6, from 5′ side in this order;
(b2) a base sequence formed by linking the base sequence of SEQ ID NO: 9, a base sequence consisting of multiple repeats of the base sequence TAGAA, and a base sequence consisting of multiple repeats of the base sequence of SEQ ID NO: 6, from 5′ side in this order;
(b3) a base sequence formed by linking the base sequence of SEQ ID NO: 11, a base sequence consisting of multiple repeats of the base sequence TAGAA, and a base sequence consisting of multiple repeats of the base sequence of SEQ ID NO: 6, from 5′ side in this order;
(b4) a base sequence formed by linking the base sequence of SEQ ID NO: 12, a base sequence consisting of multiple repeats of the base sequence TAGAA, a base sequence consisting of multiple repeats of the base sequence of SEQ ID NO: 6, and a base sequence consisting of multiple repeats of the base sequence of SEQ ID NO: 13, from 5′ side in this order; and
(b5) a base sequence formed by linking the base sequence of SEQ ID NO: 14, a base sequence consisting of multiple repeats of the base sequence TAGAA, and a base sequence consisting of multiple repeats of the base sequence of SEQ ID NO: 6, from 5′ side in this order.

6.-8. (canceled)

9. The nucleic acid molecule of claim 2, which consists of a base sequence selected from the group consisting of SEQ ID NOs: 156 to 189 and 207 to 209, provided that “t” in the sequence may be “u”.

10. The nucleic acid molecule of claim 1, wherein said mutant gene is a mutant form of the Thymidine Kinase 2 (TK2) gene, said core repeat region consists of the base sequence which is complementary to a base sequence consisting of multiple repeats of the base sequence TGGAA, and said 5′ region comprises a sequence consisting of multiple repeats of the base sequence of SEQ ID NO: 16 and a sequence consisting of multiple repeats of the base sequence TTCTA.

11. The nucleic acid molecule of claim 10, wherein said 5′ region consists of any base sequence selected from the group consisting of the following (c1) to (c5):
(c1) a base sequence formed by linking a base sequence consisting of multiple repeats of the base sequence of SEQ ID NO: 16, a base sequence consisting of multiple repeats of the base sequence TTCTA, and the base sequence of SEQ ID NO: 19 from 5′ side in this order;
(c2) a base sequence formed by linking a base sequence consisting of multiple repeats of the base sequence of SEQ ID NO: 16, a base sequence consisting of multiple repeats of the base sequence TTCTA, and the base sequence of SEQ ID NO: 21 from 5′ side in this order;
(c3) a base sequence formed by linking a base sequence consisting of multiple repeats of the base sequence of SEQ ID NO: 16, a base sequence consisting of multiple repeats of the base sequence TTCTA, and the base sequence of SEQ ID NO: 22 from 5′ side in this order;
(c4) a base sequence formed by linking the base sequence of SEQ ID NO: 23, a base sequence consisting of multiple repeats of the base sequence of SEQ ID NO: 16, a base sequence consisting of multiple repeats of the base sequence TTCTA, and the base sequence of SEQ ID NO: 24 from 5′ side in this order; and
(c5) a base sequence formed by linking a base sequence consisting of multiple repeats of the base sequence of SEQ ID NO: 16, a base sequence consisting of multiple repeats of the base sequence TTCTA, and the base sequence of SEQ ID NO: 29 from 5′ side in this order.

12. The nucleic acid molecule of claim 1, wherein said mutant gene is a mutant form of the Thymidine Kinase 2 (TK2) gene, said core repeat region consists of the base sequence which is complementary to a base sequence consisting of multiple repeats of the base sequence TGGAA, and said 3′ region comprises the base sequence GTGA.

13. The nucleic acid molecule of claim 12, wherein said 3′ region consists of a base sequence selected from the group consisting of the following (d1) to (d4):
(d1) the base sequence of SEQ ID NO: 27;
(d2) the base sequence of SEQ ID NO: 28;
(d3) the base sequence GTGA; and
(d4) the base sequence formed by linking a base sequence consisting of multiple repeats of the base sequence TTCTA and the base sequence GTGA from 5′ side in this order.

14.-16. (canceled)

17. The nucleic acid molecule of claim 10, which consists of a base sequence selected from the group consisting of SEQ ID NOs: 33 to 53, 58 to 139, 141 to 150, 152 to 155, and 204 to 206, provided that “t” in the sequence may be “u”.

18. The nucleic acid molecule of claim 1, wherein said mutant gene is a mutant form of the Ataxin 10 (ATXN10) gene, said core repeat region consists of the base sequence which is complementary to a base sequence consisting of multiple repeats of the base sequence TGGAA, and the full-length of said target binding region is comprised in the base sequence complementary to said core repeat region.

19. The nucleic acid molecule of claim 18, which consists of a base sequence selected from the group consisting of SEQ ID NOs: 33 to 37, 58 to 61, 64 to 67, 70 to 139, 141 to 150, 152 to 155, and 204 to 206, provided that “t” in the sequence may be “u”.

20.-21. (canceled)

22. The nucleic acid molecule of claim 1, which is a gapmer.

23.-32. (canceled)

33. A double-stranded nucleic acid complex comprising a first nucleic acid strand consisting of the nucleic acid molecule of claim 1 and a second nucleic acid strand comprising a base sequence complementary to said first nucleic acid strand.

34.-41. (canceled)

42. The double-stranded nucleic acid complex of claim 33, wherein said second nucleic acid strand comprises one or more 2′-O-methoxyethyl-modified nucleosides.

43. The double-stranded nucleic acid complex of claim 42, wherein at least 20% of the total number of nucleosides in said second nucleic acid strand are 2′-O-methoxyethyl-modified nucleosides.

44.-48. (canceled)

49. The double-stranded nucleic acid complex of claim 33, wherein said second nucleic acid strand is bound to tocopherol or cholesterol or an analog thereof.

50. The double-stranded nucleic acid complex of claim 33, wherein:
the base sequence of said first nucleic acid strand consists of the base sequence of SEQ ID NO: 65, provided that “t” in the sequence may be “u”, and
the base sequence of said second nucleic acid strand consists of any a base sequence selected from the group consisting of SEQ ID NO: 140, SEQ ID NO: 200, SEQ ID NO: 201, SEQ ID NO: 202, and SEQ ID NO: 203, provided that “t” in the sequence may be “u”.

51. The double-stranded nucleic acid complex of claim 50, wherein:
said first nucleic acid strand consists of the nucleic acid strand of SEQ ID NO: 65, and
said second nucleic acid strand consists of a nucleic acid strand selected from the group consisting of SEQ ID NO: 140, SEQ ID NO: 200, SEQ ID NO: 201, SEQ ID NO: 202, and SEQ ID NO: 203.

52.-61. (canceled)


SEQUENCE LISTING (209 sequences)

SEQ ID NO: 1-2 - skipped (2 entries)

SEQ ID NO: 3 - DNA, 19 bp, Homo sapiens
  source 1..19: mol_type = genomic DNA
tcactaaaatagaatagaa

SEQ ID NO: 4 - DNA, 29 bp, Homo sapiens
  source 1..29: mol_type = genomic DNA
tcactaaaatagaatagaatagaatagaa

SEQ ID NO: 5 - skipped

SEQ ID NO: 6 - DNA, 10 bp, Homo sapiens
  source 1..10: mol_type = genomic DNA
taaaatagaa

SEQ ID NO: 7 - DNA, 52 bp, Homo sapiens
  source 1..52: mol_type = genomic DNA
tgggaatggaatgggaatagaatagaatggaatggaatagaatagaatggaa

SEQ ID NO: 8 - skipped

SEQ ID NO: 9 - DNA, 52 bp, Homo sapiens
  source 1..52: mol_type = genomic DNA
tgggaatagaatgggaatagaatagaatggaatggaatggaatagaatggaa

SEQ ID NO: 10 - DNA, 22 bp, synthetic construct
  misc_feature 1..22: note = primer
  source 1..22: mol_type = other DNA
tggatagaaaccccagcttcct

SEQ ID NO: 11 - DNA, 117 bp, Homo sapiens
  source 1..117: mol_type = genomic DNA
tgggaatagaatgggaatggaatggaatggaatagaatggaatagaatggaatagaatggaatagaatggaatagaatagaatagaatagaatagaatagaatggaatagaatggaa

SEQ ID NO: 12 - DNA, 30 bp, Homo sapiens
  source 1..30: mol_type = genomic DNA
tagaatagaatggaatagaatagaatggaa

SEQ ID NO: 13 - DNA, 20 bp, Homo sapiens
  source 1..20: mol_type = genomic DNA
taaaataaaataaaatagaa

SEQ ID NO: 14 - DNA, 52 bp, Homo sapiens; identical to SEQ ID NO: 9
  source 1..52: mol_type = genomic DNA

SEQ ID NO: 15 - DNA, 22 bp, synthetic construct
  misc_feature 1..22: note = primer
  source 1..22: mol_type = other DNA
ctgcctccgacacttttcaatt

SEQ ID NO: 16 - DNA, 10 bp, Homo sapiens
  source 1..10: mol_type = genomic DNA
ttctatttta

SEQ ID NO: 17 - skipped

SEQ ID NO: 18 - DNA, 24 bp, synthetic construct
  misc_feature 1..24: note = primer
  source 1..24: mol_type = other DNA
tggctgcacatagctttatctctt

SEQ ID NO: 19 - DNA, 52 bp, Homo sapiens
  source 1..52: mol_type = genomic DNA
ttccattctattctattccattccattctattctattcccattccattccca

SEQ ID NO: 20 - DNA, 20 bp, synthetic construct
  misc_feature 1..20: note = primer
  source 1..20: mol_type = other DNA
aagcccaatctggaagcaaa

SEQ ID NO: 21 - DNA, 52 bp, Homo sapiens
  source 1..52: mol_type = genomic DNA
ttccattctattccattccattccattctattctattcccattctattccca

SEQ ID NO: 22 - DNA, 117 bp, Homo sapiens
  source 1..117: mol_type = genomic DNA
ttccattctattccattctattctattctattctattctattctattccattctattccattctattccattctattccattctattccattccattccattcccattctattccca

SEQ ID NO: 23 - DNA, 20 bp, Homo sapiens
  source 1..20: mol_type = genomic DNA
ttctattttattttatttta

SEQ ID NO: 24 - DNA, 30 bp, Homo sapiens
  source 1..30: mol_type = genomic DNA
ttccattctattctattccattctattcta

SEQ ID NO: 25 - DNA, 20 bp, synthetic construct
  misc_feature 1..20: note = primer
  source 1..20: mol_type = other DNA
cagccactgcccaggactaa

SEQ ID NO: 26 - skipped

SEQ ID NO: 27 - DNA, 19 bp, Homo sapiens
  source 1..19: mol_type = genomic DNA
ttctattctattttagtga

SEQ ID NO: 28 - DNA, 29 bp, Homo sapiens
  source 1..29: mol_type = genomic DNA
ttctattctattctattctattttagtga

SEQ ID NO: 29 - DNA, 52 bp, Homo sapiens; identical to SEQ ID NO: 21
  source 1..52: mol_type = genomic DNA

SEQ ID NO: 30 - DNA, 21 bp, synthetic construct
  misc_feature 1..21: note = primer
  source 1..21: mol_type = other DNA
gccacttctctgcctcagaga

SEQ ID NO: 31 - DNA, 25 bp, synthetic construct
  misc_feature 1..25: note = probe
  source 1..25: mol_type = other DNA
ttccattccattccattccattcca

SEQ ID NO: 32 - DNA, 25 bp, synthetic construct
  misc_feature 1..25: note = probe
  source 1..25: mol_type = other DNA
tggaatggaatggaatggaatggaa

SEQ ID NO: 33 - DNA, 13 bp, synthetic construct
  modified_base order(1..2,11..13): mod_base = OTHER; note = locked nucleic acid
  modified_base 1..13: mod_base = OTHER; note = phosphorothioate bond
  source 1..13: mol_type = other DNA
ggaatggaatgga

SEQ ID NO: 34 - DNA, 13 bp, synthetic construct
  modified_base order(1..2,11..13): mod_base = OTHER; note = locked nucleic acid
  modified_base 1..13: mod_base = OTHER; note = phosphorothioate bond
  source 1..13: mol_type = other DNA
tggaatggaatgg

SEQ ID NO: 35 - DNA, 13 bp, synthetic construct
  modified_base order(1..2,11..13): mod_base = OTHER; note = locked nucleic acid
  modified_base 1..13: mod_base = OTHER; note = phosphorothioate bond
  source 1..13: mol_type = other DNA
aatggaatggaat

SEQ ID NO: 36 - DNA, 13 bp, synthetic construct
  modified_base order(1..2,11..13): mod_base = OTHER; note = locked nucleic acid
  modified_base 1..13: mod_base = OTHER; note = phosphorothioate bond
  source 1..13: mol_type = other DNA
atggaatggaatg

SEQ ID NO: 37 - DNA, 13 bp, synthetic construct
  modified_base order(1..2,11..13): mod_base = OTHER; note = locked nucleic acid
  modified_base 1..13: mod_base = OTHER; note = phosphorothioate bond
  source 1..13: mol_type = other DNA
gaatggaatggaa

SEQ ID NO: 38 - DNA, 13 bp, synthetic construct
  modified_base order(1..2,11..13): mod_base = OTHER; note = locked nucleic acid
  modified_base 1..13: mod_base = OTHER; note = phosphorothioate bond
  source 1..13: mol_type = other DNA
tagaatggaatgg

SEQ ID NO: 39 - DNA, 13 bp, synthetic construct
  modified_base order(1..2,11..13): mod_base = OTHER; note = locked nucleic acid
  modified_base 1..13: mod_base = OTHER; note = phosphorothioate bond
  source 1..13: mol_type = other DNA
agaatggaatgga

SEQ ID NO: 40 - DNA, 16 bp, synthetic construct
  modified_base order(1..3,14..16): mod_base = OTHER; note = locked nucleic acid
  modified_base 1..16: mod_base = OTHER; note = phosphorothioate bond
  source 1..16: mol_type = other DNA
agaatagaatggaatg

SEQ ID NO: 41 - DNA, 16 bp, synthetic construct
  modified_base order(1..3,14..16): mod_base = OTHER; note = locked nucleic acid
  modified_base 1..16: mod_base = OTHER; note = phosphorothioate bond
  source 1..16: mol_type = other DNA
gaatagaatggaatgg

SEQ ID NO: 42 - DNA, 16 bp, synthetic construct
  modified_base order(1..3,14..16): mod_base = OTHER; note = locked nucleic acid
  modified_base 1..16: mod_base = OTHER; note = phosphorothioate bond
  source 1..16: mol_type = other DNA
aatagaatggaatgga

SEQ ID NO: 43 - DNA, 16 bp, synthetic construct
  modified_base order(1..3,14..16): mod_base = OTHER; note = locked nucleic acid
  modified_base 1..16: mod_base = OTHER; note = phosphorothioate bond
  source 1..16: mol_type = other DNA
atagaatggaatggaa

SEQ ID NO: 44 - DNA, 16 bp, synthetic construct
  modified_base order(1..3,14..16): mod_base = OTHER; note = locked nucleic acid
  modified_base 1..16: mod_base = OTHER; note = phosphorothioate bond
  source 1..16: mol_type = other DNA
tagaatggaatggaat

SEQ ID NO: 45 - DNA, 16 bp, synthetic construct
  modified_base order(1..3,14..16): mod_base = OTHER; note = locked nucleic acid
  modified_base 1..16: mod_base = OTHER; note = phosphorothioate bond
  source 1..16: mol_type = other DNA
agaatggaatggaatg

SEQ ID NO: 46 - DNA, 14 bp, synthetic construct
  modified_base order(1..3,12..14): mod_base = OTHER; note = locked nucleic acid
  modified_base 1..14: mod_base = OTHER; note = phosphorothioate bond
  source 1..14: mol_type = other DNA
tggaatggaatggg

SEQ ID NO: 47 - DNA, 14 bp, synthetic construct
  modified_base order(1..3,12..14): mod_base = OTHER; note = locked nucleic acid
  modified_base 1..14: mod_base = OTHER; note = phosphorothioate bond
  source 1..14: mol_type = other DNA
gaatggaatgggaa

SEQ ID NO: 48 - DNA, 14 bp, synthetic construct
  modified_base order(1..3,12..14): mod_base = OTHER; note = locked nucleic acid
  modified_base 1..14: mod_base = OTHER; note = phosphorothioate bond
  source 1..14: mol_type = other DNA
atggaatgggaatg

SEQ ID NO: 49 - DNA, 14 bp, synthetic construct
  modified_base order(1..3,12..14): mod_base = OTHER; note = locked nucleic acid
  modified_base 1..14: mod_base = OTHER; note = phosphorothioate bond
  source 1..14: mol_type = other DNA
ggaatgggaatgga

SEQ ID NO: 50 - DNA, 14 bp, synthetic construct
  modified_base order(1..3,12..14): mod_base = OTHER; note = locked nucleic acid
  modified_base 1..14: mod_base = OTHER; note = phosphorothioate bond
  source 1..14: mol_type = other DNA
ggaatggaatggga

SEQ ID NO: 51 - DNA, 14 bp, synthetic construct
  modified_base order(1..3,12..14): mod_base = OTHER; note = locked nucleic acid
  modified_base 1..14: mod_base = OTHER; note = phosphorothioate bond
  source 1..14: mol_type = other DNA
tttcaatcactaaa

SEQ ID NO: 52 - DNA, 14 bp, synthetic construct
  modified_base order(1..3,12..14): mod_base = OTHER; note = locked nucleic acid
  modified_base 1..14: mod_base = OTHER; note = phosphorothioate bond
  modified_base 2: mod_base = OTHER; note = 5-methylcytosine
  source 1..14: mol_type = other DNA
tcaatcactaaaat

SEQ ID NO: 53 - DNA, 14 bp, synthetic construct
  modified_base order(1..3,12..14): mod_base = OTHER; note = locked nucleic acid
  modified_base 1..14: mod_base = OTHER; note = phosphorothioate bond
  source 1..14: mol_type = other DNA
aatcactaaaatag

SEQ ID NO: 54 - DNA, 13 bp, synthetic construct
  modified_base order(1..2,11..13): mod_base = OTHER; note = locked nucleic acid
  modified_base 1..13: mod_base = OTHER; note = phosphorothioate bond
  modified_base order(2,12): mod_base = OTHER; note = 5-methylcytosine
  source 1..13: mol_type = other DNA
gcattggtattca

SEQ ID NO: 55 - DNA, 13 bp, synthetic construct
  modified_base order(1..2,11..13): mod_base = OTHER; note = locked nucleic acid
  modified_base 1..13: mod_base = OTHER; note = phosphorothioate bond
  modified_base order(1,13): mod_base = OTHER; note = 5-methylcytosine
  source 1..13: mol_type = other DNA
cgtagttgtaatc

SEQ ID NO: 56 - DNA, 16 bp, synthetic construct
  modified_base order(1..3,14..16): mod_base = OTHER; note = locked nucleic acid
  modified_base 1..16: mod_base = OTHER; note = phosphorothioate bond
  modified_base order(1,16): mod_base = OTHER; note = 5-methylcytosine
  source 1..16: mol_type = other DNA
ctagttcactgaatgc

SEQ ID NO: 57 - DNA, 13 bp, synthetic construct
  modified_base order(1..2,11..13): mod_base = OTHER; note = locked nucleic acid
  modified_base 1..13: mod_base = OTHER; note = phosphorothioate bond
  modified_base order(1..2,12): mod_base = OTHER; note = 5-methylcytosine
  source 1..13: mol_type = other DNA
ccaacatttgtca

SEQ ID NO: 58 - DNA, 13 bp, synthetic construct
  modified_base order(1..2,11..13): mod_base = OTHER; note = 2'-O,4'-C-ethylene-bridged nucleic acid
  modified_base 1..13: mod_base = OTHER; note = phosphorothioate bond
  source 1..13: mol_type = other DNA
ggaatggaatgga

SEQ ID NO: 59 - DNA, 13 bp, synthetic construct
  modified_base order(1..2,11..13): mod_base = OTHER; note = 2'-O,4'-C-ethylene-bridged nucleic acid
  modified_base 1..13: mod_base = OTHER; note = phosphorothioate bond
  source 1..13: mol_type = other DNA
tggaatggaatgg

SEQ ID NO: 60 - DNA, 13 bp, synthetic construct
  modified_base order(1..2,11..13): mod_base = OTHER; note = 2'-O,4'-C-ethylene-bridged nucleic acid
  modified_base 1..13: mod_base = OTHER; note = phosphorothioate bond
  source 1..13: mol_type = other DNA
gaatggaatggaa

SEQ ID NO: 61 - DNA, 13 bp, synthetic construct
  modified_base order(1..2,11..13): mod_base = OTHER; note = 2'-O,4'-C-ethylene-bridged nucleic acid
  modified_base 1..13: mod_base = OTHER; note = phosphorothioate bond
  source 1..13: mol_type = other DNA
atggaatggaatg

SEQ ID NO: 62 - DNA, 13 bp, synthetic construct
  modified_base order(1..2,11..13): mod_base = OTHER; note = 2'-O,4'-C-ethylene-bridged nucleic acid
  modified_base 1..13: mod_base = OTHER; note = phosphorothioate bond
  source 1..13: mol_type = other DNA
agaatggaatgga

SEQ ID NO: 63 - DNA, 16 bp, synthetic construct
  modified_base order(1..3,14..16): mod_base = OTHER; note = 2'-O,4'-C-ethylene-bridged nucleic acid
  modified_base 1..16: mod_base = OTHER; note = phosphorothioate bond
  source 1..16: mol_type = other DNA
agaatggaatggaatg

SEQ ID NO: 64 - DNA, 13 bp, synthetic construct
  modified_base order(1,3,11,13): mod_base = OTHER; note = 2'-O,4'-C-ethylene-bridged nucleic acid
  modified_base 1..13: mod_base = OTHER; note = phosphorothioate bond
  modified_base order(2,12): mod_base = OTHER; note = 2'-O-methyl RNA
  source 1..13: mol_type = other DNA
ggaatggaatgga

SEQ ID NO: 65 - DNA, 13 bp, synthetic construct
  modified_base order(1,3,11,13): mod_base = OTHER; note = 2'-O,4'-C-ethylene-bridged nucleic acid
  modified_base 1..13: mod_base = OTHER; note = phosphorothioate bond
  modified_base order(2,12): mod_base = OTHER; note = 2'-O-methyl RNA
  source 1..13: mol_type = other DNA
tggaatggaatgg

SEQ ID NO: 66 - DNA, 13 bp, synthetic construct
  modified_base order(1,3,11,13): mod_base = OTHER; note = 2'-O,4'-C-ethylene-bridged nucleic acid
  modified_base 1..13: mod_base = OTHER; note = phosphorothioate bond
  modified_base order(2,12): mod_base = OTHER; note = 2'-O-methyl RNA
  source 1..13: mol_type = other DNA
gaatggaatggaa

SEQ ID NO: 67 - DNA, 13 bp, synthetic construct
  modified_base order(1,3,11,13): mod_base = OTHER; note = 2'-O,4'-C-ethylene-bridged nucleic acid
  modified_base 1..13: mod_base = OTHER; note = phosphorothioate bond
  modified_base order(2,12): mod_base = OTHER; note = 2'-O-methyl RNA
  modified_base order(2,12): mod_base = OTHER; note = uracil
  source 1..13: mol_type = other DNA
atggaatggaatg

SEQ ID NO: 68 - DNA, 13 bp, synthetic construct
  modified_base order(1,3,11,13): mod_base = OTHER; note = 2'-O,4'-C-ethylene-bridged nucleic acid
  modified_base 1..13: mod_base = OTHER; note = phosphorothioate bond
  modified_base order(2,12): mod_base = OTHER; note = 2'-O-methyl RNA
  source 1..13: mol_type = other DNA
agaatggaatgga

SEQ ID NO: 69 - DNA, 16 bp, synthetic construct
  modified_base order(1,3,14,16): mod_base = OTHER; note = 2'-O,4'-C-ethylene-bridged nucleic acid
  modified_base 1..16: mod_base = OTHER; note = phosphorothioate bond
  modified_base order(2,15): mod_base = OTHER; note = 2'-O-methyl RNA
  modified_base 15: mod_base = OTHER; note = uracil
  source 1..16: mol_type = other DNA
agaatggaatggaatg

SEQ ID NO: 70 - DNA, 13 bp, synthetic construct
  modified_base order(1,3,11): mod_base = OTHER; note = 2'-O,4'-C-ethylene-bridged nucleic acid
  modified_base 1..13: mod_base = OTHER; note = phosphorothioate bond
  modified_base order(2,12..13): mod_base = OTHER; note = 2'-O-methyl RNA
  source 1..13: mol_type = other DNA
tggaatggaatgg

SEQ ID NO: 71 - DNA, 13 bp, synthetic construct
  modified_base order(1,11,13): mod_base = OTHER; note = 2'-O,4'-C-ethylene-bridged nucleic acid
  modified_base 1..13: mod_base = OTHER; note = phosphorothioate bond
  modified_base order(2..3,12): mod_base = OTHER; note = 2'-O-methyl RNA
  source 1..13: mol_type = other DNA
tggaatggaatgg

SEQ ID NO: 72 - DNA, 13 bp, synthetic construct
  modified_base order(1,11): mod_base = OTHER; note = 2'-O,4'-C-ethylene-bridged nucleic acid
  modified_base order(2..3,12..13): mod_base = OTHER; note = 2'-O-methyl RNA
  modified_base 1..13: mod_base = OTHER; note = phosphorothioate bond
  source 1..13: mol_type = other DNA
tggaatggaatgg

SEQ ID NO: 73 - DNA, 13 bp, synthetic construct
  modified_base order(2,12): mod_base = OTHER; note = 2'-O,4'-C-ethylene-bridged nucleic acid
  modified_base order(1,3,11,13): mod_base = OTHER; note = 2'-O-methyl RNA
  modified_base 1..13: mod_base = OTHER; note = phosphorothioate bond
  modified_base order(1,11): mod_base = OTHER; note = uracil
  source 1..13: mol_type = other DNA
tggaatggaatgg

SEQ ID NO: 74 - DNA, 14 bp, synthetic construct
  modified_base order(2,4,12,14): mod_base = OTHER; note = 2'-O,4'-C-ethylene-bridged nucleic acid
  modified_base order(1,3,13): mod_base = OTHER; note = 2'-O-methyl RNA
  modified_base 1..14: mod_base = OTHER; note = phosphorothioate bond
  source 1..14: mol_type = other DNA
atggaatggaatgg

SEQ ID NO: 75 - DNA, 14 bp, synthetic construct
  modified_base order(1,3,11,13): mod_base = OTHER; note = 2'-O,4'-C-ethylene-bridged nucleic acid
  modified_base order(2,12,14): mod_base = OTHER; note = 2'-O-methyl RNA
  modified_base 1..14: mod_base = OTHER; note = phosphorothioate bond
  source 1..14: mol_type = other DNA
tggaatggaatgga

SEQ ID NO: 76 - DNA, 15 bp, synthetic construct
  modified_base order(2,4,12,14): mod_base = OTHER; note = 2'-O,4'-C-ethylene-bridged nucleic acid
  modified_base order(1,3,13,15): mod_base = OTHER; note = 2'-O-methyl RNA
  modified_base 1..15: mod_base = OTHER; note = phosphorothioate bond
  source 1..15: mol_type = other DNA
atggaatggaatgga

SEQ ID NO: 77 - DNA, 14 bp, synthetic construct
  modified_base order(1..2,4,12,14): mod_base = OTHER; note = 2'-O,4'-C-ethylene-bridged nucleic acid
  modified_base order(3,13): mod_base = OTHER; note = 2'-O-methyl RNA
  modified_base 1..14: mod_base = OTHER; note = phosphorothioate bond
  source 1..14: mol_type = other DNA
atggaatggaatgg

SEQ ID NO: 78 - DNA, 14 bp, synthetic construct
  modified_base order(1,3,11,13..14): mod_base = OTHER; note = 2'-O,4'-C-ethylene-bridged nucleic acid
  modified_base order(2,12): mod_base = OTHER; note = 2'-O-methyl RNA
  modified_base 1..14: mod_base = OTHER; note = phosphorothioate bond
  source 1..14: mol_type = other DNA
tggaatggaatgga

SEQ ID NO: 79 - DNA, 15 bp, synthetic construct
  modified_base order(1..2,4,12,14..15): mod_base = OTHER; note = 2'-O,4'-C-ethylene-bridged nucleic acid
  modified_base order(3,13): mod_base = OTHER; note = 2'-O-methyl RNA
  modified_base 1..15: mod_base = OTHER; note = phosphorothioate bond
  source 1..15: mol_type = other DNA
atggaatggaatgga

SEQ ID NO: 80 - DNA, 15 bp, synthetic construct
  modified_base order(1..2,12,15): mod_base = OTHER; note = 2'-O,4'-C-ethylene-bridged nucleic acid
  modified_base order(3..4,13..14): mod_base = OTHER; note = 2'-O-methyl RNA
  modified_base 1..15: mod_base = OTHER; note = phosphorothioate bond
  source 1..15: mol_type = other DNA
atggaatggaatgga

SEQ ID NO: 81 - DNA, 15 bp, synthetic construct
  modified_base order(1..2,14..15): mod_base = OTHER; note = 2'-O-methyl RNA
  source 1..15: mol_type = other DNA
  modified_base order(3..4,12..13): mod_base = OTHER; note = 2'-O,4'-C-ethylene-bridged nucleic acid
  modified_base 2: mod_base = OTHER; note = uracil
atggaatggaatgga

SEQ ID NO: 82 - DNA, 16 bp, synthetic construct
  modified_base order(1,3..4,14,16): mod_base = OTHER; note = 2'-O,4'-C-ethylene-bridged nucleic acid
  modified_base order(2,15): mod_base = OTHER; note = 2'-O-methyl RNA
  modified_base 1..16: mod_base = OTHER; note = phosphorothioate bond
  modified_base 15: mod_base = OTHER; note = uracil
  source 1..16: mol_type = other DNA
ggaatggaatggaatg

SEQ ID NO: 83 - DNA, 16 bp, synthetic construct
  modified_base order(1,3,14,16): mod_base = OTHER; note = 2'-O,4'-C-ethylene-bridged nucleic acid
  modified_base order(2,15): mod_base = OTHER; note = 2'-O-methyl RNA
  modified_base 1..16: mod_base = OTHER; note = phosphorothioate bond
  source 1..16: mol_type = other DNA
gaatggaatggaatgg

SEQ ID NO: 84 - DNA, 16 bp, synthetic construct
  modified_base order(1,3,14,16): mod_base = OTHER; note = 2'-O,4'-C-ethylene-bridged nucleic acid
  modified_base order(2,15): mod_base = OTHER; note = 2'-O-methyl RNA
  modified_base 1..16: mod_base = OTHER; note = phosphorothioate bond
  source 1..16: mol_type = other DNA
aatggaatggaatgga

SEQ ID NO: 85 - DNA, 16 bp, synthetic construct
  modified_base order(2,15): mod_base = OTHER; note = 2'-O-methyl RNA
  source 1..16: mol_type = other DNA
  modified_base order(1,3,14,16): mod_base = OTHER; note = 2'-O,4'-C-ethylene-bridged nucleic acid
  modified_base 2: mod_base = OTHER; note = uracil
atggaatggaatggaa

SEQ ID NO: 86 - DNA, 16 bp, synthetic construct
  modified_base order(1,3,14,16): mod_base = OTHER; note = 2'-O,4'-C-ethylene-bridged nucleic acid
  modified_base order(2,15): mod_base = OTHER; note = 2'-O-methyl RNA
  modified_base 1..16: mod_base = OTHER; note = phosphorothioate bond
  source 1..16: mol_type = other DNA
tggaatggaatggaat

SEQ ID NO: 87 - DNA, 20 bp, synthetic construct
  modified_base order(1..5,16..20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 88 - DNA, 20 bp, synthetic construct
  modified_base order(1..5,16..20): mod_base = OTHER; note = 2'-O-methoxyethyl RNA
  modified_base 1..20: mod_base = OTHER; note = phosphorothioate bond
  source 1..20: mol_type = other DNA
ggaatggaatggaatggaat

SEQ ID NO: 89 - DNA, 20 bp, synthetic construct
  modified_base order(1..5,16..20): mod_base = OTHER; note = 2'-O-methoxyethyl RNA
  modified_base 1..20: mod_base = OTHER; note = phosphorothioate bond
  source 1..20: mol_type = other DNA
gaatggaatggaatggaatg

SEQ ID NO: 90 - DNA, 20 bp, synthetic construct
  modified_base order(1..5,16..20): mod_base = OTHER; note = 2'-O-methoxyethyl RNA
  modified_base 1..20: mod_base = OTHER; note = phosphorothioate bond
  source 1..20: mol_type = other DNA
aatggaatggaatggaatgg

SEQ ID NO: 91 - DNA, 20 bp, synthetic construct
  modified_base order(1..5,16..20): mod_base = OTHER; note = 2'-O-methoxyethyl RNA
  modified_base 1..20: mod_base = OTHER; note = phosphorothioate bond
  source 1..20: mol_type = other DNA
atggaatggaatggaatgga

SEQ ID NO: 92 - DNA, 20 bp, synthetic construct
  modified_base order(1,20): mod_base = OTHER; note = 2'-O,4'-C-ethylene-bridged nucleic acid
  modified_base 1..20: mod_base = OTHER; note = phosphorothioate bond
  modified_base order(2..5,16..19): mod_base = OTHER; note = 2'-O-methoxyethyl RNA
  source 1..20: mol_type = other DNA
tggaatggaatggaatggaa

SEQ ID NO: 93 - DNA, 20 bp, synthetic construct
  modified_base order(1,19): mod_base = OTHER; note = 2'-O,4'-C-ethylene-bridged nucleic acid
  modified_base order(2..5,16..18,20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 94 - DNA, 20 bp, synthetic construct
  modified_base order(1,16): mod_base = OTHER; note = 2'-O,4'-C-ethylene-bridged nucleic acid
  modified_base order(2..5,17..20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 95 - DNA, 20 bp, synthetic construct
  modified_base order(4,20): mod_base = OTHER; note = 2'-O,4'-C-ethylene-bridged nucleic acid
  modified_base order(1..3,5,16..19): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 96 - DNA, 20 bp, synthetic construct
  modified_base order(4,19): mod_base = OTHER; note = 2'-O,4'-C-ethylene-bridged nucleic acid
  modified_base order(1..3,5,16..18,20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 97 - DNA, 20 bp, synthetic construct
  modified_base order(4,16): mod_base = OTHER; note = 2'-O,4'-C-ethylene-bridged nucleic acid
  modified_base order(1..3,5,17..20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 98 - DNA, 20 bp, synthetic construct
  modified_base order(5,19): mod_base = OTHER; note = 2'-O,4'-C-ethylene-bridged nucleic acid
  modified_base order(1..4,16..18,20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 99 - DNA, 20 bp, synthetic construct
  modified_base order(5,20): mod_base = OTHER; note = 2'-O,4'-C-ethylene-bridged nucleic acid
  modified_base order(1..4,16..19): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 100 - DNA, 20 bp, synthetic construct
  modified_base order(5,16): mod_base = OTHER; note = 2'-O,4'-C-ethylene-bridged nucleic acid
  modified_base order(1..4,17..20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 101 - DNA, 20 bp, synthetic construct
  modified_base order(1,4,16): mod_base = OTHER; note = 2'-O,4'-C-ethylene-bridged nucleic acid
  modified_base order(2..3,5,17..20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 102 - DNA, 20 bp, synthetic construct
  modified_base order(1,4,19): mod_base = OTHER; note = 2'-O,4'-C-ethylene-bridged nucleic acid
  modified_base order(2..3,5,16..18,20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 103 - DNA, 20 bp, synthetic construct
  modified_base order(1,4,20): mod_base = OTHER; note = 2'-O,4'-C-ethylene-bridged nucleic acid
  modified_base order(2..3,5,16..19): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 104 - DNA, 20 bp, synthetic construct
  modified_base order(1,5,16): mod_base = OTHER; note = 2'-O,4'-C-ethylene-bridged nucleic acid
  modified_base order(2..4,17..20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 105 - DNA, 20 bp, synthetic construct
  modified_base order(1,5,19): mod_base = OTHER; note = 2'-O,4'-C-ethylene-bridged nucleic acid
  modified_base order(2..4,16..18,20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 106 - DNA, 20 bp, synthetic construct
  modified_base order(1,5,20): mod_base = OTHER; note = 2'-O,4'-C-ethylene-bridged nucleic acid
  modified_base 1..20: mod_base = OTHER; note = phosphorothioate bond
  modified_base order(2..4,16..19): mod_base = OTHER; note = 2'-O-methoxyethyl RNA
  source 1..20: mol_type = other DNA
tggaatggaatggaatggaa

SEQ ID NO: 107 - DNA, 20 bp, synthetic construct
  modified_base order(1,16,19): mod_base = OTHER; note = 2'-O,4'-C-ethylene-bridged nucleic acid
  modified_base order(2..5,17..18,20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 108 - DNA, 20 bp, synthetic construct
  modified_base order(1,16,20): mod_base = OTHER; note = 2'-O,4'-C-ethylene-bridged nucleic acid
  modified_base order(2..5,17..19): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 109 - DNA, 20 bp, synthetic construct
  modified_base order(4,16,19): mod_base = OTHER; note = 2'-O,4'-C-ethylene-bridged nucleic acid
  modified_base order(1..3,5,17..18,20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 110 - DNA, 20 bp, synthetic construct
  modified_base order(4,16,20): mod_base = OTHER; note = 2'-O,4'-C-ethylene-bridged nucleic acid
  modified_base order(1..3,5,17..19): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 111 - DNA, 20 bp, synthetic construct
  modified_base order(5,16,19): mod_base = OTHER; note = 2'-O,4'-C-ethylene-bridged nucleic acid
  modified_base order(1..4,17..18,20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 112 - DNA, 20 bp, synthetic construct
  modified_base order(5,16,20): mod_base = OTHER; note = 2'-O,4'-C-ethylene-bridged nucleic acid
  modified_base order(1..4,17..19): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 113 - DNA, 20 bp, synthetic construct
  modified_base order(1,4,16,19): mod_base = OTHER; note = 2'-O,4'-C-ethylene-bridged nucleic acid
  modified_base order(2..3,5,17..18,20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 114 - DNA, 20 bp, synthetic construct
  modified_base order(1,4,16,20): mod_base = OTHER; note = 2'-O,4'-C-ethylene-bridged nucleic acid
  modified_base order(2..3,5,17..19): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 115 - DNA, 20 bp, synthetic construct
  modified_base order(1,5,16,19): mod_base = OTHER; note = 2'-O,4'-C-ethylene-bridged nucleic acid
  modified_base order(2..4,17..18,20): mod_base = OTHER; note = 2'-O-methoxyethyl RN
  modified_base 1..20: mod_base = OTHER; note = phosphorothioate bond
  source 1..20: mol_type = other DNA
tggaatggaatggaatggaa

SEQ ID NO: 116 - DNA, 20 bp, synthetic construct
  modified_base order(1,5,16,20): mod_base = OTHER; note = 2'-O,4'-C-ethylene-bridged nucleic acid
  modified_base order(2..4,17..19): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 117 - DNA, 20 bp, synthetic construct
  modified_base 4..5: mod_base = OTHER; note = 2'-O,4'-C-ethylene-bridged nucleic acid
  modified_base order(1..3,16..20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 118 - DNA, 20 bp, synthetic construct
  modified_base 16..17: mod_base = OTHER; note = 2'-O,4'-C-ethylene-bridged nucleic acid
  modified_base order(1..5,18..20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 119 - DNA, 20 bp, synthetic construct
  modified_base order(3,5): mod_base = OTHER; note = 2'-O,4'-C-ethylene-bridged nucleic acid
  modified_base order(1..2,4,16..20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 120 - DNA, 20 bp, synthetic construct
  modified_base order(16,18): mod_base = OTHER; note = 2'-O,4'-C-ethylene-bridged nucleic acid
  modified_base order(1..5,17,19..20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 121 - DNA, 20 bp, synthetic construct
  modified_base order(4..5,16..17): mod_base = OTHER; note = 2'-O,4'-C-ethylene-bridged nucleic acid
  modified_base order(1..3,18..20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 122 - DNA, 20 bp, synthetic construct
  modified_base order(3,5,16,18): mod_base = OTHER; note = 2'-O,4'-C-ethylene-bridged nucleic acid
  modified_base order(1..2,4,17,19..20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 123 - DNA, 20 bp, synthetic construct
  modified_base order(4..5,16,18): mod_base = OTHER; note = 2'-O,4'-C-ethylene-bridged nucleic acid
  modified_base order(1..3,17,19..20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 124 - DNA, 20 bp, synthetic construct
  modified_base order(3,5,16..17): mod_base = OTHER; note = 2'-O,4'-C-ethylene-bridged nucleic acid
  modified_base order(1..2,4,18..20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 125 - DNA, 20 bp, synthetic construct
  modified_base order(1,3,5,16,18): mod_base = OTHER; note = 2'-O,4'-C-ethylene-bridged nucleic acid
  modified_base order(2,4,17,19..20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 126 - DNA, 20 bp, synthetic construct
  modified_base order(3,5,16,18,20): mod_base = OTHER; note = 2'-O,4'-C-ethylene-bridged nucleic acid
  modified_base order(1..2,4,17,19): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 127 - DNA, 20 bp, synthetic construct
  modified_base order(1..2,4,16,19): mod_base = OTHER; note = 2'-O,4'-C-ethylene-bridged nucleic acid
  modified_base order(3,5,17..18,20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 128 - DNA, 20 bp, synthetic construct
  modified_base order(1,3..4,16,19): mod_base = OTHER; note = 2'-O,4'-C-ethylene-bridged nucleic acid
  modified_base order(2,5,17..18,20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 129 - DNA, 20 bp, synthetic construct
  modified_base order(1,4..5,16,19): mod_base = OTHER; note = 2'-O,4'-C-ethylene-bridged nucleic acid
  modified_base order(2..3,17..18,20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 130 - DNA, 20 bp, synthetic construct
  modified_base order(1,4,16..17,19): mod_base = OTHER; note = 2'-O,4'-C-ethylene-bridged nucleic acid
  modified_base order(2..3,5,18,20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 131 - DNA, 20 bp, synthetic construct
  modified_base order(1,4,16,18..19): mod_base = OTHER; note = 2'-O,4'-C-ethylene-bridged nucleic acid
  modified_base order(2..3,5,17,20): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 132 - DNA, 20 bp, synthetic construct
  modified_base order(1,4,16,19..20): mod_base = OTHER; note = 2'-O,4'-C-ethylene-bridged nucleic acid
  modified_base order(2..3,5,17..18): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 133 - DNA, 20 bp, synthetic construct
  modified_base 1..5: mod_base = OTHER; note = 2'-O,4'-C-ethylene-bridged nucleic acid
  modified_base 16..20: mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 134 - DNA, 20 bp, synthetic construct
  modified_base 1..5: mod_base = OTHER; note = 2'-O-methoxyethyl RNA
  modified_base 16..20: mod_base = OTHER; note = 2'-O,4'-C-ethylene-bridged nucleic acid
  modified_base 1..20: mod_base = OTHER; note = phosphorothioate bond
  source 1..20: mol_type = other DNA
tggaatggaatggaatggaa

SEQ ID NO: 135 - DNA, 20 bp, synthetic construct
  modified_base order(1..5,16..20): mod_base = OTHER; note = 2'-O-methyl RNA
  modified_base order(1,16): mod_base = OTHER; note = uracil
  modified_base 1..20: mod_base = OTHER; note = phosphorothioate bond
  source 1..20: mol_type = other DNA
tggaatggaatggaatggaa

SEQ ID NO: 136 - DNA, 20 bp, synthetic construct
  modified_base order(1..3,5,16..19): mod_base = OTHER; note = 2'-O-methyl RNA
  modified_base order(1,16): mod_base = OTHER; note = 2'-O-methyl RNA
  modified_base order(4,20): mod_base = OTHER; note = 2'-O,4'-C-ethylene-bridged nucleic acid
  modified_base 1..20: mod_base = OTHER; note = phosphorothioate bond
  source 1..20: mol_type = other DNA
  modified_base order(1,16): mod_base = OTHER; note = uracil
tggaatggaatggaatggaa

SEQ ID NO: 137 - DNA, 20 bp, synthetic construct
  modified_base order(1..4,16..19): mod_base = OTHER; note = 2'-O-methyl RNA
  modified_base order(1,16): mod_base = OTHER; note = uracil
  modified_base order(5,20): mod_base = OTHER; note = 2'-O,4'-C-ethylene-bridged nucleic acid
  modified_base 1..20: mod_base = OTHER; note = phosphorothioate bond
  source 1..20: mol_type = other DNA
tggaatggaatggaatggaa

SEQ ID NO: 138 - DNA, 20 bp, synthetic construct
  modified_base order(1,5,16): mod_base = OTHER; note = 2'-O,4'-C-ethylene-bridged nucleic acid
  modified_base order(2..4,17..20): mod_base = OTHER; note = 2'-O-methyl RNA
  modified_base 1..20: mod_base = OTHER; note = phosphorothioate bond
  source 1..20: mol_type = other DNA
tggaatggaatggaatggaa

SEQ ID NO: 139 - DNA, 20 bp, synthetic construct
  modified_base order(1,4,16,19): mod_base = OTHER; note = 2'-O,4'-C-ethylene-bridged nucleic acid
  modified_base order(2..3,5,17..18,20): mod_base = OTHER; note = 2'-O-methyl RNA
  modified_base 1..20: mod_base = OTHER; note = phosphorothioate bond
  source 1..20: mol_type = other DNA
tggaatggaatggaatggaa

SEQ ID NO: 140 - RNA, 13 nt, synthetic construct
  modified_base order(1..3,12..13): mod_base = OTHER; note = 2'-O-methyl RNA
  modified_base order(1..4,11..13): mod_base = OTHER; note = phosphorothioate bond
  source 1..13: mol_type = other RNA
ccattccattcca

SEQ ID NO: 141 - DNA, 20 bp, synthetic construct
  modified_base order(1,4,16,20): mod_base = OTHER; note = 2'-O,4'-C-ethylene-bridged nucleic acid
  modified_base order(2..3,5,17..19): mod_base = OTHER; note = 2'-O-methoxyethyl RNA
  modified_base 1..20: mod_base = OTHER; note = phosphorothioate bond
  source 1..20: mol_type = other DNA
tggaatggaatggaatggaa

SEQ ID NO: 142 - DNA, 20 bp, synthetic construct
  modified_base order(1,4,16,20): mod_base = OTHER; note = 2'-O,4'-C-ethylene-bridged nucleic acid
  modified_base order(2..3,5,17..19): mod_base = OTHER; note = 2'-O-methoxyethyl RNA
  modified_base order(1..6,7..9,11..12,14..20): mod_base = OTHER; note = phosphorothioate bond
  source 1..20: mol_type = other DNA
tggaatggaatggaatggaa

SEQ ID NO: 143 - DNA, 20 bp, synthetic construct
  modified_base order(1,4,16,20): mod_base = OTHER; note = 2'-O,4'-C-ethylene-bridged nucleic acid
  modified_base order(2..3,5,17..19): mod_base = OTHER; note = 2'-O-methoxyethyl RNA
  modified_base order(1..6,8..9,11..12,14..20): mod_base = OTHER; note = phosphorothioate bond
  source 1..20: mol_type = other DNA
tggaatggaatggaatggaa

SEQ ID NO: 144 - DNA, 20 bp, synthetic construct
  modified_base order(1,4,16,20): mod_base = OTHER; note = 2'-O,4'-C-ethylene-bridged nucleic acid
  modified_base order(2..3,5,17..19): mod_base = OTHER; note = 2'-O-methoxyethyl RNA
  modified_base order(1..6,7..8,9..10,11..12,13..14,15..20): mod_base = OTHER; note = phosphorothioate bond
  source 1..20: mol_type = other DNA
tggaatggaatggaatggaa

SEQ ID NO: 145 - DNA, 20 bp, synthetic construct
  modified_base order(1,4,16,20): mod_base = OTHER; note = 2'-O,4'-C-ethylene-bridged nucleic acid
  modified_base order(2..3,5,17..19): mod_base = OTHER; note = 2'-O-methoxyethyl RNA
  modified_base order(1..9,15..20): mod_base = OTHER; note = phosphorothioate bond
  source 1..20: mol_type = other DNA
tggaatggaatggaatggaa

SEQ ID NO: 146 - DNA, 20 bp, synthetic construct
  modified_base order(1,4,16,20): mod_base = OTHER; note = 2'-O,4'-C-ethylene-bridged nucleic acid
  modified_base order(2..3,5,17..19): mod_base = OTHER; note = 2'-O-methoxyethyl RNA
  modified_base order(1..10,15..20): mod_base = OTHER; note = phosphorothioate bond
  source 1..20: mol_type = other DNA
tggaatggaatggaatggaa

SEQ ID NO: 147 - DNA, 20 bp, synthetic construct
  modified_base order(1,4,16,20): mod_base = OTHER; note = 2'-O,4'-C-ethylene-bridged nucleic acid
  modified_base order(2..3,5,17..19): mod_base = OTHER; note = 2'-O-methoxyethyl RNA
  modified_base order(1..3,5..16,18..20): mod_base = OTHER; note = phosphorothioate bond
  source 1..20: mol_type = other DNA
tggaatggaatggaatggaa

SEQ ID NO: 148 - DNA, 20 bp, synthetic construct
  modified_base order(1,4,16,20): mod_base = OTHER; note = 2'-O,4'-C-ethylene-bridged nucleic acid
  modified_base order(2..3,5,17..19): mod_base = OTHER; note = 2'-O-methoxyethyl RNA
  modified_base order(2..3,5..16,18..20): mod_base = OTHER; note = phosphorothioate bond
  source 1..20: mol_type = other DNA
tggaatggaatggaatggaa

SEQ ID NO: 149 - DNA, 20 bp, synthetic construct
  modified_base order(1,4,16,20): mod_base = OTHER; note = 2'-O,4'-C-ethylene-bridged nucleic acid
  modified_base order(2..3,5,17..19): mod_base = OTHER; note = 2'-O-methoxyethyl RNA
  modified_base order(1..3,6..17,18..20): mod_base = OTHER; note = phosphorothioate bond
  source 1..20: mol_type = other DNA
tggaatggaatggaatggaa

SEQ ID NO: 150 - DNA, 20 bp, synthetic construct
  modified_base order(1,4,16,20): mod_base = OTHER; note = 2'-O,4'-C-ethylene-bridged nucleic acid
  modified_base order(2..3,5,17..19): mod_base = OTHER; note = 2'-O-methoxyethyl RNA
  modified_base order(1..2,3..4,5..16,17..18,19..20): mod_base = OTHER; note = phosphorothioate bond
  source 1..20: mol_type = other DNA
tggaatggaatggaatggaa

SEQ ID NO: 151 - RNA, 20 nt, synthetic construct
  modified_base order(1..5,16..20): mod_base = OTHER; note = 2'-O-methyl RNA
  modified_base order(1..6,15..20): mod_base = OTHER; note = phosphorothioate bond
  source 1..20: mol_type = other RNA
ttccattccattccattcca

SEQ ID NO: 152 - DNA, 13 bp, synthetic construct
  modified_base order(1..2,11..13): mod_base = OTHER; note = locked nucleic acid
  modified_base 1..13: mod_base = OTHER; note = phosphorothioate bond
  source 1..13: mol_type = other DNA
ggaatggaatgga

SEQ ID NO: 153 - DNA, 13 bp, synthetic construct
  modified_base order(1..2,11..13): mod_base = OTHER; note = locked nucleic acid
  misc_feature 3..10: note = RNA
  modified_base 1..13: mod_base = OTHER; note = phosphorothioate bond
  modified_base 10: mod_base = OTHER; note = uracil
  source 1..13: mol_type = other DNA
ggaatggaatgga

SEQ ID NO: 154 - DNA, 13 bp, synthetic construct
  modified_base order(1,5,8,12..13): mod_base = OTHER; note = locked nucleic acid
  modified_base 1..13: mod_base = OTHER; note = phosphorothioate bond
  source 1..13: mol_type = other DNA
ggaatggaatgga

SEQ ID NO: 155 - DNA, 13 bp, synthetic construct
  modified_base order(1,5,8,12..13): mod_base = OTHER; note = locked nucleic acid
  misc_feature order(2..4,6..7,9..11): note = RNA
  modified_base 1..13: mod_base = OTHER; note = phosphorothioate bond
  modified_base 10: mod_base = OTHER; note = uracil
  source 1..13: mol_type = other DNA
ggaatggaatgga

SEQ ID NO: 156 - DNA, 13 bp, synthetic construct
  modified_base order(1..2,11..13): mod_base = OTHER; note = locked nucleic acid
  modified_base order(2,12..13): mod_base = OTHER; note = 5-methylcytosine
  modified_base 1..13: mod_base = OTHER; note = phosphorothioate bond
  source 1..13: mol_type = other DNA
tccattccattcc

SEQ ID NO: 157 - DNA, 13 bp, synthetic construct
  modified_base order(1..2,11..13): mod_base = OTHER; note = locked nucleic acid
  modified_base order(1..2,11..12): mod_base = OTHER; note = 5-methylcytosine
  modified_base 1..13: mod_base = OTHER; note = phosphorothioate bond
  source 1..13: mol_type = other DNA
ccattccattcca

SEQ ID NO: 158 - DNA, 13 bp, synthetic construct
  modified_base order(1..2,11..13): mod_base = OTHER; note = locked nucleic acid
  modified_base 1..13: mod_base = OTHER; note = phosphorothioate bond
  source 1..13: mol_type = other DNA
attccattccatt

SEQ ID NO: 159 - DNA, 13 bp, synthetic construct
  modified_base order(1..2,11..13): mod_base = OTHER; note = locked nucleic acid
  modified_base 13: mod_base = OTHER; note = 5-methylcytosine
  modified_base 1..13: mod_base = OTHER; note = phosphorothioate bond
  source 1..13: mol_type = other DNA
ttccattccattc

SEQ ID NO: 160 - DNA, 13 bp, synthetic construct
  modified_base order(1..2,11..13): mod_base = OTHER; note = locked nucleic acid
  modified_base order(1,11): mod_base = OTHER; note = 5-methylcytosine
  modified_base 1..13: mod_base = OTHER; note = phosphorothioate bond
  source 1..13: mol_type = other DNA
cattccattccat

SEQ ID NO: 161 - DNA, 13 bp, synthetic construct
  modified_base order(1..2,11..13): mod_base = OTHER; note = locked nucleic acid
  modified_base order(1..2,11): mod_base = OTHER; note = 5-methylcytosine
  modified_base 1..13: mod_base = OTHER; note = phosphorothioate bond
  source 1..13: mol_type = other DNA
ccattccattcta

SEQ ID NO: 162 - DNA, 13 bp, synthetic construct
  modified_base order(1..2,11..13): mod_base = OTHER; note = locked nucleic acid
  modified_base order(2,12): mod_base = OTHER; note = 5-methylcytosine
  modified_base 1..13: mod_base = OTHER; note = phosphorothioate bond
  source 1..13: mol_type = other DNA
tccattccattct

SEQ ID NO: 163 - DNA, 16 bp, synthetic construct
  modified_base order(1..3,14..16): mod_base = OTHER; note = locked nucleic acid
  modified_base order(1,15): mod_base = OTHER; note = 5-methylcytosine
  modified_base 1..16: mod_base = OTHER; note = phosphorothioate bond
  source 1..16: mol_type = other DNA
cattccattctattct

SEQ ID NO: 164 - DNA, 16 bp, synthetic construct
  modified_base order(1..3,14..16): mod_base = OTHER; note = locked nucleic acid
  modified_base order(1..2,16): mod_base = OTHER; note = 5-methylcytosine
  modified_base 1..16: mod_base = OTHER; note = phosphorothioate bond
  source 1..16: mol_type = other DNA
ccattccattctattc

SEQ ID NO: 165 - DNA, 16 bp, synthetic construct
  modified_base order(1..3,14..16): mod_base = OTHER; note = locked nucleic acid
  modified_base 2..3: mod_base = OTHER; note = 5-methylcytosine
  modified_base 1..16: mod_base = OTHER; note = phosphorothioate bond
  source 1..16: mol_type = other DNA
tccattccattctatt

SEQ ID NO: 166 - DNA, 16 bp, synthetic construct
  modified_base order(1..3,14..16): mod_base = OTHER; note = locked nucleic acid
  modified_base 3: mod_base = OTHER; note = 5-methylcytosine
  modified_base 1..16: mod_base = OTHER; note = phosphorothioate bond
  source 1..16: mol_type = other DNA
ttccattccattctat

SEQ ID NO: 167 - DNA, 16 bp, synthetic construct
  modified_base order(1..3,14..16): mod_base = OTHER; note = locked nucleic acid
  modified_base 14: mod_base = OTHER; note = 5-methylcytosine
  modified_base 1..16: mod_base = OTHER; note = phosphorothioate bond
  source 1..16: mol_type = other DNA
attccattccattcta

SEQ ID NO: 168 - DNA, 16 bp, synthetic construct
  modified_base order(1..3,14..16): mod_base = OTHER; note = locked nucleic acid
  modified_base order(1,15): mod_base = OTHER; note = 5-methylcytosine
  modified_base 1..16: mod_base = OTHER; note = phosphorothioate bond
  source 1..16: mol_type = other DNA
cattccattccattct

SEQ ID NO: 169 - DNA, 14 bp, synthetic construct
  modified_base order(1..3,12..14): mod_base = OTHER; note = locked nucleic acid
  modified_base order(1..3,12..13): mod_base = OTHER; note = 5-methylcytosine
  modified_base 1..14: mod_base = OTHER; note = phosphorothioate bond
  source 1..14: mol_type = other DNA
cccattccattcca

SEQ ID NO: 170 - DNA, 14 bp, synthetic construct
  modified_base order(1..3,12..14): mod_base = OTHER; note = locked nucleic acid
  modified_base order(3,14): mod_base = OTHER; note = 5-methylcytosine
  modified_base 1..14: mod_base = OTHER; note = phosphorothioate bond
  source 1..14: mol_type = other DNA
ttcccattccattc

SEQ ID NO: 171 - DNA, 14 bp, synthetic construct
  modified_base order(1..3,12..14): mod_base = OTHER; note = locked nucleic acid
  modified_base order(1,12): mod_base = OTHER; note = 5-methylcytosine
  modified_base 1..14: mod_base = OTHER; note = phosphorothioate bond
  source 1..14: mol_type = other DNA
cattcccattccat

SEQ ID NO: 172 - DNA, 14 bp, synthetic construct
  modified_base order(1..3,12..14): mod_base = OTHER; note = locked nucleic acid
  modified_base order(2..3,13..14): mod_base = OTHER; note = 5-methylcytosine
  modified_base 1..14: mod_base = OTHER; note = phosphorothioate bond
  source 1..14: mol_type = other DNA
tccattcccattcc

SEQ ID NO: 173 - DNA, 14 bp, synthetic construct
  modified_base order(1..3,12..14): mod_base = OTHER; note = locked nucleic acid
  modified_base 1..14: mod_base = OTHER; note = phosphorothioate bond
  source 1..14: mol_type = other DNA
attccattcccatt

SEQ ID NO: 174 - DNA, 14 bp, synthetic construct
  modified_base order(1..3,12..14): mod_base = OTHER; note = locked nucleic acid
  modified_base order(1..2,12..13): mod_base = OTHER; note = 5-methylcytosine
  modified_base 1..14: mod_base = OTHER; note = phosphorothioate bond
  source 1..14: mol_type = other DNA
ccattccattccca

SEQ ID NO: 175 - DNA, 14 bp, synthetic construct
  modified_base order(1..3,12..14): mod_base = OTHER; note = locked nucleic acid
  modified_base order(2..3,13..14): mod_base = OTHER; note = 5-methylcytosine
  modified_base 1..14: mod_base = OTHER; note = phosphorothioate bond
  source 1..14: mol_type = other DNA
tcccattccattcc

SEQ ID NO: 176 - DNA, 14 bp, synthetic construct
  modified_base order(1..3,12..14): mod_base = OTHER; note = locked nucleic acid
  modified_base 1..14: mod_base = OTHER; note = phosphorothioate bond
  source 1..14: mol_type = other DNA
attcccattccatt

SEQ ID NO: 177 - DNA, 14 bp, synthetic construct
  modified_base order(1..3,12..14): mod_base = OTHER; note = locked nucleic acid
  modified_base 13: mod_base = OTHER; note = 5-methylcytosine
  modified_base 1..14: mod_base = OTHER; note = phosphorothioate bond
  source 1..14: mol_type = other DNA
tagtgattgaaaca

SEQ ID NO: 178 - DNA, 14 bp, synthetic construct
  modified_base order(1..3,12..14): mod_base = OTHER; note = locked nucleic acid
  modified_base 1..14: mod_base = OTHER; note = phosphorothioate bond
  source 1..14: mol_type = other DNA
attttagtgattga

SEQ ID NO: 179 - DNA, 16 bp, synthetic construct
  modified_base order(1,3,14,16): mod_base = OTHER; note = locked nucleic acid
  modified_base order(2,15): mod_base = OTHER; note = 2'-O-methyl RNA
  modified_base 14: mod_base = OTHER; note = 5-methylcytosine
  modified_base 1..16: mod_base = OTHER; note = phosphorothioate bond
  source 1..16: mol_type = other DNA
  modified_base 2: mod_base = OTHER; note = uracil
attccattccattcca

SEQ ID NO: 180 - DNA, 16 bp, synthetic construct
  modified_base order(1,3,14,16): mod_base = OTHER; note = locked nucleic acid
  modified_base order(2,15): mod_base = OTHER; note = 2'-O-methyl RNA
  modified_base 2: mod_base = OTHER; note = uracil
  modified_base order(3,14): mod_base = OTHER; note = 5-methylcytosine
  modified_base 1..16: mod_base = OTHER; note = phosphorothioate bond
  source 1..16: mol_type = other DNA
ttccattccattccat

SEQ ID NO: 181 - DNA, 20 bp, synthetic construct
  modified_base order(1..5,16..20): mod_base = OTHER; note = 2'-O-methoxyethyl RNA
  modified_base order(3..4,18..19): mod_base = OTHER; note = 5-methylcytosine
  modified_base 1..20: mod_base = OTHER; note = phosphorothioate bond
  source 1..20: mol_type = other DNA
ttccattccattccattcca

SEQ ID NO: 182 - DNA, 20 bp, synthetic construct
  modified_base order(1..5,16..20): mod_base = OTHER; note = 2'-O-methoxyethyl RNA
  modified_base order(2..3,17..18): mod_base = OTHER; note = 5-methylcytosine
  modified_base 1..20: mod_base = OTHER; note = phosphorothioate bond
  source 1..20: mol_type = other DNA
tccattccattccattccat

SEQ ID NO: 183 - DNA, 20 bp, synthetic construct
  modified_base order(1..5,16..20): mod_base = OTHER; note = 2'-O-methoxyethyl RNA
  modified_base order(1..2,16..17): mod_base = OTHER; note = 5-methylcytosine
  modified_base 1..20: mod_base = OTHER; note = phosphorothioate bond
  source 1..20: mol_type = other DNA
ccattccattccattccatt

SEQ ID NO: 184 - DNA, 20 bp, synthetic construct
  modified_base order(1..5,16..20): mod_base = OTHER; note = 2'-O-methoxyethyl RNA
  modified_base order(1,5,16,20): mod_base = OTHER; note = 5-methylcytosine
  modified_base 1..20: mod_base = OTHER; note = phosphorothioate bond
  source 1..20: mol_type = other DNA
cattccattccattccattc

SEQ ID NO: 185 - DNA, 20 bp, synthetic construct
  modified_base order(1..5,16..20): mod_base = OTHER; note = 2'-O-methoxyethyl RNA
  modified_base order(4..5,19..20): mod_base = OTHER; note = 5-methylcytosine
  modified_base 1..20: mod_base = OTHER; note = phosphorothioate bond
  source 1..20: mol_type = other DNA
attccattccattccattcc

SEQ ID NO: 186 - DNA, 13 bp, synthetic construct
  modified_base order(1..2,11..13): mod_base = OTHER; note = locked nucleic acid
  modified_base order(2,12..13): mod_base = OTHER; note = 5-methylcytosine
  modified_base 1..13: mod_base = OTHER; note = phosphorothioate bond
  source 1..13: mol_type = other DNA
tccattccattcc

SEQ ID NO: 187 - DNA, 13 bp, synthetic construct
  modified_base order(1..2,11..13): mod_base = OTHER; note = locked nucleic acid
  modified_base 3..10: mod_base = OTHER; note = 2'-O-methyl RNA
  modified_base 10: mod_base = OTHER; note = uracil
  modified_base order(2,12..13): mod_base = OTHER; note = 5-methylcytosine
  modified_base 1..13: mod_base = OTHER; note = phosphorothioate bond
  source 1..13: mol_type = other DNA
tccattccattcc

SEQ ID NO: 188 - DNA, 13 bp, synthetic construct
  modified_base order(1,5,8,12..13): mod_base = OTHER; note = locked nucleic acid
  modified_base order(8,12..13): mod_base = OTHER; note = 5-methylcytosine
  modified_base 1..13: mod_base = OTHER; note = phosphorothioate bond
  source 1..13: mol_type = other DNA
tccattccattcc

SEQ ID NO: 189 - DNA, 13 bp, synthetic construct
  modified_base order(1,5,8,12..13): mod_base = OTHER; note = locked nucleic acid
  modified_base order(2..4,6..7,9..11): mod_base = OTHER; note = 2'-O-methyl RNA
  modified_base order(8,12..13): mod_base = OTHER; note = 5-methylcytosine
  modified_base 1..13: mod_base = OTHER; note = phosphorothioate bond
  source 1..13: mol_type = other DNA
tccattccattcc

SEQ ID NO: 190 - DNA, 24 bp, Homo sapiens
  source 1..24: mol_type = genomic DNA
caacaaaacaagactctgtttcaa

SEQ ID NO: 191 - skipped

SEQ ID NO: 192 - DNA, 29 bp, Homo sapiens
  source 1..29: mol_type = genomic DNA
taataaaataaaaagaaacctctaaattg

SEQ ID NO: 193 - DNA, 24 bp, Homo sapiens
  source 1..24: mol_type = genomic DNA
ttgaaacagagtcttgttttgttg

SEQ ID NO: 194 - skipped

SEQ ID NO: 195 - DNA, 29 bp, Homo sapiens
  source 1..29: mol_type = genomic DNA
caatttagaggtttctttttattttatta

SEQ ID NO: 196 - skipped

SEQ ID NO: 197 - DNA, 22 bp, Homo sapiens
  source 1..22: mol_type = genomic DNA
ttctattcccattccattccca

SEQ ID NO: 198 - skipped

SEQ ID NO: 199 - DNA, 22 bp, Homo sapiens
  source 1..22: mol_type = genomic DNA
tgggaatggaatgggaatagaa

SEQ ID NO: 200 - DNA, 13 bp, synthetic construct
  modified_base order(1..3,11..13): mod_base = OTHER; note = 2'-O-methoxyethyl RNA
  modified_base order(1..2,11..12): mod_base = OTHER; note = 5-methylcytosine
  source 1..13: mol_type = other DNA
ccattccattcca

SEQ ID NO: 201 - DNA, 13 bp, synthetic construct
  modified_base 1..13: mod_base = OTHER; note = 2'-O-methoxyethyl RNA
  modified_base order(1..2,6..7,11..12): mod_base = OTHER; note = 5-methylcytosine
  source 1..13: mol_type = other DNA
ccattccattcca

SEQ ID NO: 202 - DNA, 13 bp, synthetic construct
  modified_base order(1..3,6..7,11..13): mod_base = OTHER; note = 2'-O-methoxyethyl RNA
  modified_base order(1..2,6..7,11..12): mod_base = OTHER; note = 5-methylcytosine
  source 1..13: mol_type = other DNA
ccattccattcca

SEQ ID NO: 203 - DNA, 13 bp, synthetic construct
  modified_base order(1..3,6..8,11..13): mod_base = OTHER; note = 2'-O-methoxyethyl RNA
  modified_base order(1..2,6..7,11..12): mod_base = OTHER; note = 5-methylcytosine
  source 1..13: mol_type = other DNA
ccattccattcca

SEQ ID NO: 204 - DNA, 14 bp, synthetic construct
  modified_base order(1..3,12..14): mod_base = OTHER; note = locked nucleic acid
  modified_base 1..14: mod_base = OTHER; note = phosphorothioate bond
  source 1..14: mol_type = other DNA
ggaatggaatggaa

SEQ ID NO: 205 - DNA, 14 bp, synthetic construct
  modified_base order(1..3,12..14): mod_base = OTHER; note = locked nucleic acid
  modified_base 1..14: mod_base = OTHER; note = phosphorothioate bond
  source 1..14: mol_type = other DNA
tggaatggaatgga

SEQ ID NO: 206 - DNA, 14 bp, synthetic construct
  modified_base order(1..3,12..14): mod_base = OTHER; note = locked nucleic acid
  modified_base 1..14: mod_base = OTHER; note = phosphorothioate bond
  source 1..14: mol_type = other DNA
atggaatggaatgg

SEQ ID NO: 207 - DNA, 14 bp, synthetic construct
  modified_base order(1..3,12..14): mod_base = OTHER; note = locked nucleic acid
  source 1..14: mol_type = other DNA
  modified_base order(3,13..14): mod_base = OTHER; note = 5-methylcytosine
ttccattccattcc

SEQ ID NO: 208 - DNA, 14 bp, synthetic construct
  modified_base order(1..3,12..14): mod_base = OTHER; note = locked nucleic acid
  source 1..14: mol_type = other DNA
  modified_base order(2..3,12..13): mod_base = OTHER; note = 5-methylcytosine
tccattccattcca

SEQ ID NO: 209 - DNA, 14 bp, synthetic construct
  modified_base order(1..3,12..14): mod_base = OTHER; note = locked nucleic acid
  source 1..14: mol_type = other DNA
  modified_base order(1..2,12): mod_base = OTHER; note = 5-methylcytosine
ccattccattccat